(19) United States
(12) Patent Application Publication
(10) Pub. No.: US 2008/0187969 A1
(43) Pub. Date: Aug. 7, 2008

(54) Nucleic acid amplification using non-random primers

(75) Inventors: John Castle, Seattle, WA (US); Christopher K. Raymond, Seattle, WA (US); Christopher Armour, Kirkland, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Rosetta Inpharmatics LLC, Seattle, WA (US)

(21) Appl. No.: 11/589,322

(22) Filed: Oct. 27, 2006

Related U.S. Application Data
(60) Provisional application No. 60/731,529, filed on Oct. 27, 2005.
(60) Provisional application No. 60/790,458, filed on Apr. 7, 2006.

(51) Int. Cl. C12P 19/34; C07H 21/00

(52) U.S. Cl. 435/91.2; 435/91.1; 536/23.1

(57) ABSTRACT

The present invention provides methods for selectively amplifying a target population of nucleic acid molecules (e.g., all mRNA molecules expressed in a cell type except for the most highly expressed mRNA species). The present invention also provides populations of oligonucleotides including the nucleic acid sequences set forth in SEQ ID NOS:1-933. These oligonucleotides can be used, for example, to prime the synthesis of cDNA molecules complementary to mRNA molecules isolated from mammalian blood without priming the synthesis of cDNA molecules complementary to globin mRNA, or ribosomal RNA molecules.

### CROSS-REFERENCE TO RELATED APPLICATIONS

This application claims the benefit of U.S. Provisional Application No. 60/790,458, filed Apr. 7, 2006, and also claims the benefit of U.S. Provisional Application No. 60/731,529, filed Oct. 27, 2005.

### FIELD OF THE INVENTION

The present invention relates to oligonucleotides useful for priming the amplification of nucleic acid molecules.

### BACKGROUND OF THE INVENTION

Gene expression analysis often involves amplification of starting nucleic acid molecules. Amplification of nucleic acid molecules may be accomplished by reverse transcription (RT), in vitro transcription (IVT) or the polymerase chain reaction (PCR), either individually or in combination. The starting nucleic acid molecules may be mRNA molecules, which are amplified by first synthesizing complementary cDNA molecules, then synthesizing second cDNA molecules that are complementary, to the first cDNA molecules, thereby producing double stranded cDNA molecules. The synthesis of first strand cDNA is typically accomplished using a reverse transcriptase and the synthesis of second strand cDNA is typically accomplished using a DNA polymerase. The double stranded cDNA molecules may be used to make complementary RNA molecules using an RNA polymerase, resulting in amplification of the original starting mRNA molecules. The RNA polymerase requires a promoter sequence to direct initiation of RNA synthesis. Complementary RNA molecules may, for example, be used as a template to make additional complementary DNA molecules.

Amplification of nucleic acid molecules requires the use of oligonucleotide primers that specifically hybridize to one or more target nucleic acid molecules in the starting material. Each oligonucleotide primer may include a promoter sequence that is located 5′ to the hybridizing portion of the oligonucleotide that hybridizes to the target nucleic acid molecule(s). If the hybridizing portion of an oligonucleotide is too short, then the oligonucleotide does not stably hybridize to a target nucleic acid molecule and priming and subsequent amplification does not occur. Also, if the hybridizing portion of an oligonucleotide is too short, then the oligonucleotide does not specifically hybridize to one or a small number of target nucleic acid molecules, but non-specifically hybridizes to numerous target nucleic acid molecules.

Amplification of a complex mixture of different target nucleic acid molecules (e.g., mRNA molecules) typically requires the use of a population of numerous oligonucleotides having different nucleic acid sequences. The cost of the oligonucleotides increases with the length of the oligonucleotides. In order to control costs, it is preferable to make oligonucleotide primers that are no longer than the minimum length required to ensure specific hybridization of an oligonucleotide to a target sequence.

It is often undesirable to amplify highly expressed RNAs (e.g., ribosomal RNAs). For example, in gene expression experiments that analyze expression of genes in blood cells, amplification of numerous copies of abundant globin mRNAs, or ribosomal RNAs, may obscure subtle changes in the levels of rare mRNAs. Thus, there is a need for populations of oligonucleotide primers that selectively amplify desired nucleic acid molecules within a population of nucleic acid molecules (e.g., oligonucleotide primers that selectively amplify all mRNAs that are expressed in a cell except for the most highly expressed mRNAs). In order to reduce the cost of synthesizing the population of oligonucleotides, the hybridizing portion of each oligonucleotide should be no longer than necessary to ensure specific hybridization to a desired target sequence under defined conditions.

### SUMMARY OF THE INVENTION

In one aspect, the present invention provides methods for selectively amplifying a target population of nucleic acid molecules (e.g., all mRNA molecules expressed in a cell type except for the most highly expressed mRNA species). The methods of this aspect of the invention each include the step of using a population of oligonucleotides to prime the amplification of a target population of nucleic acid molecules within a larger population of nucleic acid molecules. The population of oligonucleotides is selected based on its ability to hybridize under defined conditions to a first subpopulation of a target nucleic acid population, but not to hybridize under the defined conditions to a second subpopulation of the target nucleic acid population.

In another aspect, the present invention provides populations of oligonucleotides including the nucleic acid sequences set forth in SEQ ID NOS:1-933. These oligonucleotides can be used, for example, to prime the synthesis of cDNA molecules complementary to mRNA molecules isolated from mammalian blood without priming the synthesis of cDNA molecules complementary to globin mRNA or ribosomal RNA molecules. In some embodiments, each oligonucleotide in the population of oligonucleotides further comprises a defined sequence portion located 5′ to the hybridizing portion. In one embodiment, the defined sequence portion comprises a transcriptional promoter, which may be used as a primer binding site in PCR amplification, or for in vitro transcription. In another embodiment, the defined sequence portion comprises a primer binding site that is not a transcriptional promoter. For example, in some embodiments the present invention provides populations of oligonucleotides wherein a transcriptional promoter, such as the T7 promoter (SEQ ID NO:934), is located 5′ to a member of the population of oligonucleotides having the sequences set forth in SEQ ID NOS:1-933. Thus, in some embodiments, the present invention provides populations of oligonucleotides wherein each oligonucleotide consists of the T7 promoter (SEQ ID NO:934) located 5′ to a different member of the population of oligonucleotides having the sequences set forth in SEQ ID NOS:1-933. In further embodiments, the present invention provides populations of oligonucleotides wherein the defined sequence portion comprises at least one primer binding site which is useful for priming a PCR synthesis reaction, and which does not include an RNA polymerase promoter sequence. A representative example of a defined sequence portion for use in such embodiments is provided as 5′ CCGAACTACCCACTTGCATT 3′ (SEQ ID NO:956), which is preferably located 5′ to a member of the population of oligonucleotides having the sequences set forth in SEQ ID NOS:1-933.

In another aspect, the present invention provides a reagent for selectively amplifying a target population of nucleic acid molecules (e.g., all mRNA molecules expressed in a cell type except for the most highly expressed mRNA species). The reagent of this aspect of the invention comprises a population of oligonucleotides to prime the amplification of a target population of nucleic acid molecules, wherein each oligonucleotide comprises a hybridizing portion that consists of 6, 7, or 8 nucleotides. In some embodiments, the present invention provides a reagent comprising a population of oligonucleotides wherein the hybridizing portion is a member of the population of oligonucleotides having the sequences set forth in SEQ ID NOS:1-933. Thus, in some embodiments, the present invention provides a reagent comprising a population of oligonucleotides that includes at least 10% (such as at least 20%, 30%, 40%, 50%, 60%, 70%, 80%, 85%, 90%, 95%, or 9.9%) of the sequences set forth in SEQ ID NOS:1-933. In another embodiment, the present invention provides a reagent comprising populations of oligonucleotides wherein a defined sequence portion, such as a transcriptional promoter, (e.g., the T7 promoter (SEQ ID NO:934)) or a primer binding site (e.g., SEQ ID NO:956) is located 5′ to a member of the population of oligonucleotides having the sequences set forth in SEQ ID NOS:1-933. Thus, in some embodiments, the present invention provides a reagent comprising populations of oligonucleotides wherein each oligonucleotide consists of the T7 promoter (SEQ ID NO:934) located 5′ to a different member of the population of oligonucleotides having the sequences set forth in SEQ ID NOS:1-933.

In a further aspect, the present invention provides a kit for selectively amplifying a target population of nucleic acid molecules (e.g., all mRNA molecules expressed in a cell type except for the most highly expressed mRNA species). The kit of this aspect of the invention includes a reagent comprising a population of oligonucleotides to prime the amplification of a target population of nucleic acid molecules, wherein each oligonucleotide comprises a hybridizing portion that consists of 6, 7, or 8 nucleotides. In some embodiments, the present invention provides a kit that includes a reagent comprising a population of oligonucleotides wherein the hybridizing portion is a member of the population of oligonucleotides having the sequences set forth in SEQ ID NOS:1-933. Thus, in some embodiments, the present invention provides a kit including a reagent that comprises a population of oligonucleotides that includes at least 10% (such as at least 20%, 30%, 40%, 50%, 60%, 70%, 80%, 85%, 90%, 95%, or 99%) of the sequences set forth in SEQ ID NOS:1-933. In another embodiment, the present invention provides a kit that includes a reagent comprising populations of oligonucleotides wherein a defined sequence portion, such as the transcriptional promoter, (e.g., the T7 promoter (SEQ ID NO:934)), is located 5′ to a member of the population of oligonucleotides having the sequences set forth in SEQ ID NOS:1-933. Thus, in some embodiments, the present invention provides a kit including a reagent that comprises populations of oligonucleotides wherein each oligonucleotide consists of the T7 promoter (SEQ ID NO:934) located 5′ to a different member of the population of oligonucleotides having the sequences set forth in SEQ ID NOS:1-933.

In another aspect, the present invention provides methods of selectively amplifying a target population of nucleic acid molecules to generate amplified RNA molecules. The method comprises: (a) providing a population of oligonucleotides, wherein each oligonucleotide comprises a hybridizing portion and transcriptional promoter portion located 5′ to the hybridizing portion, wherein the hybridizing portion is a member of the population of oligonucleotides comprising SEQ ID NOS:1-933, (b) annealing the population of oligonucleotides to a sample comprising mRNA isolated from a mammalian subject, (c) synthesizing cDNA from the mRNA using a reverse transcriptase enzyme, (d) synthesizing double stranded cDNA using a DNA polymerase; and (e) transcribing the double-stranded cDNA into RNA using an RNA polymerase that binds to the transcriptional promoter portion of each oligonucleotide to generate amplified RNA.

In another aspect, the present invention provides methods of selectively amplifying a target population of nucleic acid molecules to generate amplified DNA molecules. The method comprises: (a) providing a first population of oligonucleotides, wherein each oligonucleotide comprises a hybridizing portion and a first PCR primer binding site located 5′ to the hybridizing portion, wherein the hybridizing portion is a member of the population of oligonucleotides comprising SEQ ID NOS:1-933, (b) annealing the population of oligonucleotides to a sample comprising mRNA isolated from a mammalian subject, (c) synthesizing cDNA from the mRNA using a reverse transcriptase enzyme, (d) synthesizing double stranded cDNA using a DNA polymerase and a second population of oligonucleotides, wherein each oligonucleotide comprises a random hybridizing portion and a second PCR binding site located 5′ to the hybridizing portion, and (e) PCR amplifying the double stranded cDNA using thermostable DNA polymerase, a first PCR primer that binds to the first PCR primer binding site and a second PCR primer that binds to the second PCR primer binding site to generate amplified double stranded DNA.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A shows the number of exact matches for random 6-mers (N6) oligonucleotides on nucleotide sequences in the human RefSeq transcript database as described in EXAMPLE 2;

FIG. 1B shows the number of exact matches for Not-So-Random (NSR) 6-mer oligonucleotides on nucleotide sequences in the human RefSeq transcript database as described in EXAMPLE 2;

FIG. 1C shows a representative embodiment of the methods of the invention for synthesizing a preparation of amplified RNA molecules using a mixture of NSR 6-mer oligonucleotides as described in EXAMPLE 4;

FIG. 1D shows a representative embodiment of the methods of the invention for synthesizing a preparation of amplified DNA molecules using a mixture of NSR 6-mer oligonucleotides as described in EXAMPLE 12;

FIG. 2 is a histogram plot showing the number of NSR 6-mer binding sites per 100 nucleotides present in the human RefSeq transcript database as described in EXAMPLE 2;

FIG. 3 is a histogram plot showing the yield of amplified RNA from T7-NSR6-mer primed cDNA as a function of input RNA template amount as described in EXAMPLE 6;

FIG. 4 is a histogram plot showing the yield of amplified RNA from T7-NSR6-mer and T7-N8 primed cDNA for reporter and background genes as a function of primer and dNTP concentrations as described in EXAMPLE 7;

FIG. 5 is a histogram plot showing the yield of amplified RNA from T7-NSR and T7-N7 primed cDNA as a function of the amount of input RNA template as described in EXAMPLE 9;

FIG. 6 is a histogram plot on a linear scale showing the relative composition of T7-NSR and T7-N7 primed cDNA (normalized to N8) following amplification by in vitro transcription as described in EXAMPLE 9;

FIG. 7 is a histogram plot on a logarithmic scale showing the relative composition of T7-NSR and T7-N7 primed cDNA (normalized to N8) following amplification by in vitro transcription as described in EXAMPLE 9;

FIG. 8 is a histogram plot of relative abundance of in vitro transcription products from T7-NSR-primed cDNA as a function of RNA template amount as described in EXAMPLE 9;

FIG. 9 is a histogram plot of relative abundance of in vitro transcription products from T7-N-7-primed cDNA as a function of RNA template amount as described in EXAMPLE 9;

FIG. 10 graphically illustrates the correlation in expression values from a panel of 34 reporter genes as measured by quantitative PCR of T7-NSR-primed cDNA (x-axis) versus amplified DNA (aDNA) (y-axis) generated from T7-NSR-primed cDNA as described in EXAMPLE 12; and

FIG. 11 is a histogram plot of the gene specific activities of a panel of 10 reporter genes as measured by quantitative PCR of amplified DNA generated using a range of primer concentrations.

### DETAILED DESCRIPTION OF THE PREFERRED EMBODIMENT

Unless specifically defined herein, all terms used herein have the same meaning as they would to one skilled in the art of the present invention. Practitioners are particularly directed to Sambrook et al., Molecular Cloning: A Laboratory Manual, 2d ed., Cold Spring Harbor Press, Plainsview, N.Y.; and Ausubel et al., Current Protocols in Molecular Biology (Supplement 47), John Wiley & Sons, New York, 1999, for definitions and terms of the art.

In one aspect, the present invention provides methods for selectively amplifying a target population of nucleic acid molecules. The methods of this aspect of the invention each include the step of using a population of oligonucleotides to prime the amplification (e.g., by reverse transcription, in vitro transcription, or polymerase chain reaction (PCR), or a combination thereof) of a target population of nucleic acid molecules within a larger population of nucleic acid molecules, wherein (a) each oligonucleotide comprises a hybridizing portion that consists of 6 nucleotides, or 7 nucleotides, or 8 nucleotides; and (b) the population of oligonucleotides is selected to hybridize under defined conditions to a first subpopulation of a target nucleic acid population, but not hybridize under the defined conditions to a second subpopulation of the target nucleic acid population. The population of oligonucleotides may also include a defined sequence portion located 5′ to the hybridizing portion. In one embodiment, the defined sequence portion comprises a transcriptional promoter, which can also be used as a primer binding site. Therefore, in certain embodiments of this aspect of the invention, each oligonucleotide of the population of oligonucleotides comprises a hybridizing portion that consists of 6 nucleotides, or 7 nucleotides, or 8 nucleotides; and a transcriptional promoter portion located 5′ to the hybridizing portion. In another embodiment, the defined sequence portion includes a first primer binding site for use in a PCR amplification reaction, and which may optionally include a transcriptional promoter. By way of example, the populations of oligonucleotides provided by the present invention are useful in the practice of the methods of this aspect of the invention.

For example, in one embodiment of the present invention, a population of oligonucleotides (SEQ ID NOS:1-933), that each have a length of 6 nucleotides, was identified that can be used as primers to prime the amplification of all, or substantially all, mRNA molecules from mammalian blood cells, but that do not prime the amplification of globin mRNA or ribosomal RNAs from mammalian blood cells. The identified population of oligonucleotides (SEQ ID NOS:1-933) is referred to as Not-So-Random (NSR) primers. Thus, this population of oligonucleotides (SEQ ID NOS:1-933) can be used to prime the synthesis of a population of nucleic acid molecules (e.g., cDNAs) that are representative of a starting population of mRNA molecules isolated from mammalian blood cells, but that does not include a large number of cDNA molecules that correspond to globin mRNAs or to ribosomal RNAs. The present invention also provides populations of oligonucleotides wherein a defined sequence, such as the T7 promoter (SEQ ID NO:934), is located 5′ to a member of the population of oligonucleotides having the sequences set forth in SEQ ID NOS:1-933. Thus, each oligonucleotide may include a hybridizing portion (selected from SEQ ID NOS:1-933) that hybridizes to target nucleic acid molecules (e.g., mRNAs), and a promoter sequence located 5′ to the hybridizing portion. The promoter sequence may be incorporated into DNA molecules amplified using the oligonucleotides (that include the T7 promoter) as primers, and can thereafter promote transcription from the DNA molecules. Alternatively, a defined sequence portion such as a transcriptional promoter may be covalently attached to the cDNA molecule, for example, by DNA ligase enzyme.

The first subpopulation of a target nucleic acid population can include, for example, all mRNAs expressed in a cell or tissue except for a selected group of mRNAs, such as, for example, the most abundantly expressed mRNAs. An abundantly expressed mRNA typically constitutes at least 0.1% of all the mRNA expressed in the cell or tissue (and may constitute, for example, more than 50%, or more than 60%, or more than 70% of all the mRNA expressed in the cell or tissue). An example of an abundantly expressed mRNA is globin mRNA in certain blood cells. Useful transcription promoter sequences include the T7 promoter (5′ AATTAATACGACTCACTATAGGGAGA3′) (SEQ ID NO:934)), the SP6 promoter (5′ ATTTAGGTGACACTATAGAAGNG3′ (SEQ ID NO:935)), and the T3 promoter (5′ AATTAACCCTCACTAAAGGGAGA3′ (SEQ ID NO:936)).

The methods of the invention are useful for transcriptome profiling of total RNA in a biological sample, such as whole blood, in which it is desirable to reduce the presence of a group of mRNAs (that do not hybridize to the NSR primers) from an amplified sample, such as, for example, highly expressed RNAs (e.g. globin mRNA or ribosomal RNAs). In some embodiments, the methods of the invention may be used to reduce the amount of a group of nucleic acid molecules that do not hybridize to the NSR primers in amplified nucleic acid derived from an mRNA sample by at least 2 fold up to 1000 fold, such as at least 10 fold, 50 fold, 100 fold, 500 fold, or greater, in comparison to the amount of amplified nucleic acid molecules that do hybridize to the NSR primers.

Populations of oligonucleotides used to practice the method of this aspect of the invention are selected from within a larger population of oligonucleotides, wherein (a) the subpopulation of oligonucleotides is selected based on its ability to hybridize under defined conditions to a first subpopulation of a target nucleic acid population, but not hybridize under the defined conditions to a second subpopulation of the target nucleic acid population; and (b) the population of oligonucleotides comprises all possible oligonucleotides having a length of 6 nucleotides, 7 nucleotides, or 8 nucleotides.

Composition of population of oligonucleotides. The population of oligonucleotides includes all possible oligonucleotides having a length of 6 nucleotides, or 7 nucleotides, or 8 nucleotides. The population of oligonucleotides may include only all possible oligonucleotides having a length of 6 nucleotides, or all possible oligonucleotides having a length of 7 nucleotides, or all possible oligonucleotides having a length of 8 nucleotides. Optionally the population of oligonucleotides may include other oligonucleotides in addition to all possible oligonucleotides having a length of 6 nucleotides, or all possible oligonucleotides having a length of 7 nucleotides, or all possible oligonucleotides having a length of 8 nucleotides. Typically, each member of the population of oligonucleotides is no more than 30 nucleotides long.

Sequences of population of oligonucleotides. There are 4,096 possible oligonucleotides having a length of 6 nucleotides; 16,384 possible oligonucleotides having a length of 7 nucleotides; and 65,536 possible oligonucleotides having a length of 8 nucleotides. The sequences of the oligonucleotides that constitute the population of oligonucleotides can readily be generated by a computer program, such as Microsoft Word.

Selection of subpopulation of oligonucleotides. The subpopulation of oligonucleotides is selected from the population of oligonucleotides based on the ability of the members of the subpopulation of oligonucleotides to hybridize, under defined conditions, to a first subpopulation of a target nucleic acid population, but not hybridize under the same defined conditions to a substantial number of members of a second subpopulation of the target nucleic acid population. A target nucleic acid population is a population of nucleic acid molecules (e.g., mRNA or DNA molecules) that includes nucleic acid molecules that are to be amplified (e.g., using reverse transcription, in vitro transcription, the polymerase chain reaction, or a combination thereof) to produce amplified RNA, single stranded DNA, or double stranded DNA, and also includes nucleic acid molecules that are not to be amplified. The subpopulation of oligonucleotides is made up of oligonucleotides that each hybridize, under defined conditions, to sequences distributed throughout the population of the nucleic acid molecules that are to be amplified, but that do not hybridize, under the same defined conditions, to most (or any) of the nucleic acid molecules that are not to be amplified. The subpopulation of oligonucleotides hybridizes, under defined conditions, to target nucleic acid sequences other than those that have been intentionally avoided.

For example, the population of all mRNA molecules expressed in mammalian blood cells (e.g., human blood cells) can be a target population of nucleic acid molecules. This target population contains many mRNA molecules that encode globin proteins. This target population also contains many ribosomal RNA molecules (e.g., 5S, 18S, and 28S ribosomal RNAs). It is typically undesirable to amplify the globin mRNAs or the ribosomal RNAs. For example, in gene expression experiments that analyze expression of genes in blood cells, amplification of numerous copies of abundant globin mRNAs, or ribosomal RNAs, may obscure subtle changes in the levels of less abundant mRNAs. Consequently, in the practice of the present invention, a subpopulation of oligonucleotides is selected that does not hybridize, under defined conditions, to most (or any) globin mRNAs or to most (or any) ribosomal RNAs, but that does hybridize, under the same defined conditions, to most (preferably all) of the other mRNA molecules expressed in the blood cells. Examples of blood cells include leukocytes (e.g., neutrophils, eosinophils, basophils, lymphocytes, and monocytes), erythrocytes, and platelets.

In order to select a subpopulation of oligonucleotides that hybridizes under defined conditions to a first subpopulation of a target nucleic acid population, but does not hybridize under the defined conditions to a second subpopulation of the target nucleic acid population, it is necessary to know the complete, or substantially complete, nucleic acid sequences of the member(s) of the second subpopulation of the target nucleic acid population. Thus, for example, if the second subpopulation includes globin mRNAs and ribosomal RNAs, it is necessary to know the nucleic acid sequences of the globin mRNAs (or a representative globin mRNA) and the 5S, 18S, and 28S ribosomal RNAs (or a representative member of each of the foregoing classes of ribosomal RNA).

A suitable software program is then used to compare the sequences of all of the oligonucleotides in the population of oligonucleotides (e.g., the population of all possible 6 nucleic acid oligonucleotides) to the sequences of the globin mRNA(s) and ribosomal RNAs to determine which of the oligonucleotides will hybridize to any portion of the globin mRNA(s) and ribosomal RNAs under defined hybridization conditions. Only the oligonucleotides that do not hybridize to any portion of the globin mRNA(s) and ribosomal RNAs, under defined hybridization conditions, are selected. Perl script may easily be written that permits comparison of nucleic acid sequences, and identification of sequences that hybridize to each other under defined hybridization conditions.

Thus, for example, as described more fully in Example 1, the subpopulation of all possible 6 nucleic acid oligonucleotides that were not exactly complementary to any portion of any ribosomal RNA sequence or that were not exactly complementary to any portion of a globin mRNA sequence were identified. In general, the subpopulation of oligonucleotides (that hybridizes under defined conditions to a first subpopulation of a target nucleic acid population, but does not hybridize under the defined conditions to a second subpopulation of the target nucleic acid population) must contain enough different oligonucleotide sequences to hybridize to all, or substantially all, nucleic acid molecules in the target nucleic acid population. Example 2 herein shows that the population of oligonucleotides having the nucleic acid sequences set forth in SEQ ID NOS:1-933 hybridizes to all, or substantially all, nucleic acid sequences within a population of gene transcripts stored in the publicly accessible database called RefSeq.

Additional defined nucleic acid sequence portions. The selected subpopulation of oligonucleotides (e.g., SEQ ID NOS:1-933) can be used to prime the amplification of a target population of nucleic acid molecules. Alternatively, a population of oligonucleotides can be used as primers wherein each oligonucleotide includes the sequence of one member of the selected subpopulation of oligonucleotides, and also includes an additional defined nucleic acid sequence. The additional defined nucleic acid sequence is typically located 5′ to the sequence of the member of the selected subpopulation of oligonucleotides. Typically, the population of oligonucleotides includes the sequences of all members of the selected subpopulation of oligonucleotides (e.g., the population of oligonucleotides can include all of the sequences set forth in SEQ ID NOS:1-933).

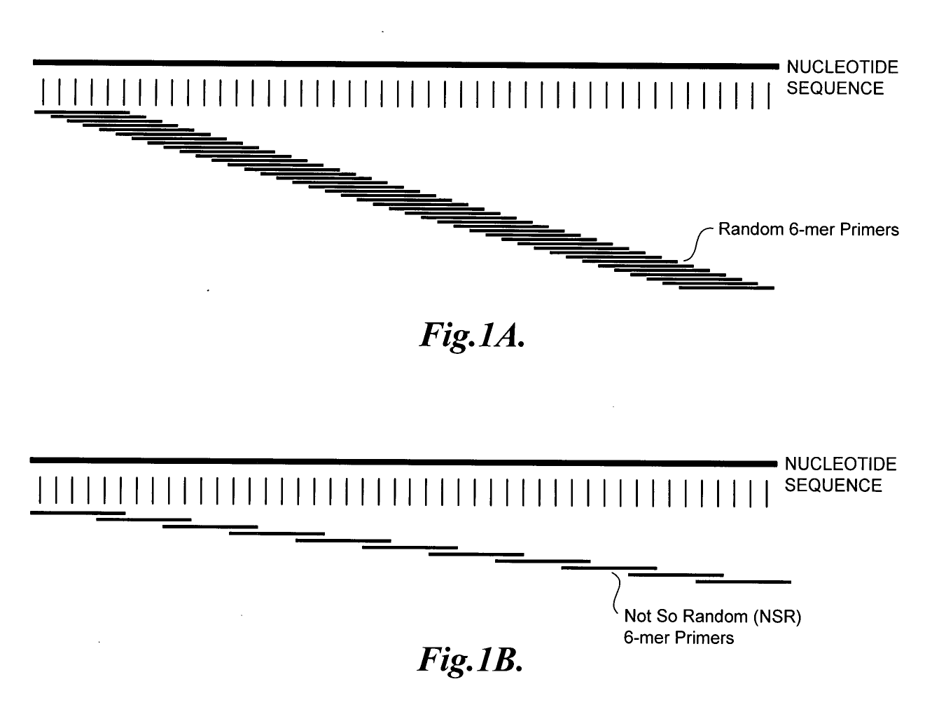

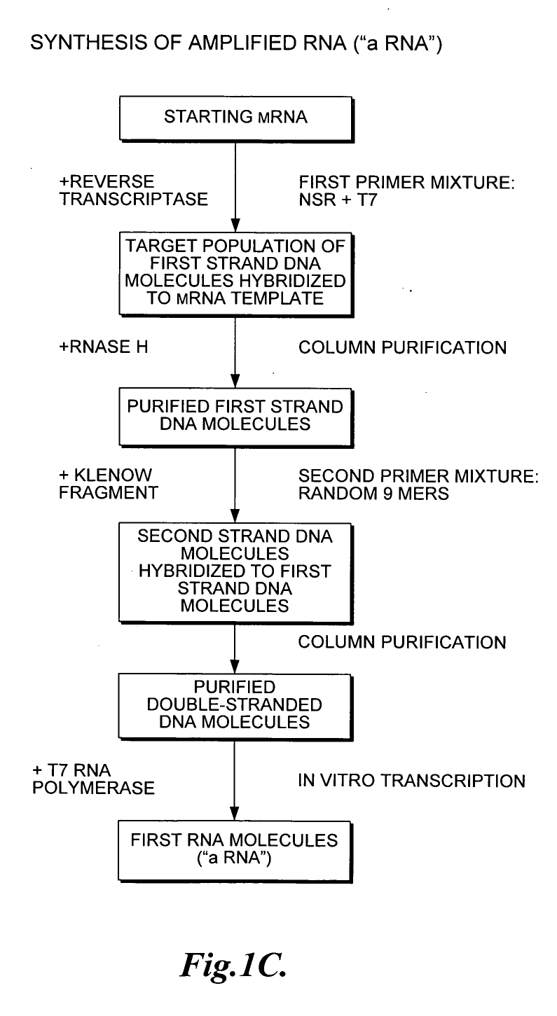

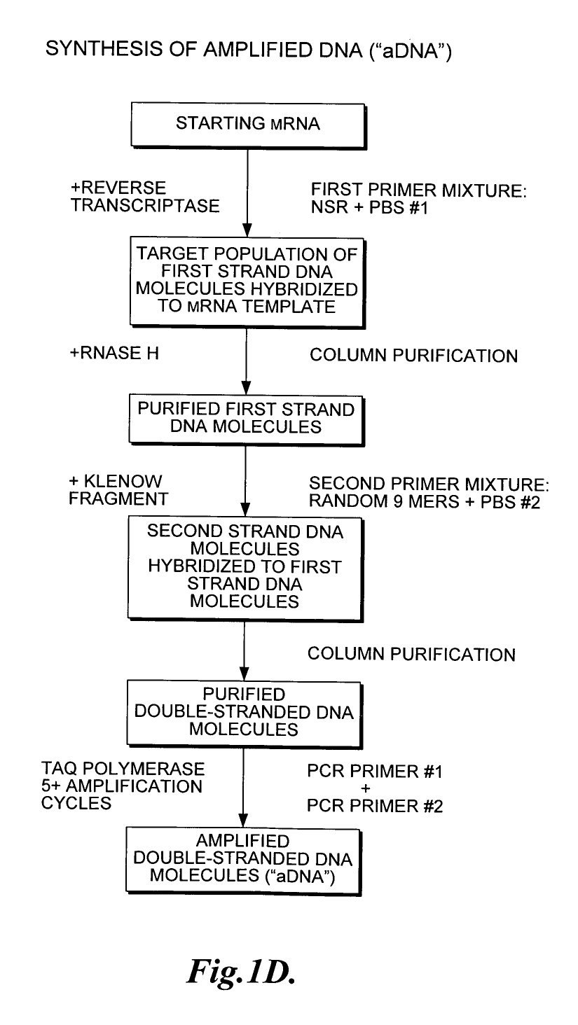

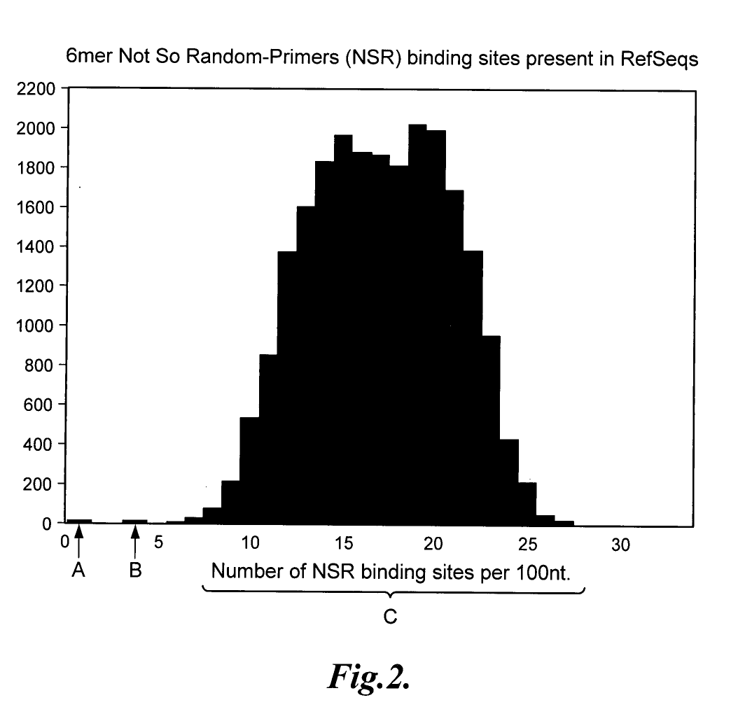

The additional defined nucleic acid sequence is selected so that it does not affect the hybridization specificity of the oligonucleotide to a complementary target sequence. For example, as described in FIG. 1C, each, oligonucleotide can include a transcriptional promoter sequence located 5′ to the sequence of the member of the selected subpopulation of oligonucleotides. The promoter sequence may be incorporated into the amplified nucleic acid molecules which can, therefore, be used as templates for the synthesis of RNA. Any RNA polymerase promoter sequence can be included in the defined sequence portion of the population of oligonucleotides. Representative examples include the T7 promoter (SEQ ID NO:934), the SP6 promoter (SEQ ID NO:935), and the T3 promoter (SEQ ID NO:936).

In another example, as described in FIG. 1D, each oligonucleotide can include a defined sequence comprising a primer binding site located 5′ to the sequence of the member of the selected subpopulation of oligonucleotides. The primer binding site is incorporated into the amplified nucleic acids which can then be used as a PCR primer binding site for the generation of double-stranded amplified DNA products from the cDNA. The primer binding site may be a portion of a transcriptional promoter sequence, as shown for example in TABLE 35. Alternatively, the primer binding site may not include a portion of a transcriptional promoter sequence, (e.g., SEQ ID NO:956, as described in Example 11).

For example, one embodiment of the present invention provides a population of oligonucleotides wherein each oligonucleotide of the population includes: (a) a sequence of a 6 nucleic acid oligonucleotide that is a member of a subpopulation of oligonucleotides (SEQ ID NOS:1-933), wherein the subpopulation of oligonucleotides hybridizes to all, or substantially all, mRNAs expressed in mammalian blood cells, but does not hybridize to globin mRNAs or to ribosomal RNAs; and (b) a T7 transcriptional promoter sequence (SEQ ID NO:934) located 5′ to the sequence of the 6 nucleic acid oligonucleotide. In one embodiment, the population of oligonucleotides includes all of the 6 nucleotide sequences set forth in SEQ ID NOS:1-933. In another embodiment, the population of oligonucleotides includes at least 10% (such as at least 20%, 30%, 40%, 50%, 60%, 70%, 80%, 85%, 90%, 95%, or 99%) of the 6 nucleotide sequences set forth in SEQ ID NOS:1-933.

Optionally, a spacer portion is located between the defined sequence portion and the hybridizing portion. The spacer portion is typically from 1 to 12 nucleotides long (e.g., from 1 to 6 nucleotides long) and can include any combination of nucleotides. The spacer portion can, for example, be composed of a random selection of nucleotides. All or part of the spacer portion may, or may not, hybridize to the same target nucleic acid sequence as the hybridizing portion. If all, or part, of the spacer portion hybridizes to the same target nucleic acid sequence as the hybridizing portion, then the effect is to enhance the efficiency of cDNA synthesis primed by the oligonucleotide that includes the hybridizing portion and the hybridizing spacer portion.

Hybridization conditions. In the practice of the present invention, a subpopulation of oligonucleotides is selected from a population of oligonucleotides based on the ability of the members of the subpopulation of oligonucleotides to hybridize, under defined conditions, to a first subpopulation of a target nucleic acid population, but not hybridize under the same defined conditions to a second subpopulation of the target nucleic acid population. The defined hybridization conditions permit the oligonucleotides to specifically hybridize to all nucleic acid molecules that are present in the sample except for globin mRNAs or ribosomal RNAs. Typically, hybridization conditions are no more than 25° C. to 30° C. (for example, 10° C.) below the melting temperature (Tm) of the native duplex. Tm for nucleic acid molecules greater than about 100 bases can be calculated by the formula Tm=81.5+0.41% (G+C)−log(Na+), wherein (G+C) is the guanosine and cytosine content of the nucleic acid molecule. For oligonucleotide molecules less than 100 bases in length, exemplary hybridization conditions are 5 to 10° C. below Tm. On average, the Tm of a short oligonucleotide duplex is reduced by approximately (500/oligonucleotide length)° C. In some embodiments of the present invention, the hybridization temperature is in the range of from 40° C. to 50° C. The appropriate hybridization conditions may also be identified empirically, without undue experimentation.

Amplification Conditions. In the practice of the present invention, the amplification of the first subpopulation of a target nucleic acid population occurs under defined amplification conditions. Hybridization conditions can be chosen as described, supra. Typically, the defined amplification conditions include first strand cDNA synthesis using a reverse transcriptase enzyme. The reverse transcription reaction is performed in the presence of defined concentrations of deoxynucleoside triphosphates (dNTPs). In some embodiments, the dNTP concentration is in a range from about 1000 to about 2000 microMolar in order to enrich the amplified product for target genes, as described in Examples 5-9.

Composition and synthesis of oligonucleotides. An oligonucleotide primer useful in the practice of the present invention can be DNA, RNA, PNA, chimeric mixtures, or derivatives or modified versions thereof, so long as it is still capable of priming the desired reaction. The oligonucleotide primer can be modified at the base moiety, sugar moiety, or phosphate backbone, and may include other appending groups or labels, so long as it is still capable of priming the desired amplification reaction.

For example, an oligonucleotide primer may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5N-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N2-carboxypropyl)uracil, and 2,6-diaminopurine.

Again by way of example, an oligonucleotide primer can include at least one modified sugar moiety selected from the group including, but not limited to, arabinose, 2-fluoroarabinose, xylulose, and hexose.

By way of further example, an oligonucleotide primer can include at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal, or analog thereof.

An oligonucleotide primer for use in the methods of the present invention may be derived by cleavage of a larger nucleic acid fragment using non-specific nucleic acid cleaving chemicals or enzymes, or site-specific restriction endonucleases, or by synthesis by standard methods known in the art, for example, by use of an automated DNA synthesizer (such as are commercially available from Biosearch, Applied Biosystems, etc.) and standard phosphoramidite chemistry. As examples, phosphorothioate oligonucleotides may be synthesized by the method of Stein et al. (Nucl. Acids Res. 16:3209-3221, 1988) and methylphosphonate oligonucleotides can be prepared by use of controlled pore glass polymer supports (Sarin et al., Proc. Natl. Acad. Sci. U.S.A. 85:7448-7451, 1988).

Once the desired oligonucleotide is synthesized, it is cleaved from the solid support on which it was synthesized, and treated, by methods known in the art, to remove any protecting groups present. The oligonucleotide may then be purified by any method known in the art, including extraction and gel purification. The concentration and purity of the oligonucleotide may be determined by examining an oligonucleotide that has been separated on an acrylamide gel, or by measuring the optical density at 260 nm in a spectrophotometer.

The methods of this aspect of the invention can be used, for example, to selectively amplify coding regions of mRNAs, introns, alternatively spliced forms of a gene, and non-coding RNAs that regulate gene expression.

In another aspect, the present invention provides populations of oligonucleotides comprising at least 10% (such as at least 20%, 30%, 40%, 50%, 60%, 70%, 80%, 85%, 90%, 95%, or 99%) of the nucleic acid sequences set forth in SEQ ID NOS:1-933. These oligonucleotides (SEQ ID NOS:1-933) can be used, for example, to prime the synthesis of cDNA molecules complementary to mRNA molecules isolated from mammalian blood without priming the synthesis of cDNA molecules complementary to globin mRNA or ribosomal RNA molecules. Indeed, these oligonucleotides (SEQ ID NOS:1-933) can be used, for example, to prime the synthesis of cDNA using any population of mRNA molecules as templates, without amplifying a significant amount of globin mRNAs or ribosomal RNAs. For example, the present invention provides populations of oligonucleotides wherein a defined sequence portion, such as a transcriptional promoter, such as the T7 promoter (SEQ ID NO:934), is located 5′ to a member of the population of oligonucleotides having the sequences set forth in SEQ ID NOS:1-933. Thus, in some embodiments, the present invention provides populations of oligonucleotides wherein each oligonucleotide consists of the T7 promoter (SEQ ID NO:934) located 5′ to a different member of the population of oligonucleotides having the sequences set forth in SEQ ID NOS:1-933. In some embodiments, the population of oligonucleotides includes at least 10% (such as 20%, 30%, 40%, 50%, 60%, 70%, 80%, 90%, 95%, or 99%) of the 6 nucleotide sequences set forth in SEQ ID NOS:1-933.

In another aspect, the present invention provides a reagent for selectively amplifying a target population of nucleic acid molecules. The reagent can be used, for example, to prime the synthesis of cDNA molecules complementary to mRNA molecules isolated from mammalian blood cells without priming the synthesis of cDNA molecules complementary to globin mRNA or ribosomal RNA molecules. The reagent of the present invention comprises a population of oligonucleotides comprising at least 10% of the nucleic acid sequences set forth in SEQ ID NOS:1-933. In some embodiments, the present invention provides a reagent comprising a population of oligonucleotides that includes at least 10% (such as 20%, 30%, 40%, 50%, 60%, 70%, 80%, 85%, 90%, 95%, or 99%) of the 6 nucleotide sequences set forth in SEQ ID NOS:1-933. In some embodiments, the population of oligonucleotides is selected to hybridize to substantially all nucleic acid molecules that are present in a sample except for globin mRNAs or ribosomal RNAs. In other embodiments, the population of oligonucleotides is selected to hybridize to a subset of nucleic acid molecules that are present in a sample, wherein the subset of nucleic acid molecules does not include globin mRNAs or ribosomal RNAs.

In another embodiment, the present invention provides a reagent that comprises a population of oligonucleotides wherein a defined sequence portion comprising a transcriptional promoter, such as the T7 promoter, is located 5′ to a member of the population of oligonucleotides having the sequences set forth in SEQ ID NOS:1-933. Thus, in some embodiments, the present invention provides a reagent comprising populations of oligonucleotides wherein each oligonucleotide consists of the T7 promoter (SEQ ID NO:934) located 5′ to a different member of the population of oligonucleotides having the sequences set forth in SEQ ID NOS:1-933.

In another embodiment, the present invention provides a reagent that comprises a population of oligonucleotides wherein a defined sequence portion comprising a primer binding site is located 5′ to a member of the population of oligonucleotides having the sequences set forth in SEQ ID NOS:1-933. Thus, in some embodiments, the present invention provides a reagent comprising populations of oligonucleotides wherein each oligonucleotide consists of the primer binding site (SEQ ID. NO:956) located 5′ to a different member of the population of oligonucleotides having the sequences set forth in SEQ ID NOS:1-933. The reagent of the present invention can be provided as an aqueous solution, or an aqueous solution with the water removed, or a lyophilized solid.

In a further embodiment, the reagent of the present invention may include one or more of the following components for the production of double-stranded cDNA: a reverse transcriptase, a DNA polymerase, a DNA ligase, a RNase H enzyme, a Tris buffer, a potassium salt, a magnesium salt, an ammonium salt, a reducing agent, deoxynucleoside triphosphates (dNTPs), [beta]-nicotinamide adenine dinucleotide (β-NAD+), and a ribonuclease inhibitor. For example, the reagent may include components optimized for first strand cDNA synthesis, such as a reverse transcriptase with reduced RNase H activity and increased thermal stability (e.g., SuperScript™ III Reverse Transcriptase, Invitrogen), and a final concentration of dNTPs in the range of from 50 to 5000 microMolar, or more preferably in the range of from 1000 to 2000 microMolar.

In another aspect, the present invention provides kits for selectively amplifying a target population of nucleic acid molecules. In some embodiments, the kits comprise a reagent that comprises a population of oligonucleotides wherein a defined sequence portion, such as a transcriptional promoter, (e.g., the T7 promoter), is located 5′ to a member of the population of oligonucleotides having the sequences set forth in SEQ ID NOS:1-933. Thus, in some embodiments, the present invention provides kits containing a reagent comprising populations of oligonucleotides wherein each oligonucleotide consists of the T7 promoter (SEQ ID NO:934) located 5′ to a different member of the population of oligonucleotides having the sequences set forth in SEQ ID NOS:1-933. The kits according to this embodiment are useful for producing in vitro transcribed RNA from cDNA generated using the not-so-random primers of the invention.

In some embodiments, the kits contain a reagent comprising populations of oligonucleotides wherein each oligonucleotide consists of a defined sequence suitable for use as a primer binding site located 5′ to a different member of the population of oligonucleotides having the sequences set forth in SEQ ID NOS:1-933. The kits according to this embodiment are useful for producing double-stranded DNA generated from PCR amplification of cDNA generated using the not-so-random primers of the invention.

The kits of the invention may be designed to detect any subpopulation of a target nucleic acid population, for example, all mRNAs expressed in a cell or tissue except for the most abundantly expressed mRNAs, in accordance with the methods described herein. Nonlimiting examples of mammalian mRNA target molecules include all or substantially all of the mRNA molecules from mammalian blood cells. Nonlimiting examples of exemplary oligonucleotide primers include SEQ ID NOS:1-933. Nonlimiting examples of the transcription promoter are set forth as SEQ ID NOS:934-936. Nonlimiting examples of primer binding regions are set forth as SEQ ID NO:946, 955, and 956.

The spacer portion may include any combination of nucleotides, including nucleotides that hybridize to the target mRNA.

In certain embodiments, the kit comprises a reagent comprising oligonucleotide primers with hybridizing portions of 6, 7, or 8 nucleotides.

In certain embodiments, the kit comprises a reagent comprising a population of oligonucleotide primers that may be used to detect a plurality of mammalian mRNA targets.

In certain embodiments, the kit comprises oligonucleotides that hybridize in the temperature range of from 40° C. to 50° C.

In another embodiment, the kit comprises a subpopulation of oligonucleotides that do not detect rRNA or globin mRNA. Exemplary oligonucleotides for use in accordance with this embodiment of the kit are provided in SEQ ID NOS:1-933.

In some embodiments, the kits comprises a reagent comprising a population of oligonucleotides comprising at least 10% (such as at least 20%, 30%, 40%, 50%, 60%, 70%, 80%, 85%, 90%, 95%, or 99%) of the 6 nucleotide sequences set forth in SEQ ID NOS:1-933.

In certain embodiments, the kit includes oligonucleotides wherein the transcription promoter comprises the T7 promoter (SEQ ID NO:934), the SP6 promoter (SEQ ID NO:935), or the T3 promoter (SEQ ID NO:936).

In another embodiment, the kit may comprise oligonucleotides with a spacer portion of from 1 to 12 nucleotides that comprises any combination of nucleotides.

In some embodiments of the present invention, the kit may further comprise one or more of the following components for the production of cDNA—a reverse transcriptase enzyme, a DNA polymerase enzyme, a DNA ligase enzyme, a RNase H enzyme, a Tris buffer, a potassium salt (e.g., potassium chloride), a magnesium salt (e.g., magnesium chloride), an ammonium salt (e.g., ammonium sulfate), a reducing agent (e.g., dithiothreitol), deoxynucleoside triphosphates (dNTPs), [beta]-nicotinamide adenine dinucleotide (β-NAD+), and a ribonuclease inhibitor. For example, the kit may include components optimized for first strand cDNA synthesis, such as a reverse transcriptase with reduced RNase H activity and increased thermal stability (e.g., SuperScript™ III Reverse Transcriptase, Invitrogen), and a dNTP stock solution to provide a final concentration of dNTPs in the range of from 50 to 5,000 microMolar, or more preferably in the range of from 1000 to 2000 microMolar.

In various embodiments, the kit may include a detection reagent such as SYBR green dye or BEBO dye that preferentially or exclusively binds to double stranded DNA during a PCR amplification step. In other embodiments, the kit may include a forward and/or reverse primer that includes a fluorophore and quencher to measure the amount of the PCR amplification products.

A kit of the invention can also provide reagents for in vitro transcription of the amplified cDNAs. For example, in some embodiments, the kit may further include one or more of the following components—a RNA polymerase enzyme, an IPPase (Inositol polyphosphate 1-phosphatase) enzyme, a transcription buffer, a Tris buffer, a sodium salt (e.g., sodium chloride), a magnesium salt (e.g., magnesium chloride), spermidine, a reducing agent (e.g., dithiothreitol), nucleoside triphosphates (ATP, CTP, GTP, UTP), and amino-allyl-UTP.

In another embodiment, the kit may include reagents for labeling the in vitro transcription products with Cy3 or Cy5 dye for use in hybridizing the labeled cDNA samples to microarrays.

The kit optionally includes instructions for using the kit in the selective amplification of mRNA targets. The kit can also be optionally provided with instructions for in vitro transcription of the amplified cDNA molecules, and with instructions for labeling and hybridizing the in vitro transcription products to microarrays.

In another aspect, the present invention provides methods of selectively amplifying a target population of nucleic acid molecules to generate amplified RNA molecules. The method comprises: (a) providing a population of oligonucleotides, wherein each oligonucleotide comprises a hybridizing portion and transcriptional promoter portion located 5′ to the hybridizing portion, wherein the hybridizing portion is a member of the population of oligonucleotides comprising SEQ ID NOS:1-933, (b) annealing the population of oligonucleotides to a sample comprising mRNA isolated from a mammalian subject, (c) synthesizing cDNA from the mRNA using a reverse transcriptase enzyme, (d) synthesizing double stranded cDNA using a DNA polymerase; and (e) transcribing the double-stranded cDNA into RNA using an RNA polymerase that binds to the transcriptional promoter portion of each oligonucleotide to generate amplified RNA. FIG. 1C shows a representative embodiment of the methods according to this aspect of the invention. The methods and reagents described herein are useful in the practice of this aspect of the invention.

In another aspect, the present invention provides methods of selectively amplifying a target population of nucleic acid molecules to generate amplified DNA molecules. The method comprises: (a) providing a first population of oligonucleotides, wherein each oligonucleotide comprises a hybridizing portion and a first PCR primer binding site located 5′ to the hybridizing portion, wherein the hybridizing portion is a member of the population of oligonucleotides comprising SEQ ID NOS:1-933, (b) annealing the population of oligonucleotides to a sample comprising mRNA isolated from a mammalian subject, (c) synthesizing cDNA from the mRNA using a reverse transcriptase enzyme, (d) synthesizing double stranded cDNA using a DNA polymerase a second population of oligonucleotides, wherein each oligonucleotide comprises a random hybridizing portion and a second PCR binding site located 5′ to the hybridizing portion, and (e) PCR amplifying the double stranded cDNA using thermostable DNA polymerase, a first PCR primer that binds to the first PCR primer binding site and a second PCR primer that binds to the second PCR primer binding site to generate amplified double stranded DNA. FIG. 1D illustrates a representative embodiment of the methods of this aspect of the invention.

In accordance with this aspect of the invention, any DNA-dependent DNA polymerase may be utilized to synthesize second-strand DNA molecules from the first strand cDNA. For example, the Klenow fragment of DNA Polymerase I can be utilized to synthesize the second-strand DNA molecules. The synthesis of second-strand DNA molecules is primed using a second population of oligonucleotides comprising a hybridizing portion consisting of from 6 to 9 random nucleotides and further comprising a defined sequence portion 5′ to the hybridizing portion. The defined sequence portion may include any suitable sequence, provided that the sequence differs from the defined sequence contained in the first population of oligonucleotides. Depending on the choice of primer sequence, these defined sequence portions can be used, for example, to selectively direct DNA-dependent RNA synthesis from the second DNA molecule and/or to amplify the double-stranded cDNA template via DNA-dependent DNA synthesis.

Purification of double-stranded DNA molecules: Synthesis of the second DNA molecules yields a population of double-stranded DNA molecules wherein the first DNA molecules are hybridized to the second DNA molecules, as shown in FIG. 1D. Typically, the double-stranded DNA molecules are purified to remove substantially all nucleic acid molecules shorter than 50 base pairs, including all, or substantially all (i.e., typically more than 99%), of the second primers. Preferably, the purification method selectively purifies DNA molecules that are substantially double-stranded and removes substantially all unpaired, single-stranded nucleic acid molecules, such as single-stranded primers. Purification can be achieved by any art-recognized means, such as by elution through a size-fractionation column. The purified, second DNA molecules can then, for example, be precipitated and redissolved in a suitable buffer for the next step of the methods of this aspect of the invention.

Amplification of the double-stranded DNA molecules. In the practice of the methods of this aspect of the invention, the double-stranded DNA molecules are utilized as templates that are enzymatically amplified using the polymerase chain reaction. Any suitable primers can be used to prime the polymerase chain reaction. Typically two primers are used, one primer hybridizes to the defined portion of the first primer sequence (or to the complement thereof); and the other primer hybridizes to the defined portion of the second primer sequence (or to the complement thereof).

PCR Amplification Conditions. In general, the greater the number of amplification cycles during the polymerase chain reaction, the greater the amount of amplified DNA that is obtained. On the other hand, too many amplification cycles may result in randomly biased amplification of the double-stranded DNA. Thus, in some embodiments, a desirable number of amplification cycles is between 5 and 40 amplification cycles, such as from five to 35, such as from 10 to 30 amplification cycles.

With regard to temperature conditions, typically a cycle comprises a melting temperature, such as 95° C.; an annealing temperature, which varies from about 40° C. to 70° C.; and an elongation temperature, which is typically about 72° C. With regard to the annealing temperature, in some embodiments the annealing temperature is from about 55° C. to 65° C., more preferably about 60° C.

In one embodiment, amplification conditions for use in this aspect of the invention comprise 10 cycles of (95° C., 30 sec; 60° C., 30 sec; 72° C., 60 sec), then 20 cycles of (95° C., 30 sec; 60° C., 30 sec, 72° C., 60 sec (+10 sec added to the elongation step with each cycle)).

With regard to PCR reaction components for use in the methods of this aspect of the invention, dNTPs are typically present in the reaction in a range from 50 μM to 2000 μM dNTPs, and more preferably from 800 to 1000 μM. MgCl₂ is typically present in the reaction in a range from 0.25 mM to 10 mM, and more preferably about 4 mM. The forward and reverse PCR primers are typically present in the reaction from about 50 nM to 2000 nM, and more preferably present at a concentration of about 1000 nM.

DNA Labeling. Optionally, the amplified DNA molecules can be labeled with a dye molecule to facilitate use as a probe in a hybridization experiment, such as a probe used to screen a DNA chip. Any suitable dye molecules can be utilized, such as fluorophores and chemiluminescers. An exemplary method for attaching the dye molecules to the amplified DNA molecules is provided in EXAMPLE 15.

The methods according this aspect of the invention may be used, for example, for transcriptome profiling in a biological sample containing total RNA, such as whole blood. In some embodiments, the amplified DNA produced in accordance with the methods of this aspect of the invention is labeled for use in gene expression experiments, thereby providing a hybridization based reagent that typically produces a lower level of background than amplified RNA generated from NSR-primed cDNA.

In some embodiments of this aspect of the invention, the defined sequence portion of the first and/or second primer binding regions further includes one or more restriction enzyme sites, thereby generating a population of amplified double-stranded DNA products having one or more restriction enzyme sites flanking the amplified portions. These amplified products may be used directly for sequence analysis, or may be released by digestion with restriction enzymes and subcloned into any desired vector, such as an expression vector for further analysis.

The following examples merely illustrate the best mode now contemplated for practicing the invention, but should not be construed to limit the invention.

### Example 1

This Example describes the selection of a population of 933 6-mer oligonucleotides (SEQ ID NOS:1-933) that hybridizes to all, or substantially all, mRNA molecules expressed in blood cells, but that does not hybridize to globin mRNA or to ribosomal RNA.

All 4096 possible 6-mer oligonucleotides were computed, wherein each nucleotide was A, T (or U), C, or G. The reverse complement of each 6-mer oligonucleotide was compared to the nucleotide sequences of 18S and 28S rRNAs, and to the nucleotide sequences of the following six hemoglobin genes, selected based on their high level of expression in blood samples:

TABLE 1

| | NCBI Reference Sequence |
|---|---|
| Gene Symbol | Transcript Identifier |
| HBA1 | NM_000558.3 |
| HBA2 | NM_000517.3 |
| HBB | NM_000518.4 |
| HBD | NM_000519.2 |
| HBG1 | NM_000559.2 |
| HBG2 | NM_000184.2 |

Reverse-complement 6-mer oligonucleotides having perfect matches to any of the eight transcript sequences were eliminated. The reverse complements of 933 6-mers (SEQ ID NOS:1-933) did not perfectly match any portion of the globin or rRNA transcripts. The 933 6-mer oligonucleotides (SEQ ID NOS:1-933) that do not have a perfect match to any portion of the globin or rRNA genes are referred to as “Not-So-Random” (“NSR”) primers.

### Example 2

This Example shows that the population of oligonucleotides having the nucleic acid sequences set forth in SEQ ID NOS:1-933 hybridize to every 4 to 5 nucleotides on a nucleic acid sequence within the RefSeq database accessible at the website of the National Center for Biotechnology Information (NCBI), U.S. National Library of Medicine, 8600 Rockville Pike, Bethesda, Md. 20894, U.S.A. NCBI's reference sequence transcript database (RefSeq) contains what is considered a gold-standard of human protein coding transcripts.

Random 6-mers (N6) can anneal at every nucleotide position on a transcript sequence from the RefSeq database (represented as “nucleotide sequence”), as shown in FIG. 1A. After subtracting out the 6-mers whose reverse complements are a perfect match to ribosomal RNAs and globin mRNAs, the remaining NSR oligonucleotides (SEQ ID NOS:1-933) show a perfect match to every 4 to 5 nucleotides on nucleic acid sequences within the RefSeq database (represented as “nucleotide sequence”), as shown in FIG. 1B. As shown in FIG. 2, NSR oligonucleotide binding sites are not present in the hemoglobin genes (represented as “A” in FIG. 2). One atypical gene family (represented as “B” in FIG. 2) consisting of 3 genes (LCE1A, CLE1D, LCE1F) contains only four NSR 6-mer binding sites per 100 nucleotides). However, RefSeq transcripts typically have anywhere from 5 to 30 NSR oligonucleotide (SEQ ID NOS:1-933) binding sites per 100 nucleotides, with most transcripts having 15-20 NSR binding sites per 100 nucleotides (represented as “C” in FIG. 2).

Thus, the population of 933 6-mers (SEQ ID NOS:1-933) is capable of amplifying all transcripts except 18S, 28S, and hemoglobin transcripts.

### Example 3

This Example shows that PCR amplification of an actin reporter mRNA using the 933 6-mers (SEQ ID NOS:1-933) (that each have the T7 promoter (SEQ ID NO:934) covalently attached at the 5′ end) selectively reduces priming of globin mRNA and rRNA.

Total RNA was extracted from individual donors of human whole blood using the PAXgene Blood RNA Kit (Qiagen, Inc., Valencia, Calif.) according to the manufacturer's instructions. MMLV (Moloney Murine Leukemia Virus) reverse transcriptase (Epicentre Biotechnologies, Madison, Wis.) was used to synthesize cDNA from 100 ng of template RNA with 5 μM 6-mers (SEQ ID NOS:1-933) (T7-NSR6) or random 9-mers with the T7 promoter covalently attached at the 5′ end (T7-N9). Prior to reverse transcription, 5 μL of water containing primers and template were denatured at 65° C. for 5 min, snap cooled at 4° C. and equilibrated to 23° C. For reverse transcription, 5 μL of RT master mix containing 1 μl of water, 2 μl of 5× First Strand Buffer (250 mM Tris-HCl, pH 8.3; 375 mM KCl; 15 mM MgCl₂; Invitrogen Corporation, Carlsbad, Calif.), 0.5 μl of 100 mM DTT, 0.5 μl of 10 mM dNTPs and 1.0 μl of MMLV reverse transcriptase (50 units/μl) was added to the sample mix. The 10 μl reaction was incubated at 40° C. for 120 min, 95° C. for 5 min, cooled to room temperature, and diluted 5-fold with water.

Following reverse transcription, duplicate measurements of 2 μl of cDNA were made in 10 μl final reaction volumes by quantitative PCR (qPCR) in a 384-well optical PCR plate using a 7900 HT PCR instrument (Applied Biosystems, Foster City, Calif.). qPCR was performed using ABI TaqMan® assays (eukaryotic 18S rRNA, ABI Catalog Hs99999901 s1; human beta actin, ABI Catalog No. 4310881E; human beta hemoglobin, ABI Catalog No. Hs00747223_g1; ABI Assay-by-Design alpha globin; (fwd 5′-GCACGCGCACAAGCT-3′ (SEQ ID NO:937), reverse 5′-GGGTCACCAGCAGGCA-3′ (SEQ ID NO:938), FAM probe 5′-ACTTCAAGCTCCTAAGCCAC-3′ (SEQ ID NO:939)) using the manufacturer's recommended conditions. Following PCR, the results table was exported to Excel (Microsoft Corp., Redmond, Wash.) and quantitative analysis for samples was regressed from the raw data (abundance=10^{[(Ct-5)/-3.4]}).

Reverse transcription of human whole blood total RNA with the 933 NSR 6-mers (SEQ ID NOS:1-933) resulted in a significant reduction of globin and rRNA content in cDNA as determined by qPCR. Specifically, T7-NSR 6 primed cDNA showed reduced abundance of alpha globin (58% reduction), beta globin (85% reduction) and 18S rRNA (75% reduction) as compared to cDNA generated with random 9-mers (T7-N9).

As shown below in TABLE 2, T7-NSR6 primed cDNA showed reduced abundance of beta globin (HBB). (94% reduction), and 18S rRNA (91% reduction), as compared to cDNA generated with random 9-mers. In contrast, beta actin cDNA levels for the two primer pools (T7-N9 and T7-NSR6) were comparable after amplification of RNA, a result that was confirmed using three independent donors (data not shown).

TABLE 2
Subtractive Amplification of total RNA from whole blood.

| | microarray signal | | |
|---|---|---|---|
| | hemoglobin | ribosomal | Actin |
| Primer | mRNA (HBB) | 18S rRNA | mRNA |
| T7-dT | not determined | 6,332,672 | not |
| | | | determined |
| T7-N9 | 2,072,315 | 2,627,237 | 188 |
| NSR-6-mer | 111,954 | 215,776 | 102 |

### Example 4

This Example shows that the 933 6-mers (SEQ ID NOS:1-933) (that each have the T7 promoter (SEQ ID NO:934) covalently attached at the ˜5′ end) prime the amplification of a substantial fraction of the transcriptome.

To assess cDNA complexity, 933 6-mers (SEQ ID NOS:1-933) were used to amplify total RNA from two diverse cell lines (Jurkat and K562) using a two-step reverse transcription and in vitro transcription (IVT) approach. The amplification products were labeled and hybridized to a DNA microarray containing probes for ˜5000 human transcripts known to be expressed in either Jurkat or K562 cells. Nearly 82% (4244/5198) of the transcripts reported above background signal intensity (log intensity ≧−1.5) in either cell line. Moreover, the Jurkat/K562 expression profile from RNA amplified using the 933 6-mers (SEQ ID NOS:1-933) showed a high degree of similarity to a control profile amplified from random-primed Jurkat and K562 mRNA (for probes with a p-value <0.01 in either experiment; correlation coefficient=0.72).

The amplification experiment was conducted as follows. Total RNA and mRNA were obtained from Ambion, Inc. (Austin, Tex.) for the cell lines Jurkat (T lymphocyte, ATCC No. TIB-152) and K562 (chronic myelogenous leukemia, ATCC No. CCL-243). A two-step amplification approach using reverse transcription and in vitro transcription was used to generate amplified RNA (aRNA) for microarray hybridizations. NSR cDNA was synthesized from 1 μg of total RNA and 5 μM primer in a 20 μL reaction volume as described in Example 3, but with a prolonged incubation step at 40° C. for 6 hrs. cDNA was synthesized from 100 ng of mRNA using 10 μM random 9-mer in a 20 μL reaction volume as described in Example 3. The 933 6-mers (SEQ ID NOS:1-933) and the random 9-mers were covalently linked to the T7 promoter sequence (SEQ ID NO:934) at the 5′ end.

For in vitro transcription, the 20 μL RT reaction was added to 60 μL of IVT pre-mix containing 16 μL of 5× Transcription Buffer (0.2 M Tris-HCl, pH 7.5, 50 mM NaCl, 30 mM MgCl₂, and 10 mM spermidine; Epicentre Biotechnologies, Madison, Wis.), 6 μL of 100 mM DTT, 3.3 μL of 200 mM MgCl₂, 8 μL of NTP (25 mM ATP, 25 mM CTP, 25 mM GTP, 6 mM UTP), 2 μL of 75 mM amino allyl-UTP, 0.6 μL of IPPase (2 U/μL), 0.08 μL, of T7 RNA polymerase (2.5 kU/μL), 24 μL of water and incubated for 16 hrs at 40° C., 5 min at 70° C., and cooled to room temperature. The final product was coupled to Cy3 or Cy5 dye in 1 M bicarbonate buffer for 1 h. Reactions were finished with the addition of 4 M hydroxylamine followed by purification with RNAeasy Spin Columns. The percentage dye incorporation and total cDNA yield were determined spectrophotometrically. Pairs of Cy3/Cy5-labeled cDNA samples were combined and hybridized to microarrays as described previously (Roberts et al., “Signaling and Circuitry of Multiple MAPK Pathways Revealed by a Matrix of Global Gene Expression Profiles,” Science 287:873-880, 2000). Arrays were hybridized for 48 hrs then washed and scanned on Agilent Microarray Scanners.

Custom high density oligonucleotide arrays were obtained from Agilent Technologies (Palo Alto, Calif.). A total of 5198 transcripts were chosen for array probe design from previously described experiments (Hughes et al., “Expression profiling using microarrays fabricated by an ink-jet oligonucleotide synthesizer,” Nat. Biotechnol. 19(4):342-347, 2001). Probe sequences (60-mers) were selected on the basis of nucleotide composition and cross-hybridization potential, and positioned at least 500 bp upstream of the known polyadenylation site for each transcript. Data analysis was carried out as previously described (Hughes et al., supra).

### Example 5

This example shows that increasing the concentration of deoxyribonucleoside triphosphates (dNTPs) in the first strand synthesis reaction increases the amplification of IGF1R and GAPDH reporter mRNA using NSR primers (comprising SEQ ID NOS:1-933) (that each have the T7 promoter (SEQ ID NO:934) covalently attached at the 5′ end), relative to globin mRNA and rRNA.

Methods:

NSR primers were used in this Example as follows:

The 933 6-mers (SEQ ID NOS:1-933) plus a single random nucleotide inserted between the 6-mer sequence and the T7 promoter (SEQ ID NO:934), referred to as “NSR7.”

Total RNA was extracted from human whole blood as described in Example 3. MMLV reverse transcriptase (SuperScript III™ (“SSIII”), Invitrogen Corporation, Carlsbad, Calif.) was used to synthesize cDNA from 200 ng of template RNA with 10 μM 6-mers (SEQ ID NOS:1-933) or random 8-mers (N8). First strand synthesis was performed as described in Example 3, except that the reaction volume was 20 μL, 2 μL SSIII reverse transcriptase (200 units/μl) was added to the reaction mix, and the concentration of dNTPs varied from 500 to 5,000 μM. The reaction was incubated at 40° C. for 120 min, 70° C. for 15 min, cooled to room temperature, and diluted two-fold with 10 mM Tris pH 7.6, 0.1 mM EDTA.

Following reverse transcription, qPCR was performed as described in Example 3, except that ABI TaqMan assays for human insulin growth factor one receptor (IGFR1R) (ABI Catalog #Hs00181385) and human glyceraldehyde-3-phosphate dehydrogenase (GAPDH) (ABI Catalog #43100884E-0507028) were substituted for human beta actin as reporters. Following PCR, the results table was exported to Excel and quantitative analysis for samples was regressed from the raw data as described in Example 3.

Reverse transcription of human whole blood total RNA with the NSR7 primers (comprising SEQ ID NOS:1-933) and increasing concentrations of dNTPs resulted in increased specificity of the NSR7-primed reaction. The data from two independent experiments is shown in Tables 1-5 below. The raw abundance values, expressed as 10 raised to the (Cts×log 10(½)+10) power (Tables 3 and 4), were normalized to the highest N8 value for a given target gene (Tables 5 and 6).

TABLE 3
Raw abundance values vs. dNTP concentration. Values expressed as
10 raised to the (Cts × log10(½) + 10) power.

| | | [dNTPs] μM | | | | | | | |
|---|---|---|---|---|---|---|---|---|---|
| Target Gene | Primer set | 500 | 1000 | 1500 | 2000 | 2500 | 3000 | 4000 | 5000 |
| IGF1R | NSR | 30 | 36 | 28 | 21 | 14 | 9 | 2 | 0 |
| | N8 | 82 | 68 | 76 | 61 | 49 | 29 | 15 | 5 |
| GAPDH | NSR | 428 | 519 | 399 | 361 | 281 | 263 | 56 | 1 |
| | N8 | 1009 | 857 | 1000 | 649 | 567 | 302 | 167 | 6 |
| 18S | NSR | 22576 | 19812 | 7255 | 1843 | 408 | 30 | 0 | 0 |
| | N8 | 377536 | 466002 | 584331 | 291882 | 103635 | 9602 | 26 | 0 |
| 28S | NSR | 162393 | 27816 | 9910 | 1516 | 243 | 40 | 2 | 2 |
| | N8 | 613070 | 148408 | 110193 | 59667 | 12571 | 2558 | 33 | 1 |
| HBA | NSR | 1689 | 1450 | 759 | 607 | 341 | 226 | 168 | 37 |
| | N8 | 8963 | 7271 | 11116 | 12151 | 7088 | 6578 | 1815 | 1241 |
| HBB | NSR | 1473 | 1128 | 588 | 528 | 423 | 373 | 270 | 0 |
| | N8 | 2838 | 3574 | 6312 | 4588 | 3276 | 2513 | 962 | 255 |

TABLE 4
Raw abundance values vs. dNTP concentration. Values expressed as
10 raised to the (Cts × log10(½) + 10) power.

| Target | Primer | [dNTPs] μM | | | | | | | |
|---|---|---|---|---|---|---|---|---|---|
| Gene | set | 500 | 1000 | 1500 | 2000 | 2500 | 3000 | 4000 | 5000 |
| IGF1R | NSR | 201 | 177 | 154 | 110 | 71 | 45 | 7 | 1 |
| | N8 | 495 | 230 | 259 | 211 | 215 | 152 | 73 | 25 |
| GAPDH | NSR | 288 | 405 | 350 | 270 | 229 | 221 | 61 | 7 |
| | N8 | 530 | 490 | 424 | 359 | 329 | 265 | 93 | 16 |
| 18S | NSR | 605722 | 393759 | 170366 | 60940 | 26468 | 4076 | 8 | 1 |
| | N8 | 3338794 | 2685092 | 2257580 | 1149677 | 589425 | 46427 | 137 | 0 |
| 28S | NSR | 622085 | 71670 | 34636 | 19885 | 6064 | 581 | 4 | 0 |
| | N8 | 1346589 | 1310318 | 1744481 | 970265 | 412149 | 63991 | 1236 | 1 |
| HBA | NSR | 5047 | 2114 | 1125 | 1395 | 884 | 531 | 235 | 80 |
| | N8 | 44790 | 35305 | 95774 | 158007 | 447716 | 540325 | 214874 | 73825 |
| HBB | NSR | 5420 | 1937 | 1484 | 1774 | 1672 | 1577 | 2448 | 4182 |
| | N8 | 29146 | 24501 | 22769 | 24853 | 21620 | 11380 | 1710 | 477 |

TABLE 5
Abundance values normalized to N8.

| | | [dNTPs] μM | | | | | | | |
|---|---|---|---|---|---|---|---|---|---|
| Target RNA | Primer set | 500 | 1000 | 1500 | 2000 | 2500 | 3000 | 4000 | 5000 |
| IGF1R | NSR | 44 | 53 | 41 | 31 | 20 | 13 | 2 | 0 |
| | N8 | 120 | 100 | 112 | 90 | 72 | 43 | 22 | 7 |
| GAPDH | NSR | 50 | 61 | 47 | 42 | 33 | 31 | 7 | 0 |
| | N8 | 118 | 100 | 117 | 76 | 66 | 35 | 20 | 1 |
| 18S | NSR | 5 | 4 | 2 | 0 | 0 | 0 | 0 | 0 |
| | N8 | 81 | 100 | 125 | 63 | 22 | 2 | 0 | 0 |
| 28S | NSR | 109 | 19 | 7 | 1 | 0 | 0 | 0 | 0 |
| | N8 | 413 | 100 | 74 | 40 | 8 | 2 | 0 | 0 |
| HBA | NSR | 23 | 20 | 10 | 8 | 5 | 3 | 2 | 1 |
| | N8 | 123 | 100 | 153 | 167 | 97 | 90 | 25 | 17 |
| HBB | NSR | 41 | 32 | 16 | 15 | 12 | 10 | 8 | 0 |
| | N8 | 79 | 100 | 177 | 128 | 92 | 70 | 27 | 7 |

TABLE 6
Abundance normalized to N8.

| | | [dNTPs] | | | | | | | |
|---|---|---|---|---|---|---|---|---|---|
| | | 500 | 1000 | 1500 | 2000 | 2500 | 3000 | 4000 | 5000 |
| IGF1R | NSR | 41 | 36 | 31 | 22 | 14 | 9 | 1 | 0 |
| | N8 | 100 | 46 | 52 | 43 | 44 | 31 | 15 | 5 |
| GAPDH | NSR | 54 | 77 | 66 | 51 | 43 | 42 | 11 | 1 |
| | N8 | 100 | 93 | 80 | 68 | 62 | 50 | 17 | 3 |
| 18S | NSR | 18 | 12 | 5 | 2 | 1 | 0 | 0 | 0 |
| | N8 | 100 | 80 | 68 | 34 | 18 | 1 | 0 | 0 |
| 28S | NSR | 46 | 5 | 3 | 1 | 0 | 0 | 0 | 0 |
| | N8 | 100 | 97 | 130 | 72 | 31 | 5 | 0 | 0 |
| HBA | NSR | 11 | 5 | 3 | 3 | 2 | 1 | 1 | 0 |
| | N8 | 100 | 79 | 214 | 353 | 1000 | 1206 | 480 | 165 |
| HBB | NSR | 19 | 7 | 5 | 6 | 6 | 5 | 8 | 14 |
| | N8 | 100 | 84 | 78 | 85 | 74 | 39 | 6 | 2 |

The ratios of reporters to backgrounds (reporter/background), which is the ratio of IGF1R and GAPDH reporters to 18S rRNA, 28S rRNA, alpha globin (HBA) and beta globin (HBB) background values, were then calculated for the NSR7 and N8 primer pools. As shown in Tables 7 and 8, the NSR7-primed reporter/background ratio increased dramatically with increasing dNTP concentrations.

TABLE 7
Ratios of Reporters to Background.

| Reporter/ | Primer | [dNTPs] μM | | | | | | | |
|---|---|---|---|---|---|---|---|---|---|
| Background | set | 500 | 1000 | 1500 | 2000 | 2500 | 3000 | 4000 | 5000 |
| IGF1R/18S | NSR | 9 | 12 | 26 | 78 | 228 | 1963 | 39729 | 2974 |
| | N8 | 1 | 1 | 1 | 1 | 3 | 21 | 3872 | 72108 |
| IGF1R/28S | NSR | 0 | 3 | 6 | 30 | 122 | 463 | 1701 | 101 |
| | N8 | 0 | 1 | 2 | 2 | 9 | 25 | 986 | 13040 |
| IGF1R/HBA | NSR | 2 | 3 | 4 | 4 | 4 | 4 | 1 | 0 |
| | N8 | 1 | 1 | 1 | 1 | 1 | 0 | 1 | 0 |
| IGF1R/HBB | NSR | 1 | 2 | 2 | 2 | 2 | 1 | 0 | 12 |
| | N8 | 2 | 1 | 1 | 1 | 1 | 1 | 1 | 1 |
| GAPDH/18S | NSR | 10 | 14 | 30 | 107 | 374 | 4786 | 111747 | 1211 |
| | N8 | 1 | 1 | 1 | 1 | 3 | 17 | 3436 | 7399 |
| GAPDH/28S | NSR | 0 | 3 | 7 | 41 | 200 | 1129 | 4783 | 41 |
| | N8 | 0 | 1 | 2 | 2 | 8 | 20 | 875 | 1338 |
| GAPDH/HBA | NSR | 2 | 3 | 4 | 5 | 7 | 10 | 3 | 0 |
| | N8 | 1 | 1 | 1 | 0 | 1 | 0 | 1 | 0 |
| GAPDH/HBB | NSR | 1 | 2 | 3 | 3 | 3 | 3 | 1 | 5 |
| | N8 | 1 | 1 | 1 | 1 | 1 | 1 | 1 | 0 |

TABLE 8
Ratio of reporters to backgrounds.

| | | [dNTPs] μM | | | | | | | |
|---|---|---|---|---|---|---|---|---|---|
| | | 500 | 1000 | 1500 | 2000 | 2500 | 3000 | 4000 | 5000 |
| IGF1R/18S | NSR | 2 | 3 | 6 | 12 | 18 | 74 | 6090 | 5194 |
| | N8 | 1 | 1 | 1 | 1 | 2 | 22 | 3594 | 2356335 |
| IGF1R/28S | NSR | 1 | 7 | 12 | 15 | 32 | 211 | 5197 | 6174 |
| | N8 | 1 | 0 | 0 | 1 | 1 | 6 | 160 | 51368 |
| IGF1R/HBA | NSR | 4 | 8 | 12 | 7 | 7 | 8 | 3 | 1 |
| | N8 | 1 | 1 | 0 | 0 | 0 | 0 | 0 | 0 |
| IGF1R/HBB | NSR | 2 | 5 | 6 | 4 | 3 | 2 | 0 | 0 |
| | N8 | 1 | 1 | 1 | 0 | 1 | 1 | 3 | 3 |
| GAPDH/18S | NSR | 3 | 6 | 13 | 28 | 55 | 342 | 49034 | 42162 |
| | N8 | 1 | 1 | 1 | 2 | 4 | 36 | 4275 | 1374635 |
| GAPDH/28S | NSR | 1 | 14 | 26 | 35 | 96 | 967 | 41850 | 50116 |
| | N8 | 1 | 1 | 1 | 1 | 2 | 11 | 191 | 29967 |
| GAPDH/HBA | NSR | 5 | 16 | 26 | 16 | 22 | 35 | 22 | 7 |
| | N8 | 1 | 1 | 0 | 0 | 0 | 0 | 0 | 0 |
| GAPDH/HBB | NSR | 3 | 12 | 13 | 8 | 8 | 8 | 1 | 0 |
| | N8 | 1 | 1 | 1 | 1 | 1 | 1 | 3 | 2 |

The ratios of ratios is a comparison of reporter to background ratios for the NSR7 and N8 primer pools, and measures the specificity of the NSR-primed reactions. As shown in Table 9 and Table 10, the ratios of ratios increased dramatically at higher dNTP concentrations.

TABLE 9
Ratios of Ratios (Reporter/Background versus NSR7/N8).

| Reporter/ | Primer | [dNTPs] μM | | | | | | | |
|---|---|---|---|---|---|---|---|---|---|
| Background | Ratio | 500 | 1000 | 1500 | 2000 | 2500 | 3000 | 4000 | 5000 |
| IGF1R/18S | NSR/N8 | 6 | 12 | 29 | 54 | 70 | 94 | 10 | 0 |
| IGF1R/28S | NSR/N8 | 1 | 3 | 4 | 14 | 14 | 19 | 2 | 0 |
| IGF1R/HBA | NSR/N8 | 2 | 3 | 5 | 7 | 6 | 9 | 1 | 1 |
| IGF1R/HBB | NSR/N8 | 1 | 2 | 4 | 3 | 2 | 2 | 0 | 13 |
| GAPDH/18S | NSR/N8 | 7 | 14 | 32 | 88 | 126 | 280 | 33 | 0 |
| GAPDH/28S | NSR/N8 | 2 | 3 | 4 | 22 | 26 | 55 | 5 | 0 |
| GAPDH/HBA | NSR/N8 | 2 | 3 | 6 | 11 | 10 | 25 | 4 | 3 |
| GAPDH/HBB | NSR/N8 | 1 | 2 | 4 | 5 | 4 | 6 | 1 | 51 |

TABLE 10
Ratios of Ratios (Reporter/Background versus NSR/N8).

| | [dNTPs] μM | | | | | | | |
|---|---|---|---|---|---|---|---|---|
| | 500 | 1000 | 1500 | 2000 | 2500 | 3000 | 4000 | 5000 |
| IGF1R/18S NSR/N8 | 2 | 5 | 8 | 10 | 7 | 3 | 2 | 0 |
| IGF1R/28S NSR/N8 | 1 | 14 | 30 | 26 | 22 | 32 | 32 | 0 |
| IGF1R/HBA NSR/N8 | 4 | 13 | 51 | 59 | 167 | 300 | 88 | 28 |
| IGF1R/HBB NSR/N8 | 2 | 10 | 9 | 7 | 4 | 2 | 0 | 0 |
| GAPDH/18S NSR/N8 | 3 | 6 | 11 | 14 | 16 | 10 | 11 | 0 |
| GAPDH/28S NSR/N8 | 1 | 15 | 42 | 37 | 47 | 92 | 220 | 2 |
| GAPDH/HBA NSR/N8 | 5 | 14 | 70 | 85 | 353 | 850 | 599 | 392 |
| GAPDH/HBB NSR/N8 | 3 | 10 | 13 | 11 | 9 | 6 | 0 | 0 |

The yield ratio measures the output of cDNA conversion primed by NSR7 versus N8, expressed as a percentage (NSR divided by N8). As shown in Table 11 and Table 12, the NSR to N8 yield ratio was substantially higher for the IGF1R and GAPDH reporters than for the rRNA and globin mRNA. The ratio was relatively constant across dNTP concentrations for the IGF1R and GAPDH reporters, but decreased with increasing dNTP concentrations for the rRNA and globin genes, consistent with enrichment of reporters at higher dNTP concentrations.

TABLE 11
Yield Ratios of NSR/N8.

| | | [dNTPs] μM | | | | | | | |
|---|---|---|---|---|---|---|---|---|---|
| Gene | | 500 | 1000 | 1500 | 2000 | 2500 | 3000 | 4000 | 5000 |
| IGF1R | NSR/N8 | 37 | 53 | 36 | 34 | 28 | 29 | 11 | 2 |
| GAPDH | NSR/N8 | 42 | 61 | 40 | 56 | 49 | 87 | 34 | 9 |
| 18S | NSR/N8 | 6 | 4 | 1 | 1 | 0 | 0 | 1 | 57 |
| 28S | NSR/N8 | 26 | 19 | 9 | 3 | 2 | 2 | 6 | 306 |
| HBA | NSR/N8 | 19 | 20 | 7 | 5 | 5 | 3 | 9 | 3 |
| HBB | NSR/N8 | 52 | 32 | 9 | 11 | 13 | 15 | 28 | 0 |

TABLE 12
Yields of NSR/N8.

| | [dNTPs] μM | | | | | | | |
|---|---|---|---|---|---|---|---|---|
| | 500 | 1000 | 1500 | 2000 | 2500 | 3000 | 4000 | 5000 |
| IGF1R | 41 | 77 | 59 | 52 | 33 | 29 | 10 | 3 |
| NSR/N8 | | | | | | | | |
| GAPDH | 54 | 83 | 83 | 75 | 70 | 83 | 66 | 43 |
| NSR/N8 | | | | | | | | |
| 18S NSR/N8 | 18 | 15 | 8 | 5 | 4 | 9 | 6 | 1393 |
| 28S NSR/N8 | 46 | 5 | 2 | 2 | 1 | 1 | 0 | 26 |
| HBA NSR/N8 | 11 | 6 | 1 | 1 | 0 | 0 | 0 | 0 |
| HBB NSR/N8 | 19 | 8 | 7 | 7 | 8 | 14 | 143 | 877 |

In summary, this example shows that the absolute yields of cDNA primed with the NSR7 primers (comprising SEQ ID NOS:1-933) were highest at low dNTP concentrations, whereas the specificity relative to random 8-mers increased at higher dNTP concentrations. Based on this data, it appears that the optimal concentration of dNTPs to achieve high yield and high specificity is in the range of about 1000 to about 2000 μM dNTPs.

### Example 6

This Example shows that NSR primers (comprising SEQ ID NOS:1-933), that each have the T7 promoter (SEQ ID NO:934) covalently attached at the 5′ end, maintain specificity when low amounts of template RNA is reverse transcribed and amplified by in vitro transcription (WT).

In initial experiments, it was determined that the NSR7-primed specificity was lost during the second strand synthesis and IVT amplification steps when low amounts of template RNA were used. The IVT amplification step is required to produce enough RNA for the microarray experiments disclosed above.

It was observed that the NSR7-primed specificity from low amounts of template RNA was improved by using the SuperScript™ RNA Amplification System (Invitrogen, Catalog No. L1016-01), as follows:

Methods: First strand cDNA synthesis was performed as described in Example 5 using 1000 μM dNTPs, and 50 ng, 100 ng, and 200 ng of whole blood total RNA template with the NSR7 primer pool. 100 ng of RNA template was used with random 7-mers that each have the T7 promoter (SEQ ID NO:934) covalently attached at the 5′ end (T7-N7) and random 8-mer (N8) primers. For second strand cDNA synthesis, 20 μL of the first strand reaction product was added to 130 μL of second strand mix containing 91 μL water, 30 μL 5× Second-Strand Reaction Buffer (100 mM Tris-HCl (pH 6.9), 450 mM KCl, 23 mM MgCl₂, 0.75 mM β-NAD⁺, 50 mM (NH₄)₂SO₄) 3 μL 10 mM dNTPs, 4 μL DNA Polymerase I (10 units/μl), 1 μL DNA ligase (10 units/μl) and 1 μL RNase H (2 units/μl). The reaction was incubated at 16° C. for 120 min. The double stranded cDNA was purified using the Spin Cartridge and buffers supplied in the kit (Invitrogen), and the volume of the eluted cDNA was reduced to less than 20 μL by centrifugation under vacuum with low to moderate heat.

The double stranded cDNA template was amplified by in vitro transcription using T7 polymerase to transcribe antisense RNA (aRNA) complementary to the original mRNA targets. For IVT, the volume of the purified cDNA was adjusted to 23 μL with water, added to 17 μL of IVT reaction mix containing 4 μL 10×T7 Reaction Buffer (Invitrogen), 7 μL of T7 Enzyme Mix (includes T7 RNA polymerase), 8 μL NTP (25 mM each of ATP, CTP, GTP, and UTP), and incubated at 37° C. for 6 to 16 hours. The resulting amplified aRNA was purified using the Spin Cartridges and aRNA Binding and Wash Buffers supplied by Invitrogen, and the yield of aRNA was quantified using Nanodrop Technologies.

The aRNA was reverse transcribed with random 8-mers (N8) to produce single stranded cDNA. For reverse transcription, 500 ng or 1000 ng of aRNA was added to 20 μl of RT mix containing 4 μl 5× first strand buffer (250 mM Tris-HCl, pH 8.3; 375 mM KCl; 15 mM MgCl₂), 2 μl 10 mM dNTPs, 1 μl 100 mM DTT, 1 μl RNAseOUT™ (40 units/μl), 2 μl SuperScript III Reverse Transcriptase (200 units/μl), and incubated at 40° C. for 60 min, 70° C. for 10 min, and cooled on ice.

Following reverse transcription, qPCR was performed as described in Example 3 using ABI Taqman® assays for IGFR1 (catalog #Hs00181385), GAPDH (catalog #43100884E-0507028), GUSB (catalog #4310888E), ACTIN (catalog #4310881E), eukaryotic 18SrRNA (catalog #Hs99999901s1), and human hemoglobin beta (HBB) (catalog #Hs00747223 g1)

For eukaryotic 28S rRNA and human hemoglobin alpha (HBA) custom primers and probes (containing a 5′-FAM (6-carboxyfluorescein) reporter dye and a no fluorescent quencher (NFQ) at the 3′ end of the probe) were used as follows:

Eukaryotic 28S rRNA:

(SEQ ID NO:940)

| | |
|---|---|
| Forward primer: | 5′ ACGGTGGCCATGGAAGTC 3′; |

(SEQ ID NO:941)

| | |
|---|---|
| Reverse primer: | 5′ TCGGCAGGTGAGTTGTTACAC 3′; |

(SEQ ID NO:942)

| | |
|---|---|
| FAM Probe: | 5′ ACTCCTTAGCGGATTCC 3′ |

Human hemoglobin alpha (HBA):

(SEQ ID NO:943)

| | |
|---|---|
| Forward primer: | 5′ GCACGCGCACAAGCT 3′ |

(SEQ ID NO:944)

| | |
|---|---|
| Reverse primer: | 5′ GGGTCACCAGCAGGCA 3′; |

(SEQ ID NO:945)

| | |
|---|---|
| FAM Probe | 5′ ACTTCAAGCTCCTAAGCCAC 3′ |

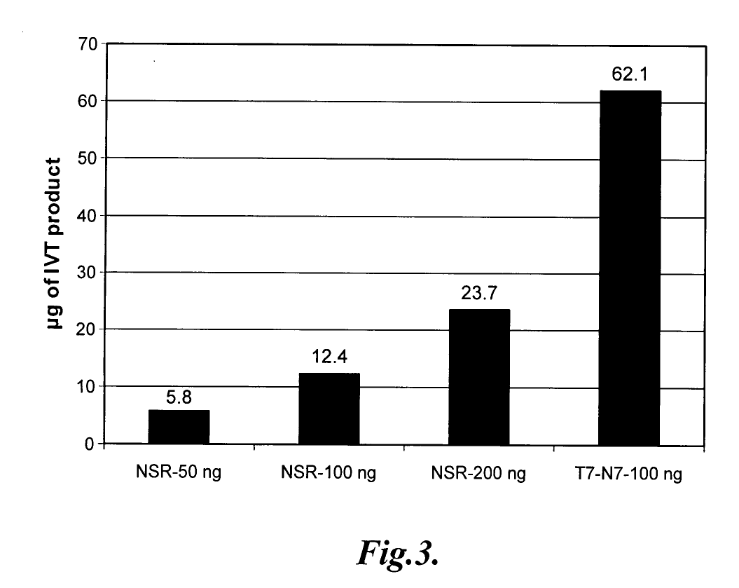

Reverse transcription of human whole blood total RNA with the NSR7 (comprising SEQ ID NOS:1-933) followed by IVT produced high yields of aRNA that were directly proportional to the amount of input RNA template as shown in FIG. 3.

Quantitative PCR (qPCR) analysis of the cDNA and reverse transcribed IVT products (IVT-RT) from varying amounts of RNA template is shown in Table 13 (raw Ct values) and Table 14 (absolute abundance). Primer pool indicates the primers used for first strand cDNA synthesis. Product indicates either the cDNA or IVT-RT product was the template for qPCR.

TABLE 13
Raw Ct values for
SSIII reverse transcribed cDNA (left side columns, cDNA)
followed by IVT-RT (right side columns, IVT).

| | RNA template (ng) | | | | | | | | | |
|---|---|---|---|---|---|---|---|---|---|---|
| | 50 | 100 | 200 | 100 | 100 | 100 | 50 | 100 | 200 | 100 |
| | Primer pool | | | | | | | | | |
| | NSR | NSR | NSR | T7-N7 | N8 | no primer | NSR | NSR | NSR | T7-N7 |
| | product | | | | | | | | | |
| | cDNA | cDNA | cDNA | cDNA | cDNA | cDNA | IVT | IVT | IVT | IVT |
| IGF1 ® | 29 | 27 | 27 | 26 | 25 | 33 | 21 | 21 | 21 | 23 |
| GAPDH | 29 | 28 | 26 | 27 | 26 | 31 | 21 | 21 | 21 | 23 |
| GUSB | 34 | 33 | 32 | 32 | 29 | 33 | 25 | 25 | 25 | 26 |
| ACTIN | 29 | 28 | 26 | 26 | 23 | 31 | 21 | 22 | 21 | 22 |
| 18S | 19 | 17 | 17 | 16 | 15 | 21 | 12 | 12 | 12 | 13 |
| 28S | 17 | 15 | 14 | 15 | 14 | 16 | 14 | 14 | 14 | 14 |
| HBA | 21 | 20 | 19 | 18 | 16 | 24 | 17 | 16 | 16 | 17 |
| HBB | 21 | 20 | 19 | 18 | 17 | 23 | 17 | 16 | 16 | 16 |

TABLE 14
Absolute abundance values of
SSIII reverse transcribed cDNA (left side columns, cDNA)
followed by IVT-RT (right side columns, IVT).

| | RNA template (ng) | | | | | | | | | |
|---|---|---|---|---|---|---|---|---|---|---|
| | 50 | 100 | 200 | 100 | 100 | 100 | 50 | 100 | 200 | 100 |
| | Primer pool | | | | | | | | | |
| | NSR | NSR | NSR | T7-N7 | N8 | no primer | NSR | NSR | NSR | T7-N7 |
| | Product | | | | | | | | | |
| | cDNA | cDNA | cDNA | cDNA | cDNA | cDNA | IVT | IVT | IVT | IVT |
| IGF1 ® | 22 | 53 | 97 | 111 | 226 | 1 | 3641 | 3824 | 4344 | 1676 |
| GAPDH | 13 | 26 | 138 | 59 | 178 | 5 | 4290 | 4966 | 5375 | 1043 |
| GUSB | 0 | 1 | 3 | 2 | 21 | 1 | 328 | 354 | 384 | 200 |
| ACTIN | 16 | 42 | 123 | 120 | 891 | 4 | 3465 | 3154 | 3518 | 2319 |
| 18S | 14933 | 61683 | 75134 | 124343 | 237330 | 3556 | 2071662 | 2497065 | 2881157 | 1015695 |
| 28S | 99635 | 233965 | 434367 | 339446 | 780775 | 141364 | 499476 | 633708 | 663349 | 816928 |
| HBA | 5441 | 9387 | 17205 | 42639 | 115379 | 564 | 101594 | 132380 | 119639 | 72935 |
| HBB | 4712 | 9505 | 19323 | 36727 | 66940 | 967 | 97505 | 119805 | 127175 | 169351 |

The ratio of reporter to background RNA is shown in Table 15. There was little substantial NSR7 specificity relative to T7-N7 observed during the initial first strand cDNA step (left columns). Importantly, the ratio of reporter to background RNAs increased going from the first strand synthesis step to the IVT step (right columns) for both the NSR7 and T7-N7 primers (the data was normalized to N8).

TABLE 15
Reporter/background ratios for varying amounts
of RNA template after first strand cDNA synthesis (left columns)
and IVT-RT (right columns) (normalized to N8).

| | RNA template (ng) | | | | | | | | | |
|---|---|---|---|---|---|---|---|---|---|---|
| | 50 | 100 | 200 | 100 | 100 | 100 | 50 | 100 | 200 | 100 |
| | Primer pool | | | | | | | | | |
| | NSR | NSR | NSR | T7-N7 | N8 | no primer | NSR | NSR | NSR | T7-N7 |
| | product | | | | | | | | | |
| | cDNA | cDNA | cDNA | cDNA | cDNA | cDNA | IVT | IVT | IVT | IVT |
| IGF1R/18S | 1.55 | 0.90 | 1.36 | 0.94 | 1.00 | 0.36 | 1.84 | 1.60 | 1.58 | 1.73 |
| IGF1R/28S | 0.76 | 0.78 | 0.77 | 1.13 | 1.00 | 0.03 | 25.13 | 20.81 | 22.58 | 7.07 |
| IGF1R/HBA | 2.07 | 2.88 | 2.88 | 1.33 | 1.00 | 1.09 | 18.26 | 14.72 | 18.50 | 11.71 |
| IGF1R/HBB | 1.39 | 1.65 | 1.49 | 0.89 | 1.00 | 0.37 | 11.04 | 9.44 | 10.10 | 2.92 |
| GAPDH/18S | 1.19 | 0.57 | 2.45 | 0.64 | 1.00 | 1.96 | 2.76 | 2.65 | 2.49 | 1.37 |
| GAPDH/28S | 0.59 | 0.49 | 1.39 | 0.77 | 1.00 | 0.16 | 37.66 | 34.36 | 35.53 | 5.60 |
| GAPDH/HBA | 1.59 | 1.82 | 5.19 | 0.90 | 1.00 | 6.00 | 27.36 | 24.30 | 29.11 | 9.27 |
| GAPDH/HBB | 1.06 | 1.04 | 2.68 | 0.61 | 1.00 | 2.03 | 16.54 | 15.58 | 15.89 | 2.32 |
| GUSB/18S | 0.36 | 0.22 | 0.48 | 0.16 | 1.00 | 2.78 | 1.80 | 1.61 | 1.52 | 2.24 |
| GUSB/28S | 0.18 | 0.19 | 0.27 | 0.19 | 1.00 | 0.23 | 24.59 | 20.90 | 21.65 | 9.15 |
| GUSB/HBA | 0.48 | 0.69 | 1.02 | 0.22 | 1.00 | 8.53 | 17.86 | 14.78 | 17.74 | 15.14 |
| GUSB/HBB | 0.32 | 0.40 | 0.53 | 0.15 | 1.00 | 2.89 | 10.80 | 9.48 | 9.68 | 3.78 |
| ACTIN/18S | 0.28 | 0.18 | 0.44 | 0.26 | 1.00 | 0.33 | 0.45 | 0.34 | 0.33 | 0.61 |
| ACTIN/28S | 0.14 | 0.16 | 0.25 | 0.31 | 1.00 | 0.03 | 6.08 | 4.36 | 4.65 | 2.49 |
| ACTIN/HBA | 0.38 | 0.58 | 0.93 | 0.36 | 1.00 | 1.02 | 4.42 | 3.09 | 3.81 | 4.12 |
| ACTIN/HBB | 0.25 | 0.33 | 0.48 | 0.25 | 1.00 | 0.35 | 2.67 | 1.98 | 2.08 | 1.03 |

Quantitative PCR analysis of the IVT products generated from NSR7-primed cDNA showed that the composition of each sample was approximately the same at three different total RNA input concentrations (50 ng, 100 ng, and 200 ng). Composition refers to normalized yields of reporter and background IVT products. As shown in Table 16, the normalized yields were very similar for both the reporter and background genes across a 4-fold range of input RNA. This data suggests that the IVT products were amplified from the appropriate target RNA and the reaction was not overwhelmed with nonspecific transcript material.

TABLE 16
qPCR analysis of the NSR7 IVT-RT products
at varying RNA template amounts.

| | NSR-50 ng | NSR-100 ng | NSR-200 ng | average |
|---|---|---|---|---|
| IGF1R | 3641 | 3824 | 4344 | 3936 |
| GAPDH | 4290 | 4966 | 5375 | 4877 |
| GUSB | 328 | 354 | 384 | 355 |
| ACTIN | 3465 | 3154 | 3518 | 3379 |
| 18S | 2071662 | 2497065 | 2881157 | 2483294 |
| 28S | 499476 | 633708 | 663349 | 598844 |
| HBA | 101594 | 132380 | 119639 | 117871 |
| HBB | 97505 | 119805 | 127175 | 114828 |

### Example 7

This Example shows that NSR7 primers (comprising SEQ ID NOS:1-933), that each have the T7 promoter (SEQ ID NO:934) covalently attached at the 5′ end, coupled with high dNTP concentrations, generate first strand RT products with high specificity, and that these products can be amplified with the SuperScript™ RNA Amplification System (Invitrogen) to produce high yields of IVT product substantially enriched for target genes.

Methods:

400 ng of whole blood total RNA was reverse transcribed using the NSR7, T7-N7, and N8 primers in 401 reaction volume, as described in Example 5. 20 μl (200 ng whole blood total RNA) was used to assess IGF1R, GAPDH, rRNA and globin abundance by qPCR, and the best performing set of primers was then subjected to IVT. Two primer concentrations (10 μM and 25 μM) and two dNTP concentrations (1000 μM and 2500 μM) were evaluated. The raw Ct and absolute abundance values following first strand reverse transcription are shown in Table 17 and Table 18.

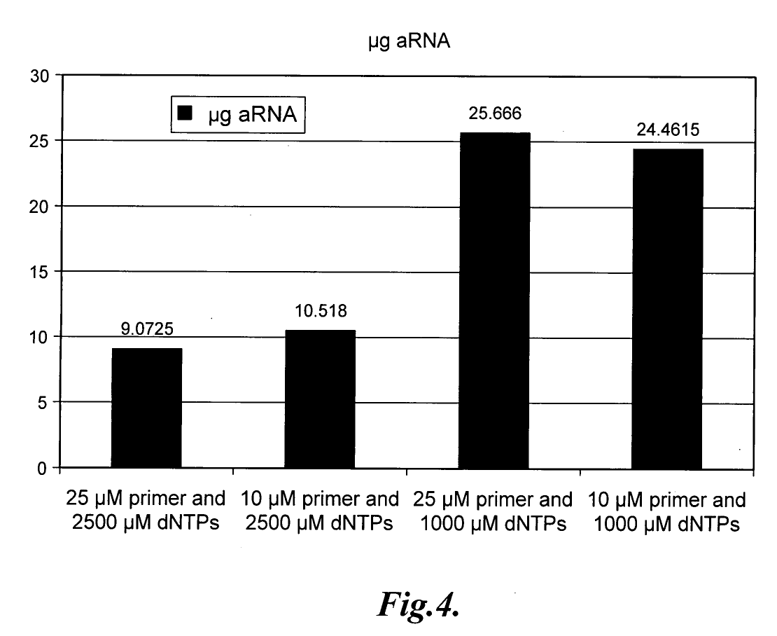

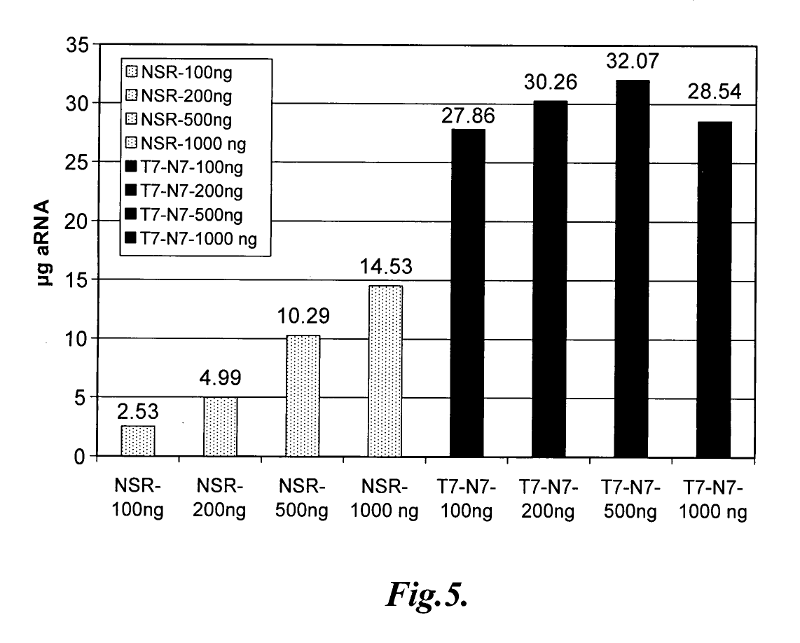

One μg of aRNA from the IVT reaction was converted back into cDNA by reverse transcription for qPCR analysis, as described in Example 6. FIG. 4 shows that the specific activity of the reporter genes IGF1R and GAPDH was increased in the presence of high dNTP concentrations, whereas the specific activity of background rRNA and globin mRNA was decreased in the presence of high dNTP concentrations.

The raw Ct and abundance values following qPCR of the IVT-RT reaction products is shown in Tables 19 and 20. The reporter to background ratios for first strand NSR7 and IVT-RT are shown in Table 21.

The ratio of ratios data allows a comparison of first strand synthesis yields with IVT yields following conversion to cDNA and qPCR. Comparison of reporter to background ratios and NSR7 to N8 ratios shows that increasing the dNTP concentration from 1000 mM to 2500 mM resulted in a pronounced increase in NSR7-mediated specificity, as shown in Table 22. The primer concentration had a less noticeable impact on the NSR7-mediated specificity. In addition, comparing the ratios of NSR7-primed cDNA synthesis yields to the IVT yields showed a dramatic relative reduction in rRNAs (18S and 28S), suggesting that the rRNA cDNAs observed in the first round of reverse transcription fail to be transcribed during IVT (Table 23). The relative reduction in background globin mRNA yields between first strand synthesis and IVT was not observed with the NSR7 primers. When first strand synthesis was primed with random 8-mers, the reporter genes IGF1R and GAPDH were enriched relative to both rRNA and globin mRNA following IVT (Table 24).

In summary, this Example shows that high dNTP concentrations in combination with the NSR7 primers (comprising SEQ ID NOS:1-933) generate first strand reverse transcription products with high NSR7 specificity. The first strand products can then be amplified using the SuperScript™ RNA Amplification System (Invitrogen) to produce high yields of IVT product that is substantially enriched for target genes.

TABLE 17
Raw Ct values from the first strand cDNA reactions.

| | NSR RT | | | | N8 RT | | | |
|---|---|---|---|---|---|---|---|---|
| | First strand Primer pool Primer concentration (mM) | | | | | | | |
| | 25 and | 10 and | 25 and | 10 and | 25 and | 10 and | 25 and | 10 and |
| | 2500 | 2500 | 1000 | 1000 | 2500 | 2500 | 1000 | 1000 |
| IGF1R | 27 | 27 | 26 | 26 | 25 | 26 | 25 | 25 |
| GAPDH | 24 | 24 | 24 | 24 | 23 | 23 | 23 | 23 |
| 18S | 18 | 18 | 14 | 14 | 13 | 14 | 12 | 12 |
| 28S | 20 | 20 | 15 | 15 | 13 | 14 | 12 | 12 |
| HBA | 22 | 23 | 19 | 19 | 16 | 16 | 17 | 17 |
| HBB | 23 | 24 | 21 | 21 | 16 | 16 | 16 | 16 |

Key to the table headings:
25 and 2500 means 25 μM primer and 2500 μM dNTPs;
10 and 2500 means 10 μM primer and 2500 μM dNTPs;
25 and 1000 means 25 μM primer and 1000 μM dNTPs;
10 and 1000 means 10 μM primer and 1000 μM dNTPs;

TABLE 18
Absolute abundance values from the first strand cDNA reactions.

| | NSR | | | | N8 | | | |
|---|---|---|---|---|---|---|---|---|
| Primer | 25 and | 10 and | 25 and | 10 and | 25 and | 10 and | 25 and | 10 and |
| pool | 2500 | 2500 | 1000 | 1000 | 2500 | 2500 | 1000 | 1000 |
| IGF1R | 98 | 80 | 171 | 176 | 242 | 190 | 409 | 363 |
| GAPDH | 613 | 624 | 501 | 740 | 1215 | 966 | 1504 | 1497 |
| 18S | 48666 | 45494 | 548592 | 467387 | 1168629 | 831556 | 2699215 | 2001591 |
| 28S | 9154 | 9053 | 289871 | 381748 | 950029 | 765345 | 2754112 | 2747920 |
| HBA | 1971 | 886 | 15374 | 14544 | 197577 | 158464 | 89286 | 54844 |
| HBB | 1136 | 424 | 5406 | 4152 | 152329 | 176372 | 155202 | 135652 |

TABLE 19
Raw Ct values for the IVT-RT reactions. NTC = No template control.

| | NSR | | | | |
|---|---|---|---|---|---|
| Primer | 25 | | | | |
| pool | and 2500 | 10 and 2500 | 25 and 1000 | 10 and 1000 | NTC |
| IGF1R | 22 | 21 | 22 | 22 | 40 |
| GAPDH | 21 | 20 | 22 | 21 | 40 |
| 18S | 19 | 17 | 15 | 14 | 40 |
| 28S | 21 | 20 | 16 | 16 | 36 |
| HBA | 18 | 17 | 17 | 17 | 40 |
| HBB | 20 | 19 | 19 | 18 | 40 |

TABLE 20
Absolute abundance values for the IVT-RT reactions.

| | NSR | | | |
|---|---|---|---|---|
| Primer pool | 25 and 2500 | 10 and 2500 | 25 and 1000 | 10 and 1000 |
| IGF1R | 3174 | 3642 | 2586 | 2190 |
| GAPDH | 5218 | 12093 | 2592 | 5350 |
| 18S | 25303 | 55054 | 295696 | 503082 |
| 28S | 6332 | 9545 | 125816 | 137191 |
| HBA | 49703 | 79131 | 55051 | 82026 |
| HBB | 10622 | 19903 | 26634 | 46817 |

TABLE 21
Reporter to background ratios (*1000) calculated
from the abundance data in Tables 17 and 19.

| | | NSR-IVT RT | | | |
|---|---|---|---|---|---|
| | | | 10 and | | 10 and |
| | | 25 and 2500 | 2500 | 25 and 1000 | 1000 |
| IGF1R/18S | IVT | 125 | 66 | 9 | 4 |
| | NSR7 | 2 | 2 | 0 | 0 |
| | N8 | 0 | 0 | 0 | 0 |
| IGF1R/28S | IVT | 501 | 382 | 21 | 16 |
| | NSR | 11 | 9 | 1 | 0 |
| | N8 | 0 | 0 | 0 | 0 |
| IGF1R/HBA | IVT | 64 | 46 | 47 | 27 |
| | NSR | 50 | 90 | 11 | 12 |
| | N8 | 1 | 1 | 5 | 7 |
| IGF1R/HBB | IVT | 299 | 183 | 97 | 47 |
| | NSR | 86 | 188 | 32 | 42 |
| | N8 | 2 | 1 | 3 | 3 |
| GAPDH/18S | IVT | 206 | 220 | 9 | 11 |
| | NSR | 13 | 14 | 1 | 2 |
| | N8 | 1 | 1 | 1 | 1 |
| GAPDH/28S | IVT | 824 | 1267 | 21 | 39 |
| | NSR | 67 | 69 | 2 | 2 |
| | N8 | 1 | 1 | 1 | 1 |
| GAPDH/HBA | IVT | 105 | 153 | 47 | 65 |
| | NSR | 311 | 704 | 33 | 51 |
| | N8 | 6 | 6 | 17 | 27 |
| GAPDH/HBB | IVT | 491 | 608 | 97 | 114 |
| | NSR | 540 | 1473 | 93 | 178 |
| | N8 | 8 | 5 | 10 | 11 |

TABLE 22
Ratios of ratios comparing NSR7 and N8 primed
first strand synthesis after IVT-RT.

| | NSR IVT RT | | | |
|---|---|---|---|---|
| | 25 and | | | 10 and |
| ratios of ratios | 2500 | 10 and 2500 | 25 and 1000 | 1000 |
| IGF1R/18S NSR/N8 | 10 | 8 | 2 | 2 |
| IGF1R/28S NSR/N8 | 42 | 35 | 4 | 3 |
| IGF1R/HBA NSR/N8 | 41 | 75 | 2 | 2 |
| IGF1R/HBB NSR/N8 | 54 | 174 | 12 | 16 |
| GAPDH/18S NSR/N8 | 12 | 12 | 2 | 2 |
| GAPDH/28S NSR/N8 | 52 | 55 | 3 | 4 |
| GAPDH/HBA NSR/N8 | 51 | 116 | 2 | 2 |
| GAPDH/HBB NSR/N8 | 68 | 269 | 10 | 16 |

TABLE 23
Ratios of ratios comparing NSR7 primed first strand synthesis
before and after IVT-RT.

| | NSR IVT RT | | | |
|---|---|---|---|---|
| | 25 and | | | 10 and |
| | 2500 | 10 and 2500 | 25 and 1000 | 1000 |
| IGF1R/18S IVT/NSR | 62 | 38 | 28 | 12 |
| IGF1R/28S IVT/NSR | 47 | 43 | 35 | 35 |
| IGF1R/HBA IVT/NSR | 1.3 | 0.5 | 4 | 2 |
| IGF1R/HBB IVT/NSR | 3 | 1.0 | 3 | 1.1 |
| GAPDH/18S IVT/NSR | 16 | 16 | 10 | 7 |
| GAPDH/28S IVT/NSR | 12 | 18 | 12 | 20 |
| GAPDH/HBA IVT/NSR | 0.3 | 0.2 | 1.4 | 1.3 |
| GAPDH/HBB IVT/NSR | 0.9 | 0.4 | 1.1 | 0.6 |

TABLE 24
Ratios of ratios comparing N8 primed first strand synthesis
before and after IVT-RT.

| | NSR-IVT RT | | | |
|---|---|---|---|---|
| | 25 and | | | 10 and |
| | 2500 | 10 and 2500 | 25 and 1000 | 1000 |
| IGF1R/18S IVT/N8 | 607 | 289 | 58 | 24 |
| IGF1R/28S IVT/N8 | 1971 | 1533 | 138 | 121 |
| IGF1R/HBA IVT/N8 | 52 | 38 | 10 | 4 |
| IGF1R/HBB IVT/N8 | 188 | 169 | 37 | 17 |
| GAPDH/18S IVT/N8 | 198 | 189 | 16 | 14 |
| GAPDH/28S IVT/N8 | 644 | 1004 | 38 | 72 |
| GAPDH/HBA IVT/N8 | 17 | 25 | 3 | 2 |
| GAPDH/HBB IVT/N8 | 62 | 111 | 10 | 10 |

### Example 8

This Example shows that selective enhancement of IGF1R and GAPDH reporter mRNAs relative to rRNA and globin mRNA using the NSR7 primers (comprising SEQ ID NOS:1-933), that each have the T7 promoter (SEQ ID NO:934) covalently attached at the 5′ end, with high concentrations of dNTPs occurs over a wide range of whole blood total RNA template concentrations.

Total human whole blood RNA was reverse transcribed and analyzed by qPCR as described in Example 5. The amount of template RNA was 20 ng, 50 ng, 100 ng, 500 ng, 1000 ng, 2000 ng, and 5000 ng. The data show that the NSR7 mediated enrichment of IGF1R and GAPDH reporters relative to rRNA and globin mRNA (as determined by the ratio of ratios for NSR7 versus N8) is observed at all template amounts (Table 25 and Table 26). The reporter/background ratio of ratios for NSR versus N8 increases at higher template amounts, but the overall yield of first strand cDNA product is compromised (Table 27). One microgram (1000 ng) of input template RNA represents an optimal compromise between cDNA yields and NSR7-mediated amplification specificity. At higher RNA template amounts (2000 ng and 5000 ng) the reverse transcription reaction failed to fully convert RNA into cDNA. The cDNA yields in Table 28 were determined by multiplying the absolute yields of product (from Table 29) by the dilution factor relative to 5000 ng of template RNA, then normalizing to the N8 yield from 20 ng of template RNA which consistently produced the highest yield values. The normalized cDNA yields of IGF1R and GAPDH were similar across all template amounts for both NSR7 and N8, with a decreased yield at the highest RNA template concentrations. In contrast, the yield of 18S, 28S, alpha globin, and beta globin products was substantially higher when primed with N8 than when primed with NSR7.

In summary, the “NSR effect,” defined as a high percentage of conversion by the NSR7 primers (comprising SEQ ID NOS:1-933) of reporter mRNA to cDNA relative to N8, but a low percentage of conversion by the NSR7 primers of background RNAs to cDNA relative to N8, is conserved across all template concentrations tested. This provides the advantage that reaction conditions do not need to be customized to template amounts.

TABLE 25
Ratio of ratios for reporter/background and NSR/N8
at different RNA template amounts.

| | | ng total RNA | | | | | | | |
|---|---|---|---|---|---|---|---|---|---|
| | | 5000 | 2000 | 1000 | 500 | 200 | 100 | 50 | 20 |
| IGF1R/18S | NSR/N8 | 26 | 30 | 25 | 16 | 10 | 8 | 7 | 11 |
| IGF1R/28S | NSR/N8 | 7 | 8 | 9 | 7 | 8 | 7 | 7 | 9 |
| IGF1R/HBA | NSR/N8 | 26 | 34 | 30 | 19 | 11 | 7 | 5 | 8 |
| IGF1R/HBB | NSR/N8 | 14 | 16 | 19 | 15 | 10 | 6 | 5 | 8 |
| GAPDH/18S | NSR/N8 | 40 | 44 | 37 | 24 | 13 | 10 | 9 | 11 |
| GAPDH/28S | NSR/N8 | 11 | 12 | 14 | 11 | 10 | 9 | 8 | 9 |
| GAPDH/HBA | NSR/N8 | 42 | 50 | 45 | 27 | 15 | 9 | 7 | 8 |
| GAPDH/HBB | NSR/N8 | 22 | 23 | 28 | 22 | 13 | 8 | 7 | 9 |

TABLE 26
Percent reduction of background RNAs for the NSR/N8 ratio
at different RNA template amounts.

| | ng total RNA | 5000 | 2000 | 1000 | 500 | 200 | 100 | 50 | 20 |
|---|---|---|---|---|---|---|---|---|---|
| IGF1R/18S | NSR/N8 | 96 | 97 | 96 | 94 | 90 | 87 | 86 | 91 |
| IGF1R/28S | NSR/N8 | 86 | 88 | 89 | 86 | 87 | 86 | 85 | 89 |
| IGF1R/HBA | NSR/N8 | 96 | 97 | 97 | 95 | 91 | 86 | 82 | 88 |
| IGF1R/HBB | NSR/N8 | 93 | 94 | 95 | 93 | 90 | 84 | 81 | 88 |
| GAPDH/18S | NSR/N8 | 98 | 98 | 97 | 96 | 93 | 90 | 89 | 91 |
| GAPDH/28S | NSR/N8 | 91 | 92 | 93 | 91 | 90 | 89 | 88 | 89 |
| GAPDH/HBA | NSR/N8 | 98 | 98 | 98 | 96 | 93 | 89 | 85 | 88 |
| GAPDH/HBB | NSR/N8 | 95 | 96 | 96 | 95 | 92 | 88 | 85 | 89 |

TABLE 27
cDNA yields normalized to N8.

| | ng | | | | | | | | |
|---|---|---|---|---|---|---|---|---|---|
| | total | | | | | | | | |
| | RNA | 5000 | 2000 | 1000 | 500 | 200 | 100 | 50 | 20 |
| IGF1R | NSR | 15 | 25 | 36 | 35 | 41 | 36 | 37 | 40 |
| | N8 | 33 | 54 | 70 | 82 | 90 | 90 | 96 | 100 |
| GAPDH | NSR | 18 | 32 | 43 | 43 | 48 | 43 | 40 | 42 |
| | N8 | 27 | 47 | 56 | 67 | 81 | 83 | 86 | 100 |
| 18S | NSR | 0 | 1 | 2 | 2 | 4 | 4 | 4 | 4 |
| | N8 | 18 | 62 | 97 | 93 | 93 | 86 | 84 | 100 |
| 28S | NSR | 1 | 2 | 3 | 4 | 5 | 5 | 5 | 5 |
| | N8 | 8 | 27 | 50 | 67 | 87 | 84 | 87 | 100 |
| HBA | NSR | 0 | 1 | 1 | 1 | 3 | 4 | 5 | 5 |
| | N8 | 16 | 41 | 63 | 63 | 74 | 72 | 72 | 100 |
| HBB | NSR | 0 | 1 | 1 | 2 | 4 | 5 | 6 | 5 |
| | N8 | 10 | 22 | 44 | 58 | 80 | 80 | 79 | 100 |

TABLE 28
Ratios of NSR synthesis to N8 synthesis across
all template conditions expressed as a % of N8.

| | ng total RNA | 5000 | 2000 | 1000 | 500 | 200 | 100 | 50 | 20 |
|---|---|---|---|---|---|---|---|---|---|
| IGF1R | NSR/N8 | 44 | 47 | 51 | 43 | 45 | 40 | 38 | 40 |
| GAPDH | NSR/N8 | 69 | 69 | 77 | 64 | 59 | 51 | 47 | 42 |
| 18S | NSR/N8 | 2 | 2 | 2 | 3 | 4 | 5 | 5 | 4 |
| 28S | NSR/N8 | 6 | 6 | 5 | 6 | 6 | 6 | 6 | 5 |
| HBA | NSR/N8 | 2 | 1 | 2 | 2 | 4 | 6 | 7 | 5 |
| HBB | NSR/N8 | 3 | 3 | 3 | 3 | 5 | 6 | 7 | 5 |

TABLE 29
qPCR of NSR and N8 primed first strand reverse transcription products
across a range of RNA template amounts. Raw Ct data.

| | ng total RNA | 5000 | 2000 | 1000 | 500 | 200 | 100 | 50 | 20 |
|---|---|---|---|---|---|---|---|---|---|
| IGF1R | NSR | 25.21 | 25.73 | 26.22 | 27.26 | 28.37 | 29.55 | 30.52 | 31.71 |
| | N8 | 24.02 | 24.65 | 25.26 | 26.04 | 27.22 | 28.22 | 29.13 | 30.40 |
| GAPDH | NSR | 21.05 | 21.55 | 22.14 | 23.15 | 24.30 | 25.47 | 26.55 | 27.83 |
| | N8 | 20.50 | 21.02 | 21.75 | 22.50 | 23.55 | 24.51 | 25.46 | 26.56 |
| 18S | NSR | 20.53 | 20.19 | 20.17 | 20.86 | 21.45 | 22.33 | 23.32 | 24.93 |
| | N8 | 14.65 | 14.20 | 14.56 | 15.61 | 16.94 | 18.05 | 19.08 | 20.16 |
| 28S | NSR | 19.21 | 18.96 | 19.12 | 19.60 | 20.57 | 21.63 | 22.57 | 24.02 |
| | N8 | 15.19 | 14.84 | 14.93 | 15.51 | 16.46 | 17.51 | 18.46 | 19.58 |
| HBA | NSR | 21.73 | 21.93 | 22.00 | 22.55 | 22.85 | 23.40 | 24.09 | 25.42 |
| | N8 | 15.81 | 15.74 | 16.13 | 17.12 | 18.22 | 19.26 | 20.26 | 21.11 |
| HBB | NSR | 21.30 | 21.50 | 21.65 | 22.17 | 22.35 | 22.90 | 23.75 | 25.31 |
| | N8 | 16.30 | 16.45 | 16.48 | 17.07 | 17.92 | 18.92 | 19.94 | 20.92 |

TABLE 30
qPCR of NSR and N8 primed first strand reverse transcription products
across a range of RNA template amounts. Absolute abundance data.

| | ng total RNA | 5000 | 2000 | 1000 | 500 | 200 | 100 | 50 | 20 |
|---|---|---|---|---|---|---|---|---|---|
| IGF1R | NSR | 257.04 | 179.85 | 127.89 | 62.30 | 28.88 | 12.70 | 6.51 | 2.85 |
| | N8 | 589.66 | 380.40 | 249.07 | 144.45 | 63.77 | 31.92 | 17.06 | 7.08 |
| GAPDH | NSR | 4615.40 | 3253.99 | 2167.62 | 1076.00 | 483.73 | 215.10 | 101.95 | 41.96 |
| | N8 | 6720.73 | 4697.05 | 2825.73 | 1691.11 | 816.01 | 419.57 | 217.38 | 100.91 |
| 18S | NSR | 6613.12 | 8365.89 | 8469.08 | 5242.78 | 3501.13 | 1893.76 | 954.04 | 313.53 |
| | N8 | 388405.53 | 531139.00 | 412768.53 | 199313.85 | 79602.24 | 36801.88 | 17994.25 | 8554.29 |
| 28S | NSR | 16479.87 | 19626.05 | 17496.69 | 12591.15 | 6419.05 | 3076.32 | 1602.46 | 589.43 |
| | N8 | 268154.87 | 339917.11 | 320454.27 | 214361.60 | 110770.23 | 53752.24 | 27678.38 | 12728.46 |
| HBA | NSR | 2869.10 | 2505.03 | 2381.69 | 1625.43 | 1326.05 | 902.22 | 558.24 | 222.34 |
| | N8 | 173954.32 | 182225.26 | 139416.35 | 70042.65 | 32706.21 | 15922.00 | 7984.30 | 4424.14 |
| HBB | NSR | 3868.82 | 3382.72 | 3033.17 | 2121.01 | 1869.38 | 1279.18 | 711.20 | 240.02 |
| | N8 | 123516.47 | 112017.08 | 109557.90 | 72684.35 | 40199.75 | 20201.73 | 9951.70 | 5035.32 |

### Example 9

This Example shows that the NSR7 primers (comprising SEQ ID NOS:1-933), that each have the T7 promoter (SEQ ID NO:934) covalently attached at the 5′ end, combined with high dNTP concentrations show increased specificity when compared to random 7-mers (T7-N7), and that this specificity is maintained across a range of RNA template amounts.

First-strand cDNA synthesis, second-strand synthesis, IVT, reverse transcription of the IVT, and qPCR of the aRNA were performed as described in Example 6. The RNA template amounts were 100 ng, 200 ng, 500 ng, and 1000 ng of whole blood total RNA. The reaction volumes were 40 μl, and the reactions were incubated at 40° C. for 180 min, 70° C. for 10 min, and chilled on ice. The dNTP concentration was 2000 μM. Second strand synthesis reaction was for 120 min at 16° C.

The data from the first-strand synthesis indicated that high dNTP concentrations result in increased levels of IGF1R and GAPDH reporters relative to rRNA and globin background genes when primed with the NSR7 pool, but not when primed with the T7-N7 pool (data normalized to N8) (Table 30). See FIG. 5. The data in Table 31 further shows that the enrichment of reporters relative to background was also observed following in vitro transcription of the NSR7-primed cDNA, the enrichment tended to be more pronounced for the ribosomal rRNAs than the globin mRNAs, and the enrichment was observed across a range of input RNA template amounts. The ratio of ratios shows that reporters are enriched relative to background when comparing NSR7 to T7-N7 for both the first strand and IVT reactions, and this effect was observed at all RNA input amounts tested (Table 32). Table 33 shows the raw qPCR data from the NSR7, T7-N7 and N8-primed first strand cDNA and IVT-RT reactions. Table 34 shows the raw abundance data from the NSR7, T7-N7 and N8-primed first strand cDNA and IVT-RT reactions.

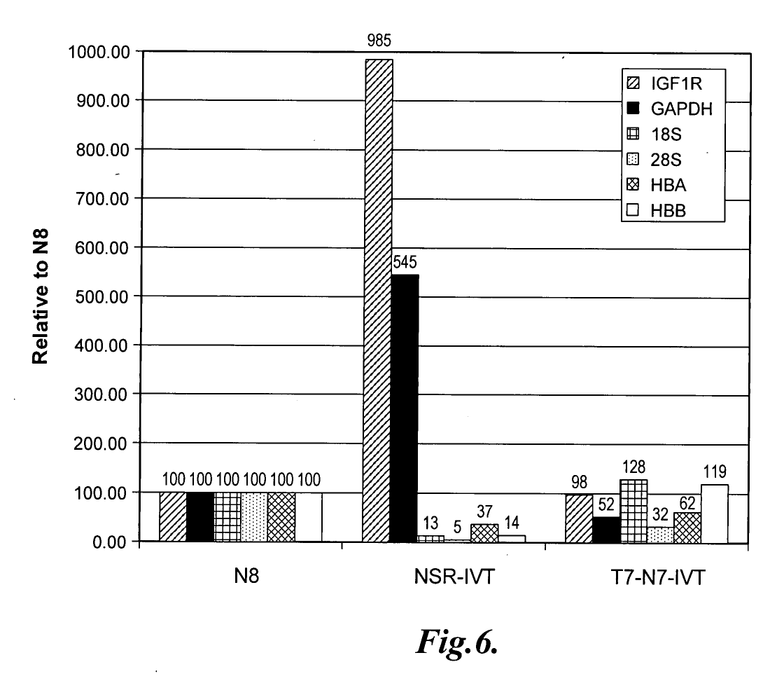

FIG. 5 is a histogram plot showing the yield of amplified RNA from T7-NSR primed and T7-N7 primed cDNA as a function of the amount of input RNA template. The amount of input RNA tested ranged from 100 ng, 200 ng, 500 ng, and 1000 ng. FIG. 6 is a histogram plot on a linear scale showing the relative composition of T7-NSR-primed and T7-N7 primed cDNA (normalized to N8) following amplification by in vitro transcription (IVT). As shown in FIGS. 5 and 6, the composition of the NSR-primed IVT product is substantially different than the composition of the N8-primed cDNA. As shown in FIG. 6, following IVT with the NSR primer pool, the IGF1R and GAPDH reporters increased and the rRNA and globin background decreased. In contrast, the IVT product from the T7-N7 primer pool was not substantially different than the starting material when normalized to N8-primed cDNA.

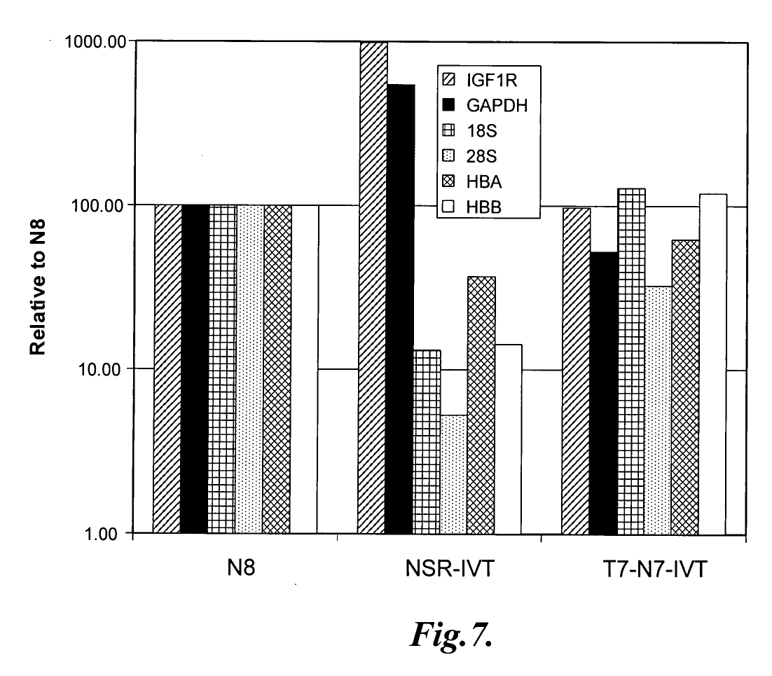

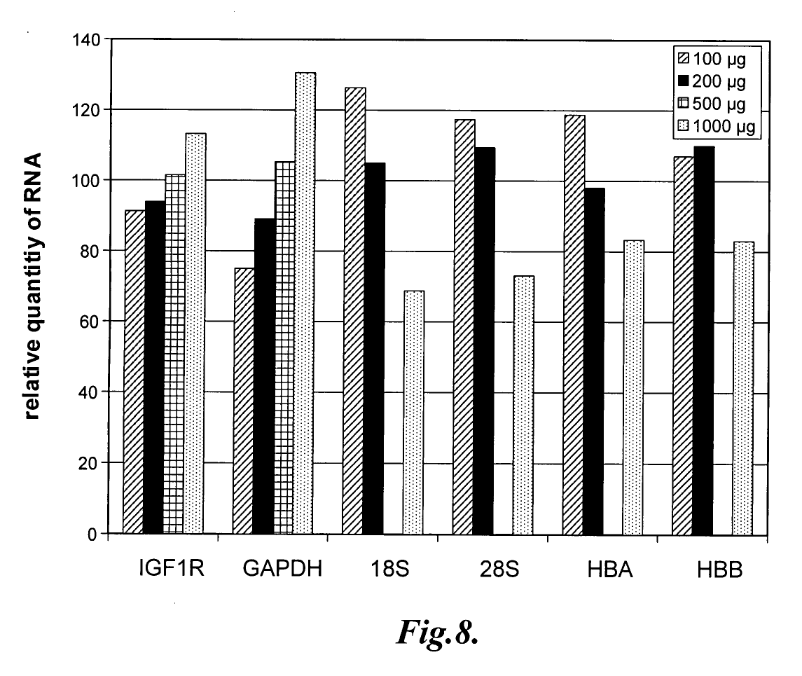

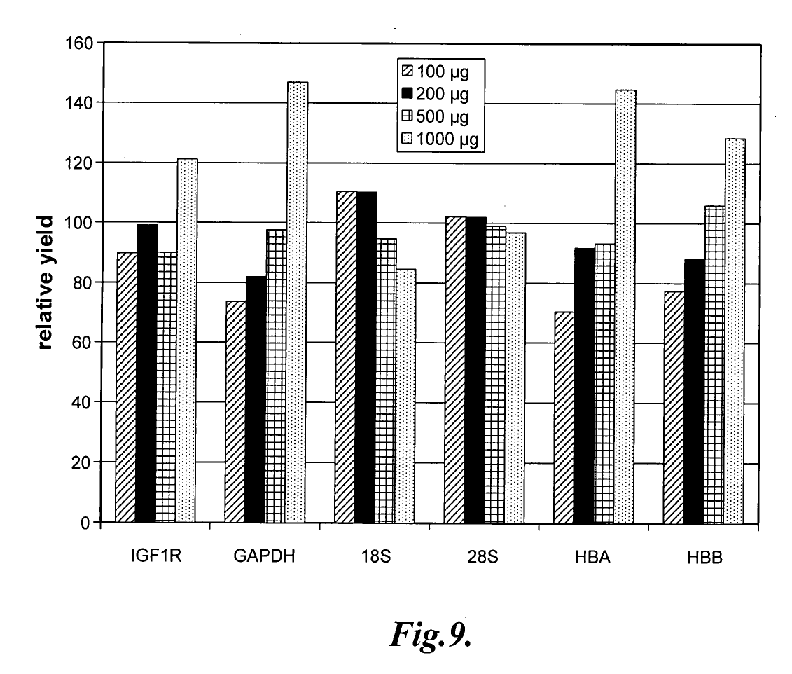

FIG. 7 is a histogram plot on a logarithmic scale showing the relative composition of T7-NSR primed and T7-N7 primed cDNA (normalized of N8) following amplification by in vitro transcription. FIG. 8 is a histogram plot showing the relative abundance of in vitro transcription products from T7-NSR primed cDNA as a function of RNA template amount. FIG. 9 is a histogram plot showing the relative abundance of in vitro transcription products from T7-N7-primed cDNA as a function of RNA template amount.

As shown in FIG. 7 and FIG. 8, for the NSR-primed IVT product, there is a trend toward higher specificity with higher RNA template amounts (increase in IGF1R and GAPDH with a corresponding decrease in rRNA and globin aRNA). As shown in FIG. 9, for the T7-N7 primed IVT product, the IGF1R, GAPDH, and globin levels increased with increasing RNA template, but the rRNAs declined slightly. These results indicate that the NSR-primed IVT product is fairly uniform across a 10-fold range of RNA inputs.

In summary, high dNTP concentrations in combination with the NSR primers result in increased reporter levels and decreased background levels following first strand cDNA synthesis and IVT across a range of RNA template amounts. The composition of the NSR-primed IVT product is substantially different than the T7-N7 and N8 primed product, such that IGF1R and GAPDH levels increased while rRNA and globin levels decreased when primed with NSR, but were not substantially different than the N8-primed starting material when primed with T7-N7. Finally, the composition of the NSR-primed IVT product was relatively uniform across a range of RNA template amounts.

TABLE 31
Reporter to background ratios for NSR and T7-N7 primed first strand cDNA
carried through IVT for different RNA template amounts, normalized to N8.

| | | NSR | | | | T7-N7 | | | |
|---|---|---|---|---|---|---|---|---|---|
| | | 1000 | 500 | 200 | 100 | 1000 | 500 | 200 | 100 |
| IGF1R/18S | 1st strand/N8 | 20 | 15 | 3 | 0 | 2 | 1 | 0 | 0 |
| | IVT/N8 | 75 | 0 | 9 | 1 | 1 | 0 | 0 | 0 |
| 1GF1R/28S | 1st strand/N8 | 7 | 6 | 7 | 6 | 2 | 2 | 2 | 2 |
| | IVT/N8 | 187 | 0 | 148 | 139 | 3 | 3 | 3 | 3 |
| IGF1R/HBA | 1st strand/N8 | 29 | 19 | 14 | 10 | 2 | 1 | 1 | 1 |
| | IVT/N8 | 27 | 0 | 18 | 15 | 2 | 2 | 2 | 2 |
| IGF1R/HBB | 1st strand/N8 | 10 | 10 | 8 | 6 | 1 | 2 | 2 | 2 |
| | IVT/N8 | 69 | 0 | 76 | 85 | 1 | 1 | 2 | 2 |
| GAPDH/18S | 1st strand/N8 | 36 | 23 | 5 | 0 | 3 | 2 | 0 | 0 |
| | IVT/N8 | 41 | 0 | 4 | 0 | 0 | 0 | 0 | 0 |
| GAPDH/28S | 1st strand/N8 | 12 | 10 | 11 | 9 | 3 | 3 | 3 | 2 |
| | IVT/N8 | 104 | 0 | 67 | 55 | 2 | 1 | 1 | 1 |
| GAPDH/HBA | 1st strand/N8 | 50 | 29 | 22 | 15 | 2 | 2 | 1 | 1 |
| | IVT/N8 | 15 | 0 | 8 | 6 | 1 | 1 | 1 | 1 |
| GAPDH/HBB | 1st strand/N8 | 18 | 16 | 12 | 9 | 2 | 2 | 2 | 2 |
| | IVT/N8 | 38 | 0 | 35 | 34 | 0 | 0 | 1 | 1 |

TABLE 32
Ratio of ratios for NSR versus T7-N7 primed first strand cDNA
using the data from Table 24.

NSR/T7-N7

| | | 1000 | 500 | 200 | 100 |
|---|---|---|---|---|---|
| IGF1R/18S | 1st strand | 10 | 10 | 12 | 13 |
| | IVT | 99 | | 85 | 76 |
| IGF1R/28S | 1st strand | 3 | 3 | 3 | 4 |
| | IVT | 62 | | 44 | 44 |
| IGF1R/HBA | 1st strand | 16 | 15 | 13 | 12 |
| | IVT | 17 | | 9 | 6 |
| IGF1R/HBB | 1st strand | 8 | 6 | 5 | 4 |
| | IVT | 85 | | 44 | 43 |
| GAPDH/18S | 1st strand | 13 | 12 | 14 | 14 |
| | IVT | 102 | | 106 | 83 |
| GAPDH/28S | 1st strand | 4 | 4 | 4 | 4 |
| | IVT | 64 | | 55 | 48 |
| GAPDH/HBA | 1st strand | 22 | 18 | 16 | 13 |
| | IVT | 18 | | 12 | 7 |
| GAPDH/HBB | 1st strand | 10 | 8 | 6 | 4 |
| | IVT | 87 | | 55 | 47 |

TABLE 33
qPCR of NSR, T7-N7 and N8 primed cDNA and IVT-RT products
across a range of RNA template amounts. Raw Ct data.

| | | NSR | | | | T7-N7 | | | |
|---|---|---|---|---|---|---|---|---|---|
| | | 1000 | 500 | 200 | 100 | 1000 | 500 | 200 | 100 |
| | 1st strand | 25 | 26 | 27 | 28 | 25 | 26 | 27 | 28 |
| IGF1R | IVT | 21 | 21 | 21 | 21 | 24 | 25 | 25 | 25 |
| | N8 | 24 | 25 | 26 | 27 | 40 | | | |
| | 1st strand | 22 | 23 | 24 | 25 | 22 | 23 | 24 | 25 |
| GAPDH | IVT | 19 | 20 | 20 | 20 | 23 | 23 | 23 | 24 |
| | N8 | 22 | 22 | 23 | 24 | 40 | | | |
| | 1st strand | 24 | 23 | 24 | 25 | 23 | 22 | 23 | 23 |
| ACTIN | IVT | 25 | 25 | 25 | 26 | 25 | 26 | 27 | 27 |
| | N8 | 21 | 21 | 21 | 22 | 40 | | | |
| | 1st strand | 26 | 26 | 27 | 27 | 24 | 24 | 25 | 26 |
| RPO | IVT | 22 | 22 | 23 | 23 | 23 | 23 | 23 | 23 |
| | N8 | 22 | 23 | 23 | 24 | 40 | | | |
| | 1st strand | 19 | 19 | 20 | 21 | 15 | 16 | 16 | 17 |
| 18S | IVT | 17 | 26 | 16 | 16 | 13 | 13 | 13 | 13 |
| | N8 | 14 | 15 | 18 | 22 | 31 | | | |
| | 1st strand | 18 | 18 | 19 | 20 | 16 | 16 | 17 | 18 |
| 28S | IVT | 19 | 27 | 18 | 18 | 16 | 16 | 16 | 16 |
| | N8 | 14 | 15 | 15 | 16 | 34 | | | |
| | 1st strand | 21 | 21 | 21 | 22 | 16 | 17 | 17 | 18 |
| HBA | IVT | 16 | 24 | 16 | 16 | 16 | 16 | 16 | 17 |
| | N8 | 15 | 16 | 17 | 17 | 40 | | | |
| | 1st strand | 20 | 20 | 21 | 21 | 17 | 17 | 18 | 19 |
| HBB | IVT | 19 | 28 | 18 | 18 | 16 | 16 | 16 | 16 |
| | N8 | 16 | 16 | 17 | 17 | 40 | | | |

TABLE 34
Raw Abundance Values

| | | NSR | | | | T7-N7 | | | |
|---|---|---|---|---|---|---|---|---|---|
| | | 1000 | 500 | 200 | 100 | 1000 | 500 | 200 | 100 |
| | 1st strand | 216 | 143 | 72 | 40 | 310 | 183 | 89 | 42 |
| IGF1R | IVT | 4523 | 4053 | 3754 | 3648 | 448 | 333 | 366 | 332 |
| | N8 | 459 | 276 | 149 | 84 | 0 | | | |
| | 1st strand | 2486 | 1423 | 747 | 366 | 2630 | 1505 | 739 | 274 |
| GAPDH | IVT | 16323 | 13161 | 11140 | 9377 | 1570 | 1043 | 876 | 788 |
| | N8 | 2994 | 1780 | 973 | 538 | 0 | | | |
| | 1st strand | 717 | 903 | 711 | 417 | 1681 | 2037 | 1572 | 903 |
| ACTIN | IVT | 394 | 391 | 212 | 205 | 232 | 108 | 95 | 73 |
| | N8 | 3567 | 5356 | 4174 | 2450 | 0 | | | |
| | 1st strand | 175 | 173 | 99 | 74 | 756 | 575 | 291 | 114 |
| RPO | IVT | 2414 | 1749 | 1393 | 1568 | 1484 | 1661 | 1225 | 1481 |
| | N8 | 2488 | 1515 | 891 | 534 | 0 | | | |
| | 1st strand | 17434 | 14200 | 7952 | 4526 | 243626 | 186097 | 113212 | 63707 |
| 18S | IVT | 99195 | | 151603 | 182491 | 969080 | 1084588 | 1263635 | 1266322 |
| | N8 | 754809 | 406684 | 52542 | 3134 | 5 | | | |
| | 1st strand | 34977 | 29249 | 16352 | 10477 | 147574 | 116224 | 70189 | 41750 |
| 28S | IVT | 26417 | | 39545 | 42413 | 162609 | 166203 | 171356 | 171603 |
| | N8 | 501934 | 352785 | 232936 | 135033 | 0 | | | |
| | 1st strand | 5534 | 5188 | 3643 | 2714 | 127644 | 99869 | 58543 | 34664 |
| HBA | IVT | 123460 | | 145322 | 176027 | 208179 | 134168 | 132001 | 101438 |
| | N8 | 335396 | 189786 | 103744 | 58823 | 0 | | | |
| | 1st strand | 7939 | 7090 | 6180 | 4696 | 87383 | 58730 | 38326 | 20360 |
| HBB | IVT | 24468 | | 32431 | 31560 | 205020 | 169267 | 140599 | 123574 |
| | N8 | 171594 | 140251 | 97796 | 61407 | 0 | | | |

### Example 10

This Example describes an exemplary method of using NSR-primed first strand reverse transcription, followed by second strand synthesis and in vitro transcription for the preparation of microarray samples.

Methods:

First Strand Reverse Transcription

First Strand Reverse Transcription is carried out as follows:

Combine:
1 μg total RNA template
2 μl of 100 μM stock NSR primer
x μl of water to a final volume of 10 μl
Mix and incubate at 65° C. for 5 minutes, snap chill on ice.

Add 10 μl of RT cocktail (prepared on ice) containing:
4 μl 5×FS buffer
1.6 μl of 25 mM dNTPs
1.4 μl water
1 μl stock DTT
1 μl RNAs OUT
1 μl SSIII

The sample is mixed, centrifuged and transferred to a 40° C. pre-warmed thermal cycler (to provide a “hot start”), and the sample is incubated at 40° C. for three hours, 70° C. for 10 minutes and chilled to 4° C.

Second Strand Synthesis

A second strand synthesis cocktail is prepared using the components of the SuperScript™ RNA Amplification System (Invitrogen, Catalog No. L1016-01) as follows:
91 μl DEPC-treated water
30 μl 5× Second-strand Buffer
3 μl 10 mM dNTP Mix
4 μl DNA Polymerase I (10 units/μl)
1 μl DNA Ligase (10 units/μl)
1 μl RNAse H (2 units/μl)

130 μl of the second strand synthesis cocktail is added to the 20 μl first strand reaction described above, mixed and the sample is incubated at 16° C. for two hours.

Purification of Double-Stranded cDNA

1. 500 μl of cDNA Loading Buffer is added to the reaction tube from Second-Strand cDNA Synthesis. The total volume in the tube should be 650 μl. Mix thoroughly by pipetting up and down.

2. Each Spin Cartridge is pre-inserted into a collection tube. Load the cDNA/buffer solution directly onto the Spin Cartridge.

3. Centrifuge at 12,000×g at room temperature in a microcentrifuge for 1 minute. Remove the collection tube and discard the flow-through.

4. Place the Spin Cartridge in the same collection tube and add 700 μl of cDNA Wash Buffer.

5. Centrifuge at 12,000×g at room temperature for 2 minutes. Remove the collection tube and discard the flow-through.

6. Place the Spin Cartridge in the same collection tube and centrifuge at 12,000×g at room temperature for an additional 4 minutes. Remove the collection tube and discard the flow-through.

7. Place the Spin Cartridge into a new Recovery Tube.

8. Add 28 μl of DEPC-treated water to the center of the Spin Cartridge and incubate at room temperature for 2 minutes.

9. Centrifuge at 12,000×g at room temperature for 1 minute.

10. Pipette cDNA back onto column, let sit 1 min, and recentrifuge.

In Vitro Transcription Reaction

An In Vitro Transcription Cocktail is prepared as follows (18 μl volume)

| | |
|---|---|
| 1.5 μl | 100 mM ATP |
| 1.5 μl | 100 mM CTP |
| 1.5 μl | 100 mM GTP |
| 0.75 μl | 100 mM UTP |
| 1.75 μl | Amino-allyl UTP (50 mM) |
| 4.0 μl | 10X T7 Reaction Buffer |
| 7.0 μl | T7 Enzyme Mix |

22 μl of purified cDNA is added to the cocktail, and the sample is incubated at 37° C. overnight.

Purification of aRNA

The following procedure is used to purify the aRNA.

1. Add 160 μl of aRNA Binding Buffer to the reaction tube. The total volume should be 200 μl. Mix thoroughly by pipetting up and down.

2. Add 100 μl of 100% ethanol to the reaction tube. Mix thoroughly by pipetting up and down.

3. Each Spin Cartridge is pre-inserted into a collection tube. Load the entire aRNA/buffer solution directly onto the Spin Cartridge.

4. Centrifuge at 12,000×g in a microcentrifuge for 15 seconds at room temperature. Remove the collection tube and discard the flow-through.

5. Place the Spin Cartridge in the same collection tube and add 500 μl of aRNA Wash Buffer.

6. Centrifuge at 12,000×g for 15 seconds at room temperature. Remove the collection tube and discard the flow-through.

7. Repeat Steps 5-6.

8. Place the Spin Cartridge in the same collection tube and centrifuge at full speed for an additional 2 minutes to dry the column. Remove the collection tube and discard the flow-through.

9. Place the Spin Cartridge into a new Recovery Tube.

10. Add 100 μl of DEPC-treated water to the center of the Spin Cartridge and incubate at room temperature for 1 minute.

11. Centrifuge at 12,000×g for 2 minutes at room temperature to collect the purified aRNA in the eluate.

12. Quantify aRNA yield using Nanodrop.

### Example 11

This Example shows that cDNA generated using the 933 6-mers (SEQ ID NOS:1-933 either with or without T7 promoter (SEQ ID NO:934) covalently attached) can be converted into aDNA by PCR.

Methods:

NSR primers used in this Example were as follows:

The 933 6-mers (SEQ ID NOS:1-933) plus a single random nucleotide inserted between the 6-mer sequence and the T7 promoter (SEQ ID NO:934), referred to as “NSR7.”

Total RNA for the cell line Jurkat (T lymphocyte, ATCC No. TIB-152) was obtained from Ambion, Inc. (Austin, Tex.).

The protocol involves a three-step amplification approach. (1) first strand cDNA is generated from RNA using reverse transcription that is primed with NSR primers comprising a first primer binding site to generate NSR primed first strand cDNA, (2) second strand cDNA synthesis that is primed with random primers (e.g., 9-mers) comprising a second primer binding site; and (3) PCR amplification of the cDNA that is primed with forward and reverse primers that bind to the first and second primer binding sites to generate amplified DNA (aDNA).

TABLE 35

Representative Primer Sets for use with NSR
primed first strand reverse transcription
followed by second strand synthesis and PCR
amplification using NSR7 primers including
the T7 promoter (comprising 933 6-mers
(SEQ ID NOS: 1-933) plus a single random
nucleotide inserted between the 6-mer
sequence and the T7 promoter (SEQ ID NO:934))

Primer Set 1

| | | |
|---|---|---|
| T7 primer 1 | CGCAATTAATACGACTCACTATAG | SEQ ID NO:946 |
| | G | |
| R RT 1 | TGCATTGAGAGGGTGTAATTTGNN | SEQ ID NO:947 |
| | NNNNNNN | |
| R PCR 1 | TGCATTGAGAGGGTGTAATTTG | SEQ ID NO:948 |
| Primer Set 2 | | |
| T7 primer 1 | cgcAATTAATACGACTCACTATAG | SEQ ID NO:946 |
| | G | |
| R RT 2 | GTATCTTGGGCCTGTTGACTTCNN | SEQ ID NO:949 |
| | NNNNNNN | |
| R PCR 2 | GTATCTTGGGCCTGTTGACTTC | SEQ ID NO:950 |
| Primer Set 3 | | |
| T7 Primer 1 | cgcAATTAATACGACTCACTATAG | SEQ ID NO:946 |
| | G | |
| R RT 3 | ATTGTTCCTTCAGCTGTTCCATNN | SEQ ID NO:951 |
| | NNNNNNN | |
| R PCR 3 | ATTGTTCCTTCAGCTGTTCCAT | SEQ ID NO:952 |
| Primer Set 4 | | |
| T7 Primer 1 | cgcAATTAATACGACTCACTATAG | SEQ ID NO:946 |
| | G | |
| R RT 4 | CCTCCACACTCCTAAGGTCATCNN | SEQ ID NO:953 |
| | NNNNNNN | |
| R PCR 4 | CCTCCACACTCCTAAGGTCATC | SEQ ID NO:954 |
| Primer Set 5 | | |
| T7 Primer 2 | gcgcAATTAATACGACTCACTA | SEQ ID NO:955 |
| R RT 1 | TGCATTGAGAGGGTGTAATTTGNN | SEQ ID NO:947 |
| | NNNNNNN | |
| R PCR 1 | TGCATTGAGAGGGTGTAATTTG | SEQ ID NO:948 |
| Primer Set 6 | | |
| T7 Primer 2 | gcgcAATTAATACGACTCACTA | SEQ ID NO:955 |
| R RT 2 | GTATCTTGGGCCTGTTGACTTCNN | SEQ ID NO:949 |
| | NNNNNNN | |
| R PCR 2 | GTATCTTGGGCCTGTTGACTTC | SEQ ID NO:950 |
| Primer Set 7 | | |
| T7 primer 2 | gcgcAATTAATACGACTCACTA | SEQ ID NO:955 |
| R RT 3 | ATTGTTCCTTCAGCTGTTCCATNN | SEQ ID NO:951 |
| | NNNNNNN | |
| R PCR 3 | ATTGTTCCTTCAGCTGTTCCAT | SEQ ID NO:952 |
| Primer Set 8 | | |
| T7 Primer 2 | gcgcAATTAATACGACTCACTA | SEQ ID NO:955 |
| R RT 4 | CCTCCACACTCCTAAGGTCATCNN | SEQ ID NO:953 |
| | NNNNNNN | |
| R PCR 4 | CCTCCACACTCCTAAGGTCATC | SEQ ID NO:954 |

Note: the protocol described below was carried out with all the primers shown above in TABLE 35, however it is briefly described with reference to Primer Set 1 in order to clearly present the three method steps:

First Strand cDNA synthesis: was primed with the NSR7 primer pool comprising the T7 promoter (SEQ ID NO:934) plus one random nucleotide (N) plus the 933 6-mers (SEQ ID NOS:1-933)

Second Strand cDNA synthesis: was carried out using the following primer pool:

5′ TGCATTGAGAGGGTGTAATTTGNNNNN 3′ (SEQ ID NO:947) (referred to as R RT 1 comprising random 9-mers plus second PCR primer binding site, or “tail”).

PCR Amplification was primed using the following PCR Primers:

Forward PCR 1: 5′ CGCAATTAATACGACTCACTATAGG 3′ (SEQ ID NO:946) (binds to T7 promoter (first primer binding site))

Reverse PCR 1: 5′ TGCATTGAGAGGGTGTAATTTG 3′ (SEQ ID NO:947)

(binds to second PCR primer binding site)

Additional Primer Sets Useful for Generating aDNA:

Although the primer sets described above in Table 35 are used with NSR7 including the T7 promoter, it will be understood that the method of producing aDNA may be practiced using a pool of not-so-random primers (NSR) tailed with any defined sequence containing at least one primer binding sequence. For example, a primer pool was used comprising a pool of NSR primers that included the 933 6-mers (SEQ ID NOS:1-933) plus a single random nucleotide inserted between the 6-mer sequence and the first primer binding sequence, SEQ ID NO:956: 5′ CCGAACTACCCACTTGCATT 3′

First strand synthesis was carried out using the NSR primer pool comprising the first primer binding sequence (SEQ ID NO:956) plus one random nucleotide (N) plus the 933 6-mers (SEQ ID NOS:1-933).

Second strand synthesis was carried out using the following primer pool:

5′ CCACTCCATTTGTTCGTGTGNNNNNN 3′ (SEQ ID NO:957) (comprising random 9-mers plus a second PCR primer binding site.)

PCR amplification of the double-stranded cDNA was then carried out using the following PCR primers:

Forward PCR Primer: 5′ CCGAACTACCCACTTGCATT 3′ (SEQ ID NO:958) (binds to first PCR primer binding site)

Reverse PCR Primer: 5′ CCACTCCATTTGTTCGTGTG 3′ (SEQ ID NO:959) (binds to second PCR primer binding site)

Detailed Methods

Test Samples: Test samples 1-8 were carried out using Primer sets 1-8 shown above in TABLE 35 and were tested in parallel as described for primer set #1 below.

Control samples were included as follows:

1. A first strand reverse transcriptase reaction into second strand Klenow, but no N9 primer, followed by PCR amplification.

2. A first strand reverse transcriptase reaction, no second strand Klenow, followed by PCR amplification.

First Strand Reverse Transcription:

First strand reverse transcription was carried out as follows:

Combine:
1 μl of 1 μg/μl Jurkat total RNA template (obtained from Ambion, Inc. (Austin, Tex.)).
2 μl of 100 μM stock NSR7 primer pool
7 μl H₂O to a final volume of 10 μl.

Mixed and incubated at 65° C. for 5 minutes, snap chilled on ice.

Added 10 μl of RT cocktail (prepared on ice) containing:
4 ul 5× First Strand Buffer (250 mM Tris-HCL, pH 8.3, 375 mM KCl, 15 mM MgCl₂)
2 μl 20 mM dNTP
1 μl H₂O
1 μl 0.1 M DTT
1 μl RNAse OUT (Invitrogen)
1 μl MMLV reverse transcriptase (200 units/μl) (SuperScript III™ (SSIII), Invitrogen Corporation, Carlsbad, Calif.)

The sample was mixed and transferred to a 40° C. pre-warmed thermal cycler (to provide a “hot start”), and the sample was then incubated at 40° C. for two hours, 70° C. for 10 minutes and chilled to 4° C.

1 μl of RNAse H (1-4 units/μl) was then added and the sample was incubated at 37° C. for 20 minutes, then heated to 95° C. for 5 minutes and snap chilled at 4° C.

Second Strand Synthesis

A second strand synthesis cocktail was prepared as follows:
10 μl 10× Klenow Buffer
10 μl N9 Reverse RT 1 Primer (SEQ ID NO:947)
2.5 μl 20 mM dNTPs
57.5 μl H₂O
0.33 μl Klenow enzyme (50 U/μl of NEB exo-)

80 μl of the second strand synthesis cocktail was added to the 20 μl first strand template reaction mixture, mixed and incubated at 37° C. for 30 minutes.

cDNA Purification

The resulting double stranded cDNA was purified using Spin Cartridges obtained from Invitrogen and buffers supplied in the kit according to the manufacturer's directions. A total volume of 30 μl was eluted from the column, of which 20 μl was used for follow-on PCR.

PCR Amplification

The following mixture was added to the 20 μl of purified cDNA template:
20 μl 5× Roche Expand Plus PCR Buffer
2.5 μl 20 mM dNTPS
4 μl Forward PCR Primer (10 mM stock) (SEQ ID NO:946)
4 μl Reverse PCR Primer (10 mM stock) (SEQ ID NO:947)
1 μl Expand Enzyme (5 U/μl) (Roche)
40 μl H₂O
10 μl 25 mM MgCl₂

PCR Amplification Conditions:

25 total PCR cycles:

10 cycles of:
94° C. for 30 seconds
55° C. or 60° C. for 30 seconds
72° C. for 1 minute

15 cycles of:
94° C. for 30 seconds
55° C. or 60° C. for 30 seconds
72° C. for 1 minute (plus 10 seconds added to the elongation step with each cycle)
72° for 7 minutes to polish and chilled at 4° C.

Results: The results were analyzed in terms of (1) measuring the level of amplification of selected reporter genes by qPCR, (2) measuring amplified DNA “aDNA” yield, and (3) evaluation of an aliquot of the aDNA on an agarose gel to confirm that the population of species in the cDNA were equally represented.

The data in TABLE 36 shows the raw abundance values (adjusted for dilution) for reporter genes GAPDH, GUSB, hPO and Actin following qPCR of the double-stranded cDNA and aDNA products for samples 1-10.

TABLE 36
qPCR Values (raw values adjusted for dilution)

| | | 1 | 2 | 3 | 4 | 5 | 6 | 7 | 8 | 9 | 10 |
|---|---|---|---|---|---|---|---|---|---|---|---|
| GAPDH | ds-cDNA | 7380 | 6881 | 6122 | 9532 | 8173 | 7594 | 839 | 3585 | 7570 | 323 |
| | PCR-aDNA | 11990 | 19366 | 25184 | 35564 | 9926 | 17131 | 11701 | 15143 | 10564 | 34 |
| GUSB | ds-cDNA | 20 | 20 | 18 | 25 | 16 | 23 | 2 | 14 | 20 | 1 |
| | PCR-aDNA | 430 | 898 | 262 | 507 | 368 | 938 | 117 | 275 | 500 | 3 |
| hPO | ds-cDNA | 2365 | 2300 | 1578 | 2095 | 2490 | 2214 | 207 | 795 | 1097 | 54 |
| | PCR-aDNA | 32525 | 34686 | 14460 | 25778 | 20979 | 36267 | 5086 | 17396 | 4685 | 17 |
| Actin | ds-cDNA | 3088 | 2553 | 2362 | 3412 | 2829 | 3385 | 171 | 1417 | 2702 | 254 |
| | PCR-aDNA | 16551 | 11425 | 7172 | 9476 | 14503 | 13920 | 3305 | 4479 | 36772 | 174 |

The raw data shown above in Table 36 was then adjusted based on the following amplification factor:

(PCR/cDNA)*(cDNA/average cDNA)

This amplification factor was introduced to weight amplification in favor of samples that had robust cDNA synthesis levels and robust PCR yields (e.g., sample 2), over samples with poor cDNA and PCR yields, but high levels of cDNA to PCR amplification (e.g., sample 7). The abundance values, adjusted based on the amplification factor described above, are shown below in TABLE 37.

TABLE 37
qPCR results (amplification factor)

| | | | | | | | | | Control | Control |
|---|---|---|---|---|---|---|---|---|---|---|
| | Sample | | | | | | | | #1: No | #2: No |
| | 1 | 2 | 3 | 4 | 5 | 6 | 7 | 8 | N9 Primer | Klenow step |
| μg PCR | 4.1 | 4.2 | 3.1 | 3.3 | 2.5 | 3.4 | 2.3 | 2.1 | 3.3 | 0.4 |
| yield | | | | | | | | | | |
| GAPDH | 2.1 | 3.3 | 4.3 | 6.1 | 1.7 | 3.0 | 2.0 | 2.6 | 1.8 | 0.0 |
| GUSB | 27.0 | 56.3 | 16.4 | 31.8 | 23.1 | 58.8 | 7.3 | 17.3 | 31.3 | 0.2 |
| hPO | 21.4 | 22.8 | 9.5 | 17.0 | 13.8 | 23.9 | 3.3 | 11.4 | 3.1 | 0.0 |
| Actin | 7.5 | 5.2 | 3.2 | 4.3 | 6.5 | 6.3 | 1.5 | 2.0 | 16.6 | 0.1 |

As shown above in TABLE 37, PCR yield was generally good, especially with Primer Sets 1 and 2. Amplification was observed for all reporter genes tested. An aliquot of the aDNA from samples 1-8 and controls 1-2 were run on 1.6% agarose gels (not shown) and each test sample showed a smear of amplified material, indicating that the amplified products were representative of mRNA species present in the starting total RNA.

A second set of reactions were run with the same samples using the methods described above, but with the PCR annealing temperature raised to 60° C. with a total of 30 cycles of PCR. qPCR was performed on the resulting aDNA using qPCR for the eight reporter genes shown in TABLE 38 that are known to be highly expressed in Jurkat cells.

TABLE 38
Reporter Genes for Jurkat Cells

| | Gene | |
|---|---|---|
| Assay # | Symbol | Gene Name |
| 1 | STMN1 | stathmin 1/oncoprotein 18 |
| 2 | CDCA7 | cell division cycle associated 7 |
| 3 | PPIA | peptidylprolyl isomerase A (cyclophilin A) |
| 4 | EIF3S3 | eukaryotic translation initiation factor 3, subunit |
| | | 3 gamma, 40 kDa |
| 5 | NUCB2 | nucleobindin 2 |
| 6 | SRP14 | signal recognition particle 14 kDa (homologous |
| | | Alu RNA binding protein) |
| 7 | MAPRE2 | microtubule-associated protein, RP/EB family |
| | | member 2 |
| 8 | NDUFV2 | NADH dehydrogenase (ubiquinone) flavoprotein 2, |
| | | 24 kDa |
| 9 | GAPDH | |
| 10 | GUSB | |
| 11 | hPO | |
| 12 | Actin | |

As shown in TABLE 39, the overall yield of aDNA obtained using an annealing temperature at 60° C. during PCR amplification is greater than that obtained with 55° C. for seven out of the eight primer sets tested.

TABLE 39
PCR yield measured by Nanodrop

| | PCR Primer Set | | | | | | | |
|---|---|---|---|---|---|---|---|---|
| | 1 | 2 | 3 | 4 | 5 | 6 | 7 | 8 |
| 55° C. | 3.1 | 2.6 | 1.6 | 2.8 | 1.1 | 2.6 | 2.1 | 2.1 |
| 60° C. | 3.8 | 3.2 | 2.1 | 3.2 | 1.7 | 2.7 | 2.0 | 2.5 |
| total PCR yield (μg) | 6.9 | 5.8 | 3.7 | 6.0 | 2.9 | 5.2 | 4.1 | 4.7 |

TABLE 40
PCR Specificity as measured by qPCR,
expressed as the ratio of aDNA to cDNA

| Gene | | Primer | | | | | | | |
|---|---|---|---|---|---|---|---|---|---|
| ID | temp | set 1 | 2 | 3 | 4 | 5 | 6 | 7 | 8 |
| 1 | 55 | 97 | 25 | 15 | 21 | 11 | 25 | 9 | 43 |
| 1 | 60 | 188 | 49 | 66 | 62 | 28 | 40 | 18 | 97 |
| 2 | 55 | 9 | 6 | 3 | 3 | 3 | 5 | 2 | 2 |
| 2 | 60 | 17 | 9 | 5 | 4 | 7 | 7 | 3 | 2 |
| 3 | 55 | 101 | 55 | 31 | 68 | 13 | 51 | 23 | 44 |
| 3 | 60 | 105 | 68 | 32 | 89 | 18 | 49 | 34 | 53 |
| 4 | 55 | 38 | 18 | 8 | 18 | 8 | 15 | 7 | 11 |
| 4 | 60 | 38 | 19 | 9 | 26 | 13 | 14 | 10 | 13 |
| 5 | 55 | 35 | 20 | 4 | 5 | 4 | 15 | 3 | 9 |
| 5 | 60 | 75 | 48 | 24 | 18 | 11 | 27 | 6 | 25 |
| 6 | 55 | 10 | 5 | 1 | 1 | 1 | 5 | 2 | 2 |
| 6 | 60 | 21 | 12 | 7 | 3 | 2 | 4 | 3 | 2 |
| 7 | 55 | 39 | 20 | 7 | 14 | 9 | 18 | 5 | 9 |
| 7 | 60 | 53 | 36 | 7 | 20 | 24 | 23 | 6 | 10 |
| 8 | 55 | 61 | 46 | 13 | 28 | 13 | 42 | 8 | 12 |
| 8 | 60 | 67 | 59 | 12 | 37 | 17 | 39 | 12 | 13 |
| 9 | 55 | 1 | 1 | 1 | 2 | 0 | 2 | 1 | 4 |
| 9 | 60 | 3 | 2 | 5 | 5 | 0 | 1 | 1 | 4 |
| 10 | 55 | 16 | 11 | 3 | 4 | 2 | 16 | 3 | 10 |
| 10 | 60 | 20 | 26 | 13 | 17 | 12 | 23 | 6 | 19 |
| 11 | 55 | 10 | 12 | 3 | 9 | 1 | 9 | 3 | 6 |
| 11 | 60 | 10 | 15 | 4 | 11 | 1 | 6 | 3 | 5 |
| 12 | 55 | 4 | 3 | 2 | 2 | 1 | 3 | 3 | 2 |
| 12 | 60 | 3 | 5 | 3 | 3 | 0 | 2 | 2 | 1 |

As shown in TABLE 40, quantitative PCR (qPCR) analysis of the aDNA from PCR reactions annealed at 55° C. or 60° C. showed that primer pair 1 at a 60° C. annealing temperature provided the best PCR specificity as measured by the ratio of aDNA to cDNA. It was further determined that the specific activity of reporter genes tracked closely with aDNA yields over the range of PCR cycles tested (4 cycles to 24 cycles) (data not shown).

### Example 12

This Example demonstrates that aDNA amplified from double stranded cDNA templates generated using the 933 6-mers (SEQ ID NOS:1-933), as described in EXAMPLE 11, preserved the expression ratios observed in the double-stranded cDNA templates.

Methods:

First strand cDNA synthesis was performed as described in Example 11 using 1 μg of total RNA template from Jurkat or K562 cells (obtained from Ambion, Inc. Austin Tex.) with the NSR7 primer pool. Two Jurkat samples were run in parallel. For second strand synthesis and cDNA purification were also carried out as described in Example 11.

PCR Amplification of cDNA

The cDNA template was diluted 10-fold and 100-fold prior to use in PCR reaction, and either 10 μl purified, or 10 μl diluted cDNA was used.

Add to the 10 μl of cDNA template:
20 μl 5× Roche Expand Plus PCR Buffer
2.0 μl 10 mM dNTPS
4 μl Forward PCR Primer (10 mM stock) (SEQ ID NO:946)
4 μl Reverse PCR Primer (10 mM stock) (SEQ ID NO:947)
1 μl Expand Enzyme (5 U/μl) (Roche)
60 μl H₂O
10 μl 25 mM MgCl₂

PCR Amplification Conditions:

Perform 30 total PCR cycles:

10 cycles of:
94° C. for 30 seconds
60° C. for 30 seconds
72° C. for 1 minute

20 cycles of:
94° C. for 30 seconds
60° C. for 30 seconds
72° C. for 1 minute (plus 10 seconds added to the elongation step with each cycle)
72° C. for 7 minutes to polish and chill at 4° C.

Post-PCR clean-up: 100 μl PCR reaction was diluted with 500 μl buffer and purified using Spin Cartridges obtained from Invitrogen and buffers supplied in the kit according to the manufacturer's directions. A total volume of 55 μl was eluted from the column.

Results: The results were analyzed in terms of total aDNA yield, evaluation of aDNA on agarose gels to verify the presence of the expected smear indicating amplification of substantially all species in the transcriptome, and by measuring a panel of reporter genes by qPCR.

TABLE 41
Yield of PCR product (aDNA) measured by Nanodrop

| | ug total aDNA in PCR |
|---|---|
| PCR Sample | Reaction (nanodrop) |
| Jurkat #1 (10 μl) | 2.6 |
| Jurkat #2 (10 μl) | 2.5 |
| K562 #1 (10 μl) | 1.7 |
| Jurkat #1 (1 μl) | 2.2 |
| Jurkat #2 (1 μl) | 2.1 |
| K562 #1 (1 μl) | 1.9 |
| Jurkat #1 (0.1 μl) | 2.0 |
| Jurkat #2 (0.1 μl) | 1.9 |
| K562 #1 (0.1 μl) | 2.0 |
| no template control | 0.5 |

Results: As shown in TABLE 41, the yields of aDNA were consistent across the range of template inputs. These results indicate that the PCR reaction was saturated at the lower template input. The agarose gel analysis showed the expected smear indicating good amplification across the transcriptome (not shown).

In order to determine if the aDNA generated from the cDNA preserved the target genes expression levels present in the cDNA, quantitative PCR analysis was conducted with 54 randomly chosen TaqMan reagents, detecting Gene IDS #1-34. As shown in TABLE 42, of the 54 randomly chosen target genes, measurable signal was measured for 34 genes in both NSR7-primed cDNA and aDNA generated therefrom (from 10 μl cDNA template input). The gene expression signal shown below in Table 42 was calculated as power (10 (log 10(½)*Ct+10)).

TABLE 42
qPCR abundance values. Values expressed as 10 raised to the
(Cts × log10(½) + 10) power

| | | | | aDNA (from 10 ul | | |
|---|---|---|---|---|---|---|
| | cDNA | | | input Template) | | |
| | Jurkat | Jurkat | | Jurkat | Jurkat | |
| Gene ID | #1 | #2 | K562 #1 | #1 | #2 | K562 #1 |
| 1 | 10 | 7 | 5 | 57 | 50 | 98 |
| 2 | 12 | 14 | 8 | 87 | 109 | 8 |
| 3 | 15 | 54 | 0 | 339 | 619 | 24 |
| 4 | 20 | 28 | 47 | 245 | 277 | 500 |
| 5 | 447 | 954 | 160 | 16840 | 27201 | 7321 |
| 6 | 1726 | 5528 | 1458 | 349878 | 455053 | 202163 |
| 7 | 0 | 1 | 0 | 3 | 30 | 9 |
| 8 | 93 | 273 | 17 | 7315 | 10373 | 1010 |
| 9 | 166 | 417 | 594 | 11335 | 11122 | 16157 |
| 10 | 255 | 522 | 238 | 17105 | 19957 | 8002 |
| 11 | 1 | 3 | 0 | 79 | 107 | 5 |
| 12 | 7130 | 11637 | 1062 | 635546 | 790841 | 177300 |
| 13 | 281 | 642 | 76 | 11218 | 13634 | 2909 |
| 14 | 364 | 1141 | 83 | 21810 | 26586 | 3022 |
| 15 | 40 | 97 | 0 | 3499 | 3570 | 20 |
| 16 | 1 | 1 | 0 | 4 | 10 | 1 |
| 17 | 24 | 68 | 24 | 1153 | 1366 | 915 |
| 18 | 2764 | 7517 | 598 | 59021 | 62854 | 14790 |
| 19 | 688 | 1362 | 288 | 24491 | 33524 | 13175 |
| 20 | 65 | 148 | 23 | 3900 | 3834 | 655 |
| 21 | 8 | 19 | 4 | 138 | 189 | 52 |
| 22 | 600 | 1011 | 205 | 118107 | 129372 | 54354 |
| 23 | 0 | 2 | 0 | 14 | 45 | 5 |
| 24 | 1059 | 3831 | 87 | 117411 | 123053 | 4357 |
| 26 | 15 | 33 | 4 | 468 | 673 | 12 |
| 27 | 27 | 94 | 68 | 5051 | 5570 | 5316 |
| 29 | 184 | 236 | 0 | 932 | 1105 | 5 |
| 30 | 29 | 58 | 28 | 3259 | 3095 | 1580 |
| 31 | 201 | 816 | 64 | 5142 | 5825 | 1826 |
| 32 | 2 | 5 | 2 | 72 | 49 | 103 |
| 33 | 105 | 258 | 82 | 1978 | 2770 | 2260 |
| 34 | 836 | 1560 | 513 | 3312 | 3906 | 2202 |

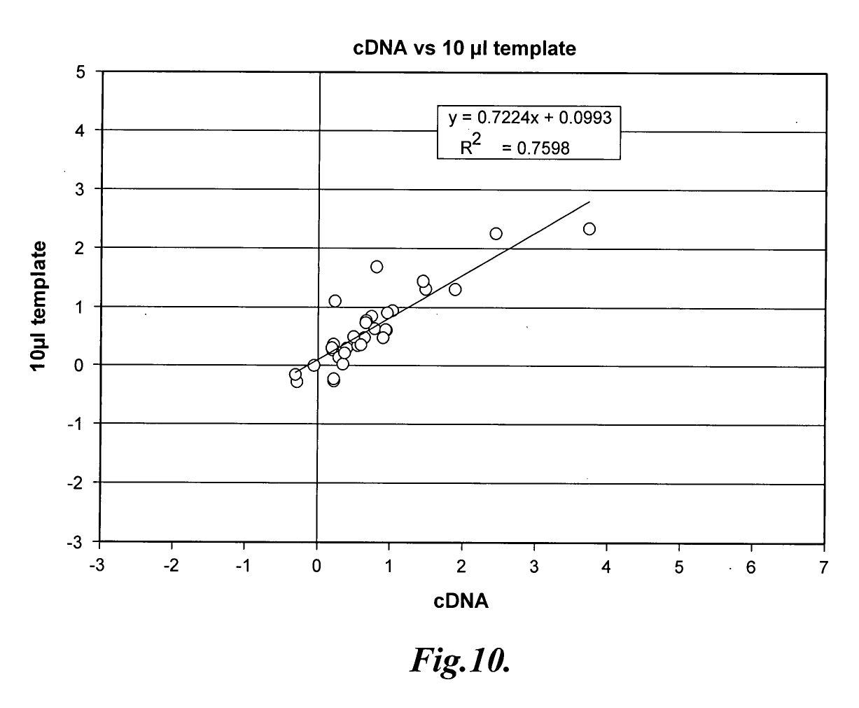

As shown in TABLE 42, the results of the Jurkat and K562 experiments show a good correlation in expression ratios between the cDNA and the aDNA derived from the cDNA. FIG. 10 shows a plot of the expression values from TABLE 42 measured in the cDNA (x-axis) versus the aDNA (y-axis). Importantly, the results shown in FIG. 10 demonstrate the preservation of expression ratios from the actual sample (cDNA) to the PCR amplified material (aDNA), such that gene x is y-fold amplified by the NSR-PCR regardless of whether its expression is high or low. Overall, these experiments indicates that NSR PCR aDNA may be used to amplify total RNA to aDNA while preserving expression ratios.

### Example 13

This Example demonstrates that aDNA amplified from double stranded cDNA templates generated using the 933 6-mers (SEQ ID NOS:1-933), as described in EXAMPLE 11, preserved the enrichment of target genes relative to rRNA and globin.

Rationale: To determine whether the enrichment of target genes relative to rRNA and globin previously observed from in-vitro transcription products generated from NSR-primer cDNA is preserved in amplified DNA product generated from PCR amplification of the NSR-primed cDNA.

Methods: In order to determine whether the enrichment of target genes relative to rRNA and globin previously observed from in-vitro transcription products generated from NSR-primer cDNA is preserved in amplified DNA product generated from PCR amplification of the NSR-primed cDNA, the amplified aDNA product obtained from cDNA generated using NSR7 primers was compared to the amplified aDNA product obtained from cDNA generated using random 8-mers in the following experiment.

First strand cDNA synthesis was performed on 1 μg of total RNA isolated from whole blood, as described in Example 11 with duplicate samples generated with either (1) the NSR7 primer pool (comprising SEQ ID NOS:1-933 that each had the T7 promoter (SEQ ID NO: 934) covalently attached at the 5′ end); or (2) a random 8-mer (N8) primer pool with the T7 promoter (SEQ ID NO: 934) covalently attached at the 5′ end.

Second strand synthesis and cDNA purification was performed as described in Example 11, with either SEQ ID NO: 947 for both the NSR7 primed samples, and the random 8-mers (N8) primed samples.

PCR Amplification of cDNA

Either 1 μl+9 μl H20 or 10 μl cDNA template was used in the follow-on PCR reaction as follows:

Add to the 10 μl of cDNA template:
20 μl 5× Roche Expand Plus PCR Buffer (includes 1.5 mM MgCl₂)
1.0 μl 20 mM dNTPS
4 μl Forward PCR Primer (10 mM stock) (SEQ ID NO:949)
4 μl Reverse PCR Primer (10 mM stock) (SEQ ID NO:948)
1 μl Expand Enzyme (5 U/μl) (Roche)
60 μl H₂O

PCR Amplification Conditions:

Perform 20 total PCR cycles:

10 cycles of:
94° C. for 30 seconds
60° C. for 30 seconds
72° C. for 1 minute

10 cycles of:
94° C. for 30 seconds
60° C. for 30 seconds
72° C. for 1 minute (plus 10 seconds added to the elongation step with each cycle)
72° for 7 minutes to polish and chill at 4° C.

Post-PCR clean-up: 100 μl PCR reaction was diluted with 500 μl buffer and purified using Spin Cartridges obtained from Invitrogen and buffers supplied in the kit according to the manufacturer's directions. A total volume of 50 μl was eluted from the column.

Results: The data in TABLE 43 shows the ratio of qPCR abundance values for NSR-primed to N8-primed cDNA (adjusted for dilution) for the following reporter genes: A=IGF1R; B=GAPDH, C═STMN1, and D=CDCA7 compared to background gene expression measured for 18S, 28S, HBA1, HBA2 or HBB.

TABLE 43
qPCR results

| | | cDNA #1 | cDNA #2 | PCR #1 | PCR #1 | PCR #2 | PCR #2 |
|---|---|---|---|---|---|---|---|
| | | (5 ul) | (5 ul) | (10 ul) | (1 ul) | (10 ul) | (1 ul) |
| Gene A/18S | NSR/N8 | 79 | 88 | 1162 | 3732 | 1345 | 4677 |
| Gene B/18S | NSR/N8 | 162 | 178 | 103 | 311 | 112 | 310 |
| Gene C/18S | NSR/N8 | 64 | 71 | 732 | 1861 | 846 | 3329 |
| Gene D/18S | NSR/N8 | 87 | 102 | 236 | 937 | 253 | 811 |
| Gene A/28S | NSR/N8 | 23 | 31 | 193 | 300 | 190 | 287 |
| Gene B/28S | NSR/N8 | 46 | 63 | 17 | 25 | 16 | 19 |
| Gene C/28S | NSR/N8 | 18 | 25 | 122 | 150 | 119 | 204 |
| Gene D/28S | NSR/N8 | 25 | 36 | 39 | 75 | 36 | 50 |
| Gene A/HBA | NSR/N8 | 42 | 54 | 159 | 746 | 269 | 532 |
| Gene B/HBA | NSR/N8 | 86 | 111 | 14 | 62 | 22 | 35 |
| Gene C/HBA | NSR/N8 | 34 | 44 | 100 | 372 | 169 | 379 |
| Gene D/HBA | NSR/N8 | 46 | 63 | 32 | 187 | 51 | 92 |
| Gene A/HBB | NSR/N8 | 6 | 7 | 125 | 139 | 155 | 163 |
| Gene B/HBB | NSR/N8 | 13 | 14 | 11 | 12 | 13 | 11 |
| Gene C/HBB | NSR/N8 | 5 | 5 | 79 | 70 | 98 | 116 |
| Gene D/HBB | NSR/N8 | 7 | 8 | 25 | 35 | 29 | 28 |

The data shown above in TABLE 43 shows a dramatic enrichment of target genes over background expression in aDNA amplified from NSR7 primed cDNA. The observed enrichment is consistently better with lower template inputs. Overall, it is clear that the three step amplification method (NSR-PCR) described in EXAMPLE 11 provides a significant enrichment of target genes over rRNAs and globins. Therefore, the NSR-PCR method described in EXAMPLE 11 could be used for full transcriptome profiling of total RNA in a biological sample, such as whole blood.

### Example 14

This Example shows that the yield of amplified DNA can be increased in a PCR reaction through the use of an elevated concentration of primers, dNTPs and MgCl₂, thereby allowing for the implementation of NSR-PCR in high-throughput applications.

Rationale: An Experiment was carried out to measure total aDNA yields from a PCR reaction using a cDNA template as a function of primer input concentrations at two different dNTP and Mg++ concentrations as follows.

Methods:

Template: cDNA was prepared from Jurkat total RNA using NSR7 primers, as described in Example 13.

PCR Primers:

Forward: SEQ ID NO:949; Reverse: SEQ ID NO:948

Primer Concentrations tested: 200, 400, 600, 800, and 1000 nM

dNTP concentrations tested: 200 μM, 1.5 mM

Mg++ concentrations tested: 500 μM, 4 mM

PCR reactions were prepared as described in Example 13.

PCR Amplification Conditions:

Perform 30 total PCR cycles:

10 cycles of:
94° C. for 30 seconds
60° C. for 30 seconds
72° C. for 1 minute

and

20 cycles of:
94° C. for 30 seconds
60° C. for 30 seconds
72° C. for 1 minute (plus 10 seconds added to the elongation step with each cycle)
72° C. for 7 minutes to polish and chill at 4° C.

Post-PCR clean-up: 100 μl PCR reaction was diluted with 500 μl buffer and purified using Spin Cartridges obtained from Invitrogen and buffers supplied in the kit according to the manufacturer's directions. A total volume of 50 μl was eluted from the column.

TABLE 44
Yield of PCR product (aDNA), raw data

| | | | |
|---|---|---|---|
| 200 uM | 500 uM | 200 uM | 500 uM |
| dNTP/ | dNTP/ | dNTP/ | dNTP/ |
| 1.5 mM | 4 mM | 1.5 mM | 4 mM |
| Mg++ | Mg++ | Mg++ | Mg++ |

| | |
|---|---|
| ng PCR | ng PCR |
| product/ | product/100 μl |
| μl cDNA template | PCR reaction |

| | | | | |
|---|---|---|---|---|
| 200 nM dNTPs | 27.96 | 36.47 | 2.796 | 3.647 |
| 400 nM dNTPs | 48.36 | 60.51 | 4.836 | 6.051 |
| 600 nM dNTPs | 54.72 | 76.72 | 5.472 | 7.672 |
| 800 nM dNTPs | 60.61 | 94.32 | 6.061 | 9.432 |
| 1000 nM dNTPs | 59.62 | 96.87 | 5.962 | 9.687 |

The results shown above in TABLE 44 indicate that the yields of aDNA at 800 nM primer, 500 uM dNTPs and 4 mM Mg++ approach 10 μg per a 100 μl PCR reaction. The aDNA was also analyzed by agarose gel and was confirmed to contain a smear of amplified material, indicating that the amplified products were representative of mRNA species present in the starting total RNA.

In order to further assess the presence of amplified full transcript material versus the amplification of non-specific material, the panel of genes 1-8 shown in TABLE 38 were analyzed by qPCR. Control sample 9 was 18S rRNA and control sample 10 was 28S rRNA?

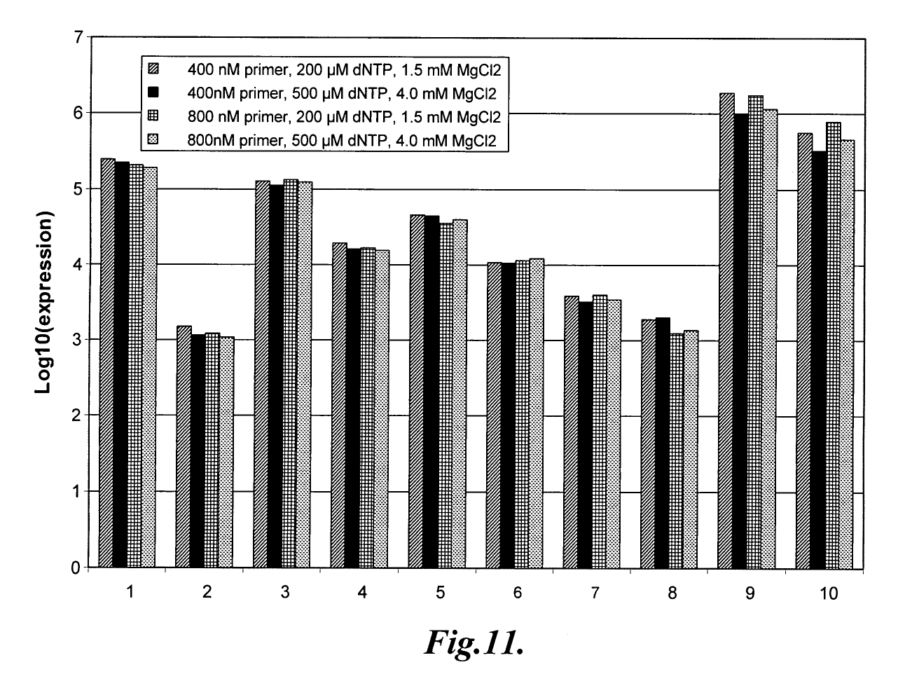

FIG. 11 shows the results of the qPCR plotted as the log 10 expression of each of the genes. As shown, the amplified aDNA yields produced in the PCR reaction having 800 nM primer, 500 uM dNTP and 4 mM MgCl₂ have the same gene specific activities as aDNA produced at lower primer, dNTP and Mg++ concentrations. Although the values of control sample 9 (18S rRNA) and sample 10 (28S rRNA) shown in FIG. 11 are higher than some of the reporter genes, it is important to note that these values (18S and 28S RNA) have actually been reduced by approximately 99 fold in comparison to the amount of rRNA present in samples that are randomly primed (e.g., not primed using the NSR primers). Because rRNA constitutes about 98% of total RNA, even when the abundance of the rRNA is reduced to 1% of the original value, the total amount present still represents about ⅓ of the RNA in the sample. Importantly, this 99% decrease in rRNA observed in samples treated in accordance with the methods of the invention has been observed by the present inventors to be more than sufficient to eliminate the problem of rRNA obscured signal (i.e. “cross-talk”) typically observed in samples that are randomly primed (data not shown).

These data demonstrate that the high yield of aDNA generated using the optimized PCR conditions contain amplified full transcript material, and the increased yield is not due to the presence of non-specific material generated during the amplification reaction.

### Example 15

This Example describes methods that are useful to label the aDNA PCR products for subsequent use in gene expression monitoring applications.

1. Direct Chemical Coupling of Fluorescent Label to the PCR Product

Cy3 and Cy5 direct label kits were obtained from Mirus (Madison, Wis., kit MIR product numbers 3625 and 3725).

10 μg of PCR product (aDNA), obtained as described in Example 11, was incubated with labeling reagent as described by the manufacturer. The labeling reagents covalently attached Cy3 or Cy5 to the nucleic acid sample, which can then be used in almost any molecular biology application, such as gene expression monitoring. The labeled aDNA was then purified and its fluorescence was measured relative to the starting label.

Results: Four aDNA samples were labeled as described above, and fluorescence was measured. A range of 0.9 to 1.5% of retained label was observed across the four labeled aDNA samples (otherwise referred to as a labeling efficiency of 0.9 to 1.5%). These results fall within the 1% to 3% labeling efficiency typically observed for aaUTP labeled, in vitro translated, amplified RNA.

2. Incorporation of aminoallyl modified dUTP (aadUTP) during PCR with an aDNA template using one primer (forward or reverse) to yield aa-labeled, single-stranded aDNA.

Methods: 1 μg of the aDNA PCR product, generated using the NSR7 primer pool as described in Example 11, is added to a PCR reaction mix as follows:
100 to 1000 μM aadUTP+dCTP+cATP+dGTP+dUTP (the optimal balance of aadUTP to dUTP may be empirically determined using routine experimentation)
4 mM MgCl₂
400-1000 nM of only the forward or reverse primer (e.g., Forward: SEQ ID NO:949; or Reverse: SEQ ID NO:948), but not both.

PCR Reaction: 5 to 20 cycles of PCR (94° C. 30 seconds, 60° C. 30 seconds, 72° C. 30 seconds), during which time only one strand of the double-stranded PCR template is synthesized. Each cycle of PCR is expected to produce one copy of the aa-labeled, single-stranded aDNA. This PCR product is then purified and a Cy3 or Cy5 label is incorporated by standard chemical coupling.

3. Incorporation of aminoallyl modified dUTP (aadUTP) during PCR with an aDNA template using forward and reverse primers to yield aa-labeled, double-stranded aDNA.

Methods: 1 μg of the aDNA PCR product, generated using the NSR7 primer pool as described in Example 11, is added to a PCR reaction mix as follows:
100 to 1000 μM aadUTP+dCTP+cATP+dGTP+dUTP (the optimal balance of aadUTP to dUTP may be empirically determined using routine experimentation)
4 mM MgCl₂
400-1000 mM of the forward and reverse primer (e.g., Forward: SEQ ID NO:949; or Reverse: SEQ ID NO:948)

PCR Reaction: 5 to 20 cycles of PCR (94° C. 30 seconds, 60° C. 30 seconds, 72° C. 30 seconds), during which time both strands of the double-stranded PCR template are synthesized. The double-stranded, aa-labeled aDNA PCR product is then purified and a Cy3 or Cy5 label is incorporated by standard chemical coupling.

While the preferred embodiment of the invention has been illustrated and described, it will be appreciated that various changes can be made therein without departing from the scope of the invention.

The embodiments of the invention in which an exclusive property or privilege is claimed are defined as follows:

1. A method of selectively amplifying a target population of nucleic acid molecules, the method comprising the step of using a population of oligonucleotides to prime the amplification of a target population of nucleic acid molecules within a larger population of nucleic acid molecules, wherein:
(a) each oligonucleotide comprises a hybridizing portion, wherein the hybridizing portion consists of one of 6, 7, or 8 nucleotides; and
(b) the population of oligonucleotides is selected to hybridize under defined conditions to a first subpopulation of the target nucleic acid population, but not hybridize under the defined conditions to a second subpopulation of the target nucleic acid population.

2. The method of claim 1, wherein each oligonucleotide further comprises a defined sequence portion located 5′ to the hybridizing portion.

3. The method of claim 2, wherein the defined sequence portion comprises a transcriptional promoter.

4. The method of claim 2, wherein the defined sequence portion comprises a primer binding site for PCR amplification.

5. The method of claim 1, wherein the population of hybridizing portions is selected from all possible oligonucleotides having a length of 6 nucleotides, 7 nucleotides, or 8 nucleotides.

6. The method of claim 1, wherein the population of hybridizing portions is selected from all possible oligonucleotides having a length of 6 nucleotides.

7. The method of claim 1, wherein the population of hybridizing portions is selected from the oligonucleotides comprising SEQ ID NOS:1-933.

8. The method of claim 1, wherein the hybridizing portion is a member of the population of oligonucleotides comprising SEQ ID NOS:1-933.

9. The method of claim 1, wherein the population of hybridizing portions is selected from all possible oligonucleotides having a length of 7 nucleotides.

10. The method of claim 1, wherein the population of hybridizing portions is selected from all possible oligonucleotides having a length of 8 nucleotides.

11. The method of claim 1, wherein the target population of nucleic acid molecules is obtained from mammalian blood cells.

12. The method of claim 11, wherein the mammalian blood cells are selected from the group consisting of neutrophils, eosinophils, basophils, lymphocytes, monocytes, erythrocytes, and platelets.

13. The method of claim 1, wherein the target population of nucleic acid molecules comprises mRNA molecules obtained from mammalian blood cells.

14. The method of claim 1, wherein the second subpopulation of the target nucleic acid population consists essentially of the most abundant nucleic acid molecules in the target population of nucleic acid molecules.

15. The method of claim 14, wherein the most abundant nucleic acid molecules are selected from the group consisting of ribosomal RNA, ribosomal DNA, globin DNA, or globin RNA.

16. The method of claim 3, wherein the amplification of the target population of nucleic acid molecules comprises the step of reverse transcribing the target population of nucleic acid molecules followed by in vitro transcription to generate RNA molecules.

17. The method of claim 4, wherein the amplification of the target population further comprises the step of reverse transcribing the target population of nucleic acid molecules followed by PCR amplification to generate double-stranded DNA molecules.

18. The method of claim 16 or 17, wherein the reverse transcription step is carried out in the presence of dNTPs, wherein the dNTP concentration comprises a range of from 50 to 5000 micromolar.

19. The method of claim 18, wherein the dNTP concentration comprises a range of from 1000 to 2000 micromolar.

20. The method of claim 1, wherein the defined hybridization conditions comprise an annealing temperature in the range of from 40° C. to 50° C.

21. The method of claim 2, wherein a spacer portion is disposed between the defined sequence portion and the hybridizing portion of each oligonucleotide.

22. A method of selectively amplifying a target population of nucleic acid molecules, the method comprising the step of using a population of oligonucleotides to prime the amplification of a target population of nucleic acid molecules within a larger population of nucleic acid molecules, wherein:
(a) each oligonucleotide comprises a hybridizing portion, wherein the hybridizing portion consists of one of 6, 7, or 8 nucleotides and a defined sequence portion located 5′ to the hybridizing portion; and
(b) the population of oligonucleotides is selected to hybridize under defined conditions to a first subpopulation of a target nucleic acid population, but not hybridize under the defined conditions to a second subpopulation of the target nucleic acid population.

23. The method of claim 22, wherein the defined sequence portion comprises a transcriptional promoter suitable for in vitro transcription.

24. The method of claim 22, wherein the defined sequence portion comprises a primer binding site for PCR amplification.

25. A method of selectively amplifying a target population of nucleic acid molecules, the method comprising the step of using a population of oligonucleotides to prime the amplification of a target population of nucleic acid molecules within a larger population of nucleic acid molecules, wherein each oligonucleotide within the population of oligonucleotides comprises a hybridizing portion and a defined sequence portion located 5′ to the hybridizing portion, wherein the hybridizing portion is a member of the population of oligonucleotides comprising SEQ ID NOS:1-933.

26. The method of claim 25, wherein the defined sequence portion comprises a transcriptional promoter suitable for in vitro transcription.

27. The method of claim 25, wherein the population of hybridizing portions comprises at least 10% of the oligonucleotides comprising SEQ ID NOS:1-933.

28. A population of oligonucleotides comprising SEQ ID NOS:1-933.

29. A population of oligonucleotides consisting of SEQ ID NOS:1-933.

30. A population of oligonucleotides comprising at least 10% of the oligonucleotides comprising SEQ ID NOS:1-933.

31. A reagent for selectively amplifying a target population of nucleic acid molecules, the reagent comprising at least 10% of the oligonucleotides comprising SEQ ID NOS:1-933.

32. A reagent for selectively amplifying a target population of nucleic acid molecules, the reagent comprising a population of oligonucleotides to prime the amplification of a target population of nucleic acid molecules, wherein each oligonucleotide comprises a hybridizing portion and a defined sequence portion located 5′ to the hybridizing portion, wherein the hybridizing portion is a member of the population of oligonucleotides comprising SEQ ID NOS:1-933.

33. The reagent of claim 32, wherein the defined sequence portion comprises a transcriptional promoter suitable for in vitro transcription.

34. The reagent of claim 32, wherein the population of hybridizing portions comprises at least 10% of the oligonucleotides comprising SEQ ID NOS:1-933.

35. The reagent of claim 32, wherein the population of hybridizing portions comprises the oligonucleotides consisting of SEQ ID NOS:1-933.

36. A kit for selectively amplifying a target population of nucleic acid molecules, the kit comprising a reagent comprising a population of oligonucleotides, wherein each oligonucleotide comprises a hybridizing portion that is a member of the population of oligonucleotides comprising SEQ ID NOS:1-933.

37. The kit of claim 36, wherein the population of hybridizing portions comprises at least 10% of the oligonucleotides comprising SEQ ID NOS:1-933.

38. The kit of claim 36, wherein the population of hybridizing portions comprises the oligonucleotides consisting of SEQ ID NOS:1-933.

39. The kit of claim 36, further comprising at least one of the following components: a reverse transcriptase, a DNA polymerase, a DNA ligase, a RNase H enzyme, a Tris buffer, a potassium salt, a magnesium salt, an ammonium salt, a reducing agent, deoxynucleoside triphosphates, or a ribonuclease inhibitor.

40. The kit of claim 36, further comprising at least one of the following components: an RNA polymerase, an IPPase, a transcription buffer, a Tris buffer, a sodium salt, a magnesium salt, spermidine, a reducing agent, nucleoside triphosphates, or amino-allyl-UTP.

41. A method of selectively amplifying a target population of nucleic acid molecules, the method comprising the steps of:
(a) providing a population of oligonucleotides, wherein each oligonucleotide comprises a hybridizing portion and transcriptional promoter portion located 5′ to the hybridizing portion, wherein the hybridizing portion is a member of the population of oligonucleotides comprising SEQ ID NOS:1-933;
(b) annealing the population of oligonucleotides to a sample comprising mRNA isolated from a mammalian subject;
(c) synthesizing cDNA from the mRNA using a reverse transcriptase enzyme;
(d) synthesizing double stranded cDNA using a DNA polymerase; and
(e) transcribing the double-stranded cDNA into RNA using an RNA polymerase that binds to the transcriptional promoter portion of each oligonucleotide to generate amplified RNA.

42. A method of selectively amplifying a target population of nucleic acid molecules, the method comprising the steps of:
(a) providing a first population of oligonucleotides, wherein each oligonucleotide comprises a hybridizing portion and a first PCR primer binding site located 5′ to the hybridizing portion, wherein the hybridizing portion is a member of the population of oligonucleotides comprising SEQ ID NOS:1-933;
(b) annealing the population of oligonucleotides to a sample comprising mRNA isolated from a mammalian subject;
(c) synthesizing cDNA from the mRNA using a reverse transcriptase enzyme;
(d) synthesizing double stranded cDNA using a DNA polymerase and a second population of oligonucleotides, wherein each oligonucleotide comprises a random hybridizing portion and a second PCR binding site located 5′ to the hybridizing portion; and
(e) PCR amplifying the double stranded cDNA using thermostable DNA polymerase, a first PCR primer that binds to the first PCR primer binding site and a second PCR primer that binds to the second PCR primer binding site to generate amplified double stranded DNA.


SEQUENCE LISTING (959 sequences)

SEQ ID NO: 1 - DNA, 6 bp, Artificial Sequence
  oligonucleotide 6-mer sequences
cgtttt

SEQ ID NO: 2 - DNA, 6 bp, Artificial Sequence
  oligonucleotide 6-mer sequences
agtttt

SEQ ID NO: 3 - DNA, 6 bp, Artificial Sequence
  oligonucleotide 6-mer sequences
gcgttt

SEQ ID NO: 4 - DNA, 6 bp, Artificial Sequence
  oligonucleotide 6-mer sequences
acgttt

SEQ ID NO: 5 - DNA, 6 bp, Artificial Sequence
  oligonucleotide 6-mer sequences
ctcttt

SEQ ID NO: 6 - DNA, 6 bp, Artificial Sequence
  oligonucleotide 6-mer sequences
tacttt

SEQ ID NO: 7 - DNA, 6 bp, Artificial Sequence
  oligonucleotide 6-mer sequences
ctattt

SEQ ID NO: 8 - DNA, 6 bp, Artificial Sequence
  oligonucleotide 6-mer sequences
tcattt

SEQ ID NO: 9 - DNA, 6 bp, Artificial Sequence
  oligonucleotide 6-mer sequences
caattt

SEQ ID NO: 10 - DNA, 6 bp, Artificial Sequence
  oligonucleotide 6-mer sequences
aaattt

SEQ ID NO: 11 - DNA, 6 bp, Artificial Sequence
  oligonucleotide 6-mer sequences
ggtgtt

SEQ ID NO: 12 - DNA, 6 bp, Artificial Sequence
  oligonucleotide 6-mer sequences
agtgtt

SEQ ID NO: 13 - DNA, 6 bp, Artificial Sequence
  oligonucleotide 6-mer sequences
tatgtt

SEQ ID NO: 14 - DNA, 6 bp, Artificial Sequence
  oligonucleotide 6-mer sequences
gatgtt

SEQ ID NO: 15 - DNA, 6 bp, Artificial Sequence
  oligonucleotide 6-mer sequences
ccggtt

SEQ ID NO: 16 - DNA, 6 bp, Artificial Sequence
  oligonucleotide 6-mer sequences
aaggtt

SEQ ID NO: 17 - DNA, 6 bp, Artificial Sequence
  oligonucleotide 6-mer sequences
gtcgtt

SEQ ID NO: 18 - DNA, 6 bp, Artificial Sequence
  oligonucleotide 6-mer sequences
agcgtt

SEQ ID NO: 19 - DNA, 6 bp, Artificial Sequence
  oligonucleotide 6-mer sequences
tccgtt

SEQ ID NO: 20 - DNA, 6 bp, Artificial Sequence
  oligonucleotide 6-mer sequences
tacgtt

SEQ ID NO: 21 - DNA, 6 bp, Artificial Sequence
  oligonucleotide 6-mer sequences
aacgtt

SEQ ID NO: 22 - DNA, 6 bp, Artificial Sequence
  oligonucleotide 6-mer sequences
cgagtt

SEQ ID NO: 23 - DNA, 6 bp, Artificial Sequence
  oligonucleotide 6-mer sequences
tgtctt

SEQ ID NO: 24 - DNA, 6 bp, Artificial Sequence
  oligonucleotide 6-mer sequences
actctt

SEQ ID NO: 25 - DNA, 6 bp, Artificial Sequence
  oligonucleotide 6-mer sequences
tatctt

SEQ ID NO: 26 - DNA, 6 bp, Artificial Sequence
  oligonucleotide 6-mer sequences
gatctt

SEQ ID NO: 27 - DNA, 6 bp, Artificial Sequence
  oligonucleotide 6-mer sequences
gtcctt

SEQ ID NO: 28 - DNA, 6 bp, Artificial Sequence
  oligonucleotide 6-mer sequences
cccctt

SEQ ID NO: 29 - DNA, 6 bp, Artificial Sequence
  oligonucleotide 6-mer sequences
ctactt

SEQ ID NO: 30 - DNA, 6 bp, Artificial Sequence
  oligonucleotide 6-mer sequences
acactt

SEQ ID NO: 31 - DNA, 6 bp, Artificial Sequence
  oligonucleotide 6-mer sequences
cgtatt

SEQ ID NO: 32 - DNA, 6 bp, Artificial Sequence
  oligonucleotide 6-mer sequences
agtatt

SEQ ID NO: 33 - DNA, 6 bp, Artificial Sequence
  oligonucleotide 6-mer sequences
tctatt

SEQ ID NO: 34 - DNA, 6 bp, Artificial Sequence
  oligonucleotide 6-mer sequences
actatt

SEQ ID NO: 35 - DNA, 6 bp, Artificial Sequence
  oligonucleotide 6-mer sequences
tatatt

SEQ ID NO: 36 - DNA, 6 bp, Artificial Sequence
  oligonucleotide 6-mer sequences
gatatt

SEQ ID NO: 37 - DNA, 6 bp, Artificial Sequence
  oligonucleotide 6-mer sequences
catatt

SEQ ID NO: 38 - DNA, 6 bp, Artificial Sequence
  oligonucleotide 6-mer sequences
gtgatt

SEQ ID NO: 39 - DNA, 6 bp, Artificial Sequence
  oligonucleotide 6-mer sequences
atgatt

SEQ ID NO: 40 - DNA, 6 bp, Artificial Sequence
  oligonucleotide 6-mer sequences
gcgatt

SEQ ID NO: 41 - DNA, 6 bp, Artificial Sequence
  oligonucleotide 6-mer sequences
acgatt

SEQ ID NO: 42 - DNA, 6 bp, Artificial Sequence
  oligonucleotide 6-mer sequences
cagatt

SEQ ID NO: 43 - DNA, 6 bp, Artificial Sequence
  oligonucleotide 6-mer sequences
aagatt

SEQ ID NO: 44 - DNA, 6 bp, Artificial Sequence
  oligonucleotide 6-mer sequences
gtcatt

SEQ ID NO: 45 - DNA, 6 bp, Artificial Sequence
  oligonucleotide 6-mer sequences
agcatt

SEQ ID NO: 46 - DNA, 6 bp, Artificial Sequence
  oligonucleotide 6-mer sequences
accatt

SEQ ID NO: 47 - DNA, 6 bp, Artificial Sequence
  oligonucleotide 6-mer sequences
gcaatt

SEQ ID NO: 48 - DNA, 6 bp, Artificial Sequence
  oligonucleotide 6-mer sequences
acaatt

SEQ ID NO: 49 - DNA, 6 bp, Artificial Sequence
  oligonucleotide 6-mer sequences
aaaatt

SEQ ID NO: 50 - DNA, 6 bp, Artificial Sequence
  oligonucleotide 6-mer sequences
ttttgt

SEQ ID NO: 51 - DNA, 6 bp, Artificial Sequence
  oligonucleotide 6-mer sequences
gtttgt

SEQ ID NO: 52 - DNA, 6 bp, Artificial Sequence
  oligonucleotide 6-mer sequences
tgttgt

SEQ ID NO: 53 - DNA, 6 bp, Artificial Sequence
  oligonucleotide 6-mer sequences
cgttgt

SEQ ID NO: 54 - DNA, 6 bp, Artificial Sequence
  oligonucleotide 6-mer sequences
gattgt

SEQ ID NO: 55 - DNA, 6 bp, Artificial Sequence
  oligonucleotide 6-mer sequences
aattgt

SEQ ID NO: 56 - DNA, 6 bp, Artificial Sequence
  oligonucleotide 6-mer sequences
ctgtgt

SEQ ID NO: 57 - DNA, 6 bp, Artificial Sequence
  oligonucleotide 6-mer sequences
atgtgt

SEQ ID NO: 58 - DNA, 6 bp, Artificial Sequence
  oligonucleotide 6-mer sequences
gggtgt

SEQ ID NO: 59 - DNA, 6 bp, Artificial Sequence
  oligonucleotide 6-mer sequences
tcgtgt

SEQ ID NO: 60 - DNA, 6 bp, Artificial Sequence
  oligonucleotide 6-mer sequences
gcgtgt

SEQ ID NO: 61 - DNA, 6 bp, Artificial Sequence
  oligonucleotide 6-mer sequences
tagtgt

SEQ ID NO: 62 - DNA, 6 bp, Artificial Sequence
  oligonucleotide 6-mer sequences
cgctgt

SEQ ID NO: 63 - DNA, 6 bp, Artificial Sequence
  oligonucleotide 6-mer sequences
gcctgt

SEQ ID NO: 64 - DNA, 6 bp, Artificial Sequence
  oligonucleotide 6-mer sequences
tactgt

SEQ ID NO: 65 - DNA, 6 bp, Artificial Sequence
  oligonucleotide 6-mer sequences
gactgt

SEQ ID NO: 66 - DNA, 6 bp, Artificial Sequence
  oligonucleotide 6-mer sequences
aactgt

SEQ ID NO: 67 - DNA, 6 bp, Artificial Sequence
  oligonucleotide 6-mer sequences
gtatgt

SEQ ID NO: 68 - DNA, 6 bp, Artificial Sequence
  oligonucleotide 6-mer sequences
ctatgt

SEQ ID NO: 69 - DNA, 6 bp, Artificial Sequence
  oligonucleotide 6-mer sequences
atatgt

SEQ ID NO: 70 - DNA, 6 bp, Artificial Sequence
  oligonucleotide 6-mer sequences
tgatgt

SEQ ID NO: 71 - DNA, 6 bp, Artificial Sequence
  oligonucleotide 6-mer sequences
cgatgt

SEQ ID NO: 72 - DNA, 6 bp, Artificial Sequence
  oligonucleotide 6-mer sequences
agatgt

SEQ ID NO: 73 - DNA, 6 bp, Artificial Sequence
  oligonucleotide 6-mer sequences
taatgt

SEQ ID NO: 74 - DNA, 6 bp, Artificial Sequence
  oligonucleotide 6-mer sequences
gaatgt

SEQ ID NO: 75 - DNA, 6 bp, Artificial Sequence
  oligonucleotide 6-mer sequences
caatgt

SEQ ID NO: 76 - DNA, 6 bp, Artificial Sequence
  oligonucleotide 6-mer sequences
attggt

SEQ ID NO: 77 - DNA, 6 bp, Artificial Sequence
  oligonucleotide 6-mer sequences
gctggt

SEQ ID NO: 78 - DNA, 6 bp, Artificial Sequence
  oligonucleotide 6-mer sequences
ttgggt

SEQ ID NO: 79 - DNA, 6 bp, Artificial Sequence
  oligonucleotide 6-mer sequences
gagggt

SEQ ID NO: 80 - DNA, 6 bp, Artificial Sequence
  oligonucleotide 6-mer sequences
aagggt

SEQ ID NO: 81 - DNA, 6 bp, Artificial Sequence
  oligonucleotide 6-mer sequences
gtcggt

SEQ ID NO: 82 - DNA, 6 bp, Artificial Sequence
  oligonucleotide 6-mer sequences
ctcggt

SEQ ID NO: 83 - DNA, 6 bp, Artificial Sequence
  oligonucleotide 6-mer sequences
tccggt

SEQ ID NO: 84 - DNA, 6 bp, Artificial Sequence
  oligonucleotide 6-mer sequences
accggt

SEQ ID NO: 85 - DNA, 6 bp, Artificial Sequence
  oligonucleotide 6-mer sequences
ttaggt

SEQ ID NO: 86 - DNA, 6 bp, Artificial Sequence
  oligonucleotide 6-mer sequences
ctaggt

SEQ ID NO: 87 - DNA, 6 bp, Artificial Sequence
  oligonucleotide 6-mer sequences
agaggt

SEQ ID NO: 88 - DNA, 6 bp, Artificial Sequence
  oligonucleotide 6-mer sequences
taaggt

SEQ ID NO: 89 - DNA, 6 bp, Artificial Sequence
  oligonucleotide 6-mer sequences
gaaggt

SEQ ID NO: 90 - DNA, 6 bp, Artificial Sequence
  oligonucleotide 6-mer sequences
caaggt

SEQ ID NO: 91 - DNA, 6 bp, Artificial Sequence
  oligonucleotide 6-mer sequences
cttcgt

SEQ ID NO: 92 - DNA, 6 bp, Artificial Sequence
  oligonucleotide 6-mer sequences
cgtcgt

SEQ ID NO: 93 - DNA, 6 bp, Artificial Sequence
  oligonucleotide 6-mer sequences
agtcgt

SEQ ID NO: 94 - DNA, 6 bp, Artificial Sequence
  oligonucleotide 6-mer sequences
aatcgt

SEQ ID NO: 95 - DNA, 6 bp, Artificial Sequence
  oligonucleotide 6-mer sequences
ttgcgt

SEQ ID NO: 96 - DNA, 6 bp, Artificial Sequence
  oligonucleotide 6-mer sequences
atgcgt

SEQ ID NO: 97 - DNA, 6 bp, Artificial Sequence
  oligonucleotide 6-mer sequences
ccgcgt

SEQ ID NO: 98 - DNA, 6 bp, Artificial Sequence
  oligonucleotide 6-mer sequences
acgcgt

SEQ ID NO: 99 - DNA, 6 bp, Artificial Sequence
  oligonucleotide 6-mer sequences
aagcgt

SEQ ID NO: 100 - DNA, 6 bp, Artificial Sequence
  oligonucleotide 6-mer sequences
ctccgt

SEQ ID NO: 101 - DNA, 6 bp, Artificial Sequence
  oligonucleotide 6-mer sequences
atccgt

SEQ ID NO: 102 - DNA, 6 bp, Artificial Sequence
  oligonucleotide 6-mer sequences
aaccgt

SEQ ID NO: 103 - DNA, 6 bp, Artificial Sequence
  oligonucleotide 6-mer sequences
ttacgt

SEQ ID NO: 104 - DNA, 6 bp, Artificial Sequence
  oligonucleotide 6-mer sequences
gtacgt

SEQ ID NO: 105 - DNA, 6 bp, Artificial Sequence
  oligonucleotide 6-mer sequences
atacgt

SEQ ID NO: 106 - DNA, 6 bp, Artificial Sequence
  oligonucleotide 6-mer sequences
ggacgt

SEQ ID NO: 107 - DNA, 6 bp, Artificial Sequence
  oligonucleotide 6-mer sequences
taacgt

SEQ ID NO: 108 - DNA, 6 bp, Artificial Sequence
  oligonucleotide 6-mer sequences
caacgt

SEQ ID NO: 109 - DNA, 6 bp, Artificial Sequence
  oligonucleotide 6-mer sequences
aaacgt

SEQ ID NO: 110 - DNA, 6 bp, Artificial Sequence
  oligonucleotide 6-mer sequences
tgtagt

SEQ ID NO: 111 - DNA, 6 bp, Artificial Sequence
  oligonucleotide 6-mer sequences
cgtagt

SEQ ID NO: 112 - DNA, 6 bp, Artificial Sequence
  oligonucleotide 6-mer sequences
tctagt

SEQ ID NO: 113 - DNA, 6 bp, Artificial Sequence
  oligonucleotide 6-mer sequences
cctagt

SEQ ID NO: 114 - DNA, 6 bp, Artificial Sequence
  oligonucleotide 6-mer sequences
tatagt

SEQ ID NO: 115 - DNA, 6 bp, Artificial Sequence
  oligonucleotide 6-mer sequences
aatagt

SEQ ID NO: 116 - DNA, 6 bp, Artificial Sequence
  oligonucleotide 6-mer sequences
atgagt

SEQ ID NO: 117 - DNA, 6 bp, Artificial Sequence
  oligonucleotide 6-mer sequences
cggagt

SEQ ID NO: 118 - DNA, 6 bp, Artificial Sequence
  oligonucleotide 6-mer sequences
tcgagt

SEQ ID NO: 119 - DNA, 6 bp, Artificial Sequence
  oligonucleotide 6-mer sequences
ccgagt

SEQ ID NO: 120 - DNA, 6 bp, Artificial Sequence
  oligonucleotide 6-mer sequences
acgagt

SEQ ID NO: 121 - DNA, 6 bp, Artificial Sequence
  oligonucleotide 6-mer sequences
agcagt

SEQ ID NO: 122 - DNA, 6 bp, Artificial Sequence
  oligonucleotide 6-mer sequences
tacagt

SEQ ID NO: 123 - DNA, 6 bp, Artificial Sequence
  oligonucleotide 6-mer sequences
gtaagt

SEQ ID NO: 124 - DNA, 6 bp, Artificial Sequence
  oligonucleotide 6-mer sequences
ataagt

SEQ ID NO: 125 - DNA, 6 bp, Artificial Sequence
  oligonucleotide 6-mer sequences
gcaagt

SEQ ID NO: 126 - DNA, 6 bp, Artificial Sequence
  oligonucleotide 6-mer sequences
acaagt

SEQ ID NO: 127 - DNA, 6 bp, Artificial Sequence
  oligonucleotide 6-mer sequences
tgttct

SEQ ID NO: 128 - DNA, 6 bp, Artificial Sequence
  oligonucleotide 6-mer sequences
ttgtct

SEQ ID NO: 129 - DNA, 6 bp, Artificial Sequence
  oligonucleotide 6-mer sequences
aggtct

SEQ ID NO: 130 - DNA, 6 bp, Artificial Sequence
  oligonucleotide 6-mer sequences
cagtct

SEQ ID NO: 131 - DNA, 6 bp, Artificial Sequence
  oligonucleotide 6-mer sequences
aagtct

SEQ ID NO: 132 - DNA, 6 bp, Artificial Sequence
  oligonucleotide 6-mer sequences
gcctct

SEQ ID NO: 133 - DNA, 6 bp, Artificial Sequence
  oligonucleotide 6-mer sequences
tactct

SEQ ID NO: 134 - DNA, 6 bp, Artificial Sequence
  oligonucleotide 6-mer sequences
gactct

SEQ ID NO: 135 - DNA, 6 bp, Artificial Sequence
  oligonucleotide 6-mer sequences
ctatct

SEQ ID NO: 136 - DNA, 6 bp, Artificial Sequence
  oligonucleotide 6-mer sequences
atatct

SEQ ID NO: 137 - DNA, 6 bp, Artificial Sequence
  oligonucleotide 6-mer sequences
cgatct

SEQ ID NO: 138 - DNA, 6 bp, Artificial Sequence
  oligonucleotide 6-mer sequences
tcatct

SEQ ID NO: 139 - DNA, 6 bp, Artificial Sequence
  oligonucleotide 6-mer sequences
aaatct

SEQ ID NO: 140 - DNA, 6 bp, Artificial Sequence
  oligonucleotide 6-mer sequences
cttgct

SEQ ID NO: 141 - DNA, 6 bp, Artificial Sequence
  oligonucleotide 6-mer sequences
tgtgct

SEQ ID NO: 142 - DNA, 6 bp, Artificial Sequence
  oligonucleotide 6-mer sequences
catgct

SEQ ID NO: 143 - DNA, 6 bp, Artificial Sequence
  oligonucleotide 6-mer sequences
atagct

SEQ ID NO: 144 - DNA, 6 bp, Artificial Sequence
  oligonucleotide 6-mer sequences
acagct

SEQ ID NO: 145 - DNA, 6 bp, Artificial Sequence
  oligonucleotide 6-mer sequences
taagct

SEQ ID NO: 146 - DNA, 6 bp, Artificial Sequence
  oligonucleotide 6-mer sequences
ttgcct

SEQ ID NO: 147 - DNA, 6 bp, Artificial Sequence
  oligonucleotide 6-mer sequences
gtgcct

SEQ ID NO: 148 - DNA, 6 bp, Artificial Sequence
  oligonucleotide 6-mer sequences
atgcct

SEQ ID NO: 149 - DNA, 6 bp, Artificial Sequence
  oligonucleotide 6-mer sequences
acccct

SEQ ID NO: 150 - DNA, 6 bp, Artificial Sequence
  oligonucleotide 6-mer sequences
gtacct

SEQ ID NO: 151 - DNA, 6 bp, Artificial Sequence
  oligonucleotide 6-mer sequences
atacct

SEQ ID NO: 152 - DNA, 6 bp, Artificial Sequence
  oligonucleotide 6-mer sequences
tgacct

SEQ ID NO: 153 - DNA, 6 bp, Artificial Sequence
  oligonucleotide 6-mer sequences
caacct

SEQ ID NO: 154 - DNA, 6 bp, Artificial Sequence
  oligonucleotide 6-mer sequences
attact

SEQ ID NO: 155 - DNA, 6 bp, Artificial Sequence
  oligonucleotide 6-mer sequences
tgtact

SEQ ID NO: 156 - DNA, 6 bp, Artificial Sequence
  oligonucleotide 6-mer sequences
agtact

SEQ ID NO: 157 - DNA, 6 bp, Artificial Sequence
  oligonucleotide 6-mer sequences
tctact

SEQ ID NO: 158 - DNA, 6 bp, Artificial Sequence
  oligonucleotide 6-mer sequences
actact

SEQ ID NO: 159 - DNA, 6 bp, Artificial Sequence
  oligonucleotide 6-mer sequences
tatact

SEQ ID NO: 160 - DNA, 6 bp, Artificial Sequence
  oligonucleotide 6-mer sequences
aatact

SEQ ID NO: 161 - DNA, 6 bp, Artificial Sequence
  oligonucleotide 6-mer sequences
gtgact

SEQ ID NO: 162 - DNA, 6 bp, Artificial Sequence
  oligonucleotide 6-mer sequences
atgact

SEQ ID NO: 163 - DNA, 6 bp, Artificial Sequence
  oligonucleotide 6-mer sequences
gagact

SEQ ID NO: 164 - DNA, 6 bp, Artificial Sequence
  oligonucleotide 6-mer sequences
tacact

SEQ ID NO: 165 - DNA, 6 bp, Artificial Sequence
  oligonucleotide 6-mer sequences
ctaact

SEQ ID NO: 166 - DNA, 6 bp, Artificial Sequence
  oligonucleotide 6-mer sequences
cgaact

SEQ ID NO: 167 - DNA, 6 bp, Artificial Sequence
  oligonucleotide 6-mer sequences
acaact

SEQ ID NO: 168 - DNA, 6 bp, Artificial Sequence
  oligonucleotide 6-mer sequences
taaact

SEQ ID NO: 169 - DNA, 6 bp, Artificial Sequence
  oligonucleotide 6-mer sequences
atttat

SEQ ID NO: 170 - DNA, 6 bp, Artificial Sequence
  oligonucleotide 6-mer sequences
tattat

SEQ ID NO: 171 - DNA, 6 bp, Artificial Sequence
  oligonucleotide 6-mer sequences
gattat

SEQ ID NO: 172 - DNA, 6 bp, Artificial Sequence
  oligonucleotide 6-mer sequences
aattat

SEQ ID NO: 173 - DNA, 6 bp, Artificial Sequence
  oligonucleotide 6-mer sequences
gtgtat

SEQ ID NO: 174 - DNA, 6 bp, Artificial Sequence
  oligonucleotide 6-mer sequences
aggtat

SEQ ID NO: 175 - DNA, 6 bp, Artificial Sequence
  oligonucleotide 6-mer sequences
tcgtat

SEQ ID NO: 176 - DNA, 6 bp, Artificial Sequence
  oligonucleotide 6-mer sequences
gcgtat

SEQ ID NO: 177 - DNA, 6 bp, Artificial Sequence
  oligonucleotide 6-mer sequences
acgtat

SEQ ID NO: 178 - DNA, 6 bp, Artificial Sequence
  oligonucleotide 6-mer sequences
tagtat

SEQ ID NO: 179 - DNA, 6 bp, Artificial Sequence
  oligonucleotide 6-mer sequences
gagtat

SEQ ID NO: 180 - DNA, 6 bp, Artificial Sequence
  oligonucleotide 6-mer sequences
cagtat

SEQ ID NO: 181 - DNA, 6 bp, Artificial Sequence
  oligonucleotide 6-mer sequences
aagtat

SEQ ID NO: 182 - DNA, 6 bp, Artificial Sequence
  oligonucleotide 6-mer sequences
ttctat

SEQ ID NO: 183 - DNA, 6 bp, Artificial Sequence
  oligonucleotide 6-mer sequences
ctctat

SEQ ID NO: 184 - DNA, 6 bp, Artificial Sequence
  oligonucleotide 6-mer sequences
atctat

SEQ ID NO: 185 - DNA, 6 bp, Artificial Sequence
  oligonucleotide 6-mer sequences
tgctat

SEQ ID NO: 186 - DNA, 6 bp, Artificial Sequence
  oligonucleotide 6-mer sequences
acctat

SEQ ID NO: 187 - DNA, 6 bp, Artificial Sequence
  oligonucleotide 6-mer sequences
tactat

SEQ ID NO: 188 - DNA, 6 bp, Artificial Sequence
  oligonucleotide 6-mer sequences
cactat

SEQ ID NO: 189 - DNA, 6 bp, Artificial Sequence
  oligonucleotide 6-mer sequences
aactat

SEQ ID NO: 190 - DNA, 6 bp, Artificial Sequence
  oligonucleotide 6-mer sequences
ttatat

SEQ ID NO: 191 - DNA, 6 bp, Artificial Sequence
  oligonucleotide 6-mer sequences
gtatat

SEQ ID NO: 192 - DNA, 6 bp, Artificial Sequence
  oligonucleotide 6-mer sequences
atatat

SEQ ID NO: 193 - DNA, 6 bp, Artificial Sequence
  oligonucleotide 6-mer sequences
tgatat

SEQ ID NO: 194 - DNA, 6 bp, Artificial Sequence
  oligonucleotide 6-mer sequences
cgatat

SEQ ID NO: 195 - DNA, 6 bp, Artificial Sequence
  oligonucleotide 6-mer sequences
agatat

SEQ ID NO: 196 - DNA, 6 bp, Artificial Sequence
  oligonucleotide 6-mer sequences
tcatat

SEQ ID NO: 197 - DNA, 6 bp, Artificial Sequence
  oligonucleotide 6-mer sequences
ccatat

SEQ ID NO: 198 - DNA, 6 bp, Artificial Sequence
  oligonucleotide 6-mer sequences
acatat

SEQ ID NO: 199 - DNA, 6 bp, Artificial Sequence
  oligonucleotide 6-mer sequences
caatat

SEQ ID NO: 200 - DNA, 6 bp, Artificial Sequence
  oligonucleotide 6-mer sequences
aaatat

SEQ ID NO: 201 - DNA, 6 bp, Artificial Sequence
  oligonucleotide 6-mer sequences
ggtgat

SEQ ID NO: 202 - DNA, 6 bp, Artificial Sequence
  oligonucleotide 6-mer sequences
cgtgat

SEQ ID NO: 203 - DNA, 6 bp, Artificial Sequence
  oligonucleotide 6-mer sequences
tatgat

SEQ ID NO: 204 - DNA, 6 bp, Artificial Sequence
  oligonucleotide 6-mer sequences
gatgat

SEQ ID NO: 205 - DNA, 6 bp, Artificial Sequence
  oligonucleotide 6-mer sequences
gtggat

SEQ ID NO: 206 - DNA, 6 bp, Artificial Sequence
  oligonucleotide 6-mer sequences
tgggat

SEQ ID NO: 207 - DNA, 6 bp, Artificial Sequence
  oligonucleotide 6-mer sequences
agggat

SEQ ID NO: 208 - DNA, 6 bp, Artificial Sequence
  oligonucleotide 6-mer sequences
taggat

SEQ ID NO: 209 - DNA, 6 bp, Artificial Sequence
  oligonucleotide 6-mer sequences
gaggat

SEQ ID NO: 210 - DNA, 6 bp, Artificial Sequence
  oligonucleotide 6-mer sequences
gtcgat

SEQ ID NO: 211 - DNA, 6 bp, Artificial Sequence
  oligonucleotide 6-mer sequences
atcgat

SEQ ID NO: 212 - DNA, 6 bp, Artificial Sequence
  oligonucleotide 6-mer sequences
agcgat

SEQ ID NO: 213 - DNA, 6 bp, Artificial Sequence
  oligonucleotide 6-mer sequences
tccgat

SEQ ID NO: 214 - DNA, 6 bp, Artificial Sequence
  oligonucleotide 6-mer sequences
tacgat

SEQ ID NO: 215 - DNA, 6 bp, Artificial Sequence
  oligonucleotide 6-mer sequences
gacgat

SEQ ID NO: 216 - DNA, 6 bp, Artificial Sequence
  oligonucleotide 6-mer sequences
cacgat

SEQ ID NO: 217 - DNA, 6 bp, Artificial Sequence
  oligonucleotide 6-mer sequences
gtagat

SEQ ID NO: 218 - DNA, 6 bp, Artificial Sequence
  oligonucleotide 6-mer sequences
ggagat

SEQ ID NO: 219 - DNA, 6 bp, Artificial Sequence
  oligonucleotide 6-mer sequences
agagat

SEQ ID NO: 220 - DNA, 6 bp, Artificial Sequence
  oligonucleotide 6-mer sequences
gcagat

SEQ ID NO: 221 - DNA, 6 bp, Artificial Sequence
  oligonucleotide 6-mer sequences
taagat

SEQ ID NO: 222 - DNA, 6 bp, Artificial Sequence
  oligonucleotide 6-mer sequences
gaagat

SEQ ID NO: 223 - DNA, 6 bp, Artificial Sequence
  oligonucleotide 6-mer sequences
tttcat

SEQ ID NO: 224 - DNA, 6 bp, Artificial Sequence
  oligonucleotide 6-mer sequences
tgtcat

SEQ ID NO: 225 - DNA, 6 bp, Artificial Sequence
  oligonucleotide 6-mer sequences
cgtcat

SEQ ID NO: 226 - DNA, 6 bp, Artificial Sequence
  oligonucleotide 6-mer sequences
gctcat

SEQ ID NO: 227 - DNA, 6 bp, Artificial Sequence
  oligonucleotide 6-mer sequences
cctcat

SEQ ID NO: 228 - DNA, 6 bp, Artificial Sequence
  oligonucleotide 6-mer sequences
tatcat

SEQ ID NO: 229 - DNA, 6 bp, Artificial Sequence
  oligonucleotide 6-mer sequences
ttgcat

SEQ ID NO: 230 - DNA, 6 bp, Artificial Sequence
  oligonucleotide 6-mer sequences
gtgcat

SEQ ID NO: 231 - DNA, 6 bp, Artificial Sequence
  oligonucleotide 6-mer sequences
gcgcat

SEQ ID NO: 232 - DNA, 6 bp, Artificial Sequence
  oligonucleotide 6-mer sequences
ctccat

SEQ ID NO: 233 - DNA, 6 bp, Artificial Sequence
  oligonucleotide 6-mer sequences
tcccat

SEQ ID NO: 234 - DNA, 6 bp, Artificial Sequence
  oligonucleotide 6-mer sequences
gaccat

SEQ ID NO: 235 - DNA, 6 bp, Artificial Sequence
  oligonucleotide 6-mer sequences
ttacat

SEQ ID NO: 236 - DNA, 6 bp, Artificial Sequence
  oligonucleotide 6-mer sequences
gtacat

SEQ ID NO: 237 - DNA, 6 bp, Artificial Sequence
  oligonucleotide 6-mer sequences
atacat

SEQ ID NO: 238 - DNA, 6 bp, Artificial Sequence
  oligonucleotide 6-mer sequences
taacat

SEQ ID NO: 239 - DNA, 6 bp, Artificial Sequence
  oligonucleotide 6-mer sequences
gttaat

SEQ ID NO: 240 - DNA, 6 bp, Artificial Sequence
  oligonucleotide 6-mer sequences
tgtaat

SEQ ID NO: 241 - DNA, 6 bp, Artificial Sequence
  oligonucleotide 6-mer sequences
cgtaat

SEQ ID NO: 242 - DNA, 6 bp, Artificial Sequence
  oligonucleotide 6-mer sequences
gctaat

SEQ ID NO: 243 - DNA, 6 bp, Artificial Sequence
  oligonucleotide 6-mer sequences
cctaat

SEQ ID NO: 244 - DNA, 6 bp, Artificial Sequence
  oligonucleotide 6-mer sequences
tataat

SEQ ID NO: 245 - DNA, 6 bp, Artificial Sequence
  oligonucleotide 6-mer sequences
gataat

SEQ ID NO: 246 - DNA, 6 bp, Artificial Sequence
  oligonucleotide 6-mer sequences
atgaat

SEQ ID NO: 247 - DNA, 6 bp, Artificial Sequence
  oligonucleotide 6-mer sequences
aggaat

SEQ ID NO: 248 - DNA, 6 bp, Artificial Sequence
  oligonucleotide 6-mer sequences
ccgaat

SEQ ID NO: 249 - DNA, 6 bp, Artificial Sequence
  oligonucleotide 6-mer sequences
ggcaat

SEQ ID NO: 250 - DNA, 6 bp, Artificial Sequence
  oligonucleotide 6-mer sequences
agcaat

SEQ ID NO: 251 - DNA, 6 bp, Artificial Sequence
  oligonucleotide 6-mer sequences
cccaat

SEQ ID NO: 252 - DNA, 6 bp, Artificial Sequence
  oligonucleotide 6-mer sequences
accaat

SEQ ID NO: 253 - DNA, 6 bp, Artificial Sequence
  oligonucleotide 6-mer sequences
tacaat

SEQ ID NO: 254 - DNA, 6 bp, Artificial Sequence
  oligonucleotide 6-mer sequences
gacaat

SEQ ID NO: 255 - DNA, 6 bp, Artificial Sequence
  oligonucleotide 6-mer sequences
gtaaat

SEQ ID NO: 256 - DNA, 6 bp, Artificial Sequence
  oligonucleotide 6-mer sequences
ctaaat

SEQ ID NO: 257 - DNA, 6 bp, Artificial Sequence
  oligonucleotide 6-mer sequences
taaaat

SEQ ID NO: 258 - DNA, 6 bp, Artificial Sequence
  oligonucleotide 6-mer sequences
aaaaat

SEQ ID NO: 259 - DNA, 6 bp, Artificial Sequence
  oligonucleotide 6-mer sequences
tttttg

SEQ ID NO: 260 - DNA, 6 bp, Artificial Sequence
  oligonucleotide 6-mer sequences
attttg

SEQ ID NO: 261 - DNA, 6 bp, Artificial Sequence
  oligonucleotide 6-mer sequences
ggtttg

SEQ ID NO: 262 - DNA, 6 bp, Artificial Sequence
  oligonucleotide 6-mer sequences
cctttg

SEQ ID NO: 263 - DNA, 6 bp, Artificial Sequence
  oligonucleotide 6-mer sequences
ctgttg

SEQ ID NO: 264 - DNA, 6 bp, Artificial Sequence
  oligonucleotide 6-mer sequences
atgttg

SEQ ID NO: 265 - DNA, 6 bp, Artificial Sequence
  oligonucleotide 6-mer sequences
tggttg

SEQ ID NO: 266 - DNA, 6 bp, Artificial Sequence
  oligonucleotide 6-mer sequences
cggttg

SEQ ID NO: 267 - DNA, 6 bp, Artificial Sequence
  oligonucleotide 6-mer sequences
tcgttg

SEQ ID NO: 268 - DNA, 6 bp, Artificial Sequence
  oligonucleotide 6-mer sequences
atcttg

SEQ ID NO: 269 - DNA, 6 bp, Artificial Sequence
  oligonucleotide 6-mer sequences
atattg

SEQ ID NO: 270 - DNA, 6 bp, Artificial Sequence
  oligonucleotide 6-mer sequences
ggattg

SEQ ID NO: 271 - DNA, 6 bp, Artificial Sequence
  oligonucleotide 6-mer sequences
cgattg

SEQ ID NO: 272 - DNA, 6 bp, Artificial Sequence
  oligonucleotide 6-mer sequences
agattg

SEQ ID NO: 273 - DNA, 6 bp, Artificial Sequence
  oligonucleotide 6-mer sequences
gaattg

SEQ ID NO: 274 - DNA, 6 bp, Artificial Sequence
  oligonucleotide 6-mer sequences
caattg

SEQ ID NO: 275 - DNA, 6 bp, Artificial Sequence
  oligonucleotide 6-mer sequences
tgtgtg

SEQ ID NO: 276 - DNA, 6 bp, Artificial Sequence
  oligonucleotide 6-mer sequences
cgtgtg

SEQ ID NO: 277 - DNA, 6 bp, Artificial Sequence
  oligonucleotide 6-mer sequences
actgtg

SEQ ID NO: 278 - DNA, 6 bp, Artificial Sequence
  oligonucleotide 6-mer sequences
tatgtg

SEQ ID NO: 279 - DNA, 6 bp, Artificial Sequence
  oligonucleotide 6-mer sequences
aatgtg

SEQ ID NO: 280 - DNA, 6 bp, Artificial Sequence
  oligonucleotide 6-mer sequences
agggtg

SEQ ID NO: 281 - DNA, 6 bp, Artificial Sequence
  oligonucleotide 6-mer sequences
tcggtg

SEQ ID NO: 282 - DNA, 6 bp, Artificial Sequence
  oligonucleotide 6-mer sequences
taggtg

SEQ ID NO: 283 - DNA, 6 bp, Artificial Sequence
  oligonucleotide 6-mer sequences
gtcgtg

SEQ ID NO: 284 - DNA, 6 bp, Artificial Sequence
  oligonucleotide 6-mer sequences
tacgtg

SEQ ID NO: 285 - DNA, 6 bp, Artificial Sequence
  oligonucleotide 6-mer sequences
atagtg

SEQ ID NO: 286 - DNA, 6 bp, Artificial Sequence
  oligonucleotide 6-mer sequences
tgagtg

SEQ ID NO: 287 - DNA, 6 bp, Artificial Sequence
  oligonucleotide 6-mer sequences
agagtg

SEQ ID NO: 288 - DNA, 6 bp, Artificial Sequence
  oligonucleotide 6-mer sequences
ccagtg

SEQ ID NO: 289 - DNA, 6 bp, Artificial Sequence
  oligonucleotide 6-mer sequences
taagtg

SEQ ID NO: 290 - DNA, 6 bp, Artificial Sequence
  oligonucleotide 6-mer sequences
caagtg

SEQ ID NO: 291 - DNA, 6 bp, Artificial Sequence
  oligonucleotide 6-mer sequences
actctg

SEQ ID NO: 292 - DNA, 6 bp, Artificial Sequence
  oligonucleotide 6-mer sequences
catctg

SEQ ID NO: 293 - DNA, 6 bp, Artificial Sequence
  oligonucleotide 6-mer sequences
ttgctg

SEQ ID NO: 294 - DNA, 6 bp, Artificial Sequence
  oligonucleotide 6-mer sequences
atgctg

SEQ ID NO: 295 - DNA, 6 bp, Artificial Sequence
  oligonucleotide 6-mer sequences
ttcctg

SEQ ID NO: 296 - DNA, 6 bp, Artificial Sequence
  oligonucleotide 6-mer sequences
tgcctg

SEQ ID NO: 297 - DNA, 6 bp, Artificial Sequence
  oligonucleotide 6-mer sequences
tacctg

SEQ ID NO: 298 - DNA, 6 bp, Artificial Sequence
  oligonucleotide 6-mer sequences
atactg

SEQ ID NO: 299 - DNA, 6 bp, Artificial Sequence
  oligonucleotide 6-mer sequences
tgactg

SEQ ID NO: 300 - DNA, 6 bp, Artificial Sequence
  oligonucleotide 6-mer sequences
agactg

SEQ ID NO: 301 - DNA, 6 bp, Artificial Sequence
  oligonucleotide 6-mer sequences
gaactg

SEQ ID NO: 302 - DNA, 6 bp, Artificial Sequence
  oligonucleotide 6-mer sequences
attatg

SEQ ID NO: 303 - DNA, 6 bp, Artificial Sequence
  oligonucleotide 6-mer sequences
tgtatg

SEQ ID NO: 304 - DNA, 6 bp, Artificial Sequence
  oligonucleotide 6-mer sequences
cgtatg

SEQ ID NO: 305 - DNA, 6 bp, Artificial Sequence
  oligonucleotide 6-mer sequences
agtatg

SEQ ID NO: 306 - DNA, 6 bp, Artificial Sequence
  oligonucleotide 6-mer sequences
tctatg

SEQ ID NO: 307 - DNA, 6 bp, Artificial Sequence
  oligonucleotide 6-mer sequences
cctatg

SEQ ID NO: 308 - DNA, 6 bp, Artificial Sequence
  oligonucleotide 6-mer sequences
actatg

SEQ ID NO: 309 - DNA, 6 bp, Artificial Sequence
  oligonucleotide 6-mer sequences
tatatg

SEQ ID NO: 310 - DNA, 6 bp, Artificial Sequence
  oligonucleotide 6-mer sequences
ttgatg

SEQ ID NO: 311 - DNA, 6 bp, Artificial Sequence
  oligonucleotide 6-mer sequences
gtgatg

SEQ ID NO: 312 - DNA, 6 bp, Artificial Sequence
  oligonucleotide 6-mer sequences
gggatg

SEQ ID NO: 313 - DNA, 6 bp, Artificial Sequence
  oligonucleotide 6-mer sequences
cggatg

SEQ ID NO: 314 - DNA, 6 bp, Artificial Sequence
  oligonucleotide 6-mer sequences
aggatg

SEQ ID NO: 315 - DNA, 6 bp, Artificial Sequence
  oligonucleotide 6-mer sequences
tcgatg

SEQ ID NO: 316 - DNA, 6 bp, Artificial Sequence
  oligonucleotide 6-mer sequences
ccgatg

SEQ ID NO: 317 - DNA, 6 bp, Artificial Sequence
  oligonucleotide 6-mer sequences
acgatg

SEQ ID NO: 318 - DNA, 6 bp, Artificial Sequence
  oligonucleotide 6-mer sequences
gagatg

SEQ ID NO: 319 - DNA, 6 bp, Artificial Sequence
  oligonucleotide 6-mer sequences
aagatg

SEQ ID NO: 320 - DNA, 6 bp, Artificial Sequence
  oligonucleotide 6-mer sequences
cgcatg

SEQ ID NO: 321 - DNA, 6 bp, Artificial Sequence
  oligonucleotide 6-mer sequences
tacatg

SEQ ID NO: 322 - DNA, 6 bp, Artificial Sequence
  oligonucleotide 6-mer sequences
gtaatg

SEQ ID NO: 323 - DNA, 6 bp, Artificial Sequence
  oligonucleotide 6-mer sequences
ctaatg

SEQ ID NO: 324 - DNA, 6 bp, Artificial Sequence
  oligonucleotide 6-mer sequences
ataatg

SEQ ID NO: 325 - DNA, 6 bp, Artificial Sequence
  oligonucleotide 6-mer sequences
tgaatg

SEQ ID NO: 326 - DNA, 6 bp, Artificial Sequence
  oligonucleotide 6-mer sequences
ggaatg

SEQ ID NO: 327 - DNA, 6 bp, Artificial Sequence
  oligonucleotide 6-mer sequences
agaatg

SEQ ID NO: 328 - DNA, 6 bp, Artificial Sequence
  oligonucleotide 6-mer sequences
gcaatg

SEQ ID NO: 329 - DNA, 6 bp, Artificial Sequence
  oligonucleotide 6-mer sequences
aaaatg

SEQ ID NO: 330 - DNA, 6 bp, Artificial Sequence
  oligonucleotide 6-mer sequences
ttttgg

SEQ ID NO: 331 - DNA, 6 bp, Artificial Sequence
  oligonucleotide 6-mer sequences
gtttgg

SEQ ID NO: 332 - DNA, 6 bp, Artificial Sequence
  oligonucleotide 6-mer sequences
tgttgg

SEQ ID NO: 333 - DNA, 6 bp, Artificial Sequence
  oligonucleotide 6-mer sequences
gattgg

SEQ ID NO: 334 - DNA, 6 bp, Artificial Sequence
  oligonucleotide 6-mer sequences
tcgtgg

SEQ ID NO: 335 - DNA, 6 bp, Artificial Sequence
  oligonucleotide 6-mer sequences
aactgg

SEQ ID NO: 336 - DNA, 6 bp, Artificial Sequence
  oligonucleotide 6-mer sequences
ctatgg

SEQ ID NO: 337 - DNA, 6 bp, Artificial Sequence
  oligonucleotide 6-mer sequences
ggatgg

SEQ ID NO: 338 - DNA, 6 bp, Artificial Sequence
  oligonucleotide 6-mer sequences
cgatgg

SEQ ID NO: 339 - DNA, 6 bp, Artificial Sequence
  oligonucleotide 6-mer sequences
taatgg

SEQ ID NO: 340 - DNA, 6 bp, Artificial Sequence
  oligonucleotide 6-mer sequences
attggg

SEQ ID NO: 341 - DNA, 6 bp, Artificial Sequence
  oligonucleotide 6-mer sequences
gatggg

SEQ ID NO: 342 - DNA, 6 bp, Artificial Sequence
  oligonucleotide 6-mer sequences
tagggg

SEQ ID NO: 343 - DNA, 6 bp, Artificial Sequence
  oligonucleotide 6-mer sequences
ttgcgg

SEQ ID NO: 344 - DNA, 6 bp, Artificial Sequence
  oligonucleotide 6-mer sequences
atgcgg

SEQ ID NO: 345 - DNA, 6 bp, Artificial Sequence
  oligonucleotide 6-mer sequences
aagcgg

SEQ ID NO: 346 - DNA, 6 bp, Artificial Sequence
  oligonucleotide 6-mer sequences
aaccgg

SEQ ID NO: 347 - DNA, 6 bp, Artificial Sequence
  oligonucleotide 6-mer sequences
atacgg

SEQ ID NO: 348 - DNA, 6 bp, Artificial Sequence
  oligonucleotide 6-mer sequences
tttagg

SEQ ID NO: 349 - DNA, 6 bp, Artificial Sequence
  oligonucleotide 6-mer sequences
gttagg

SEQ ID NO: 350 - DNA, 6 bp, Artificial Sequence
  oligonucleotide 6-mer sequences
tgtagg

SEQ ID NO: 351 - DNA, 6 bp, Artificial Sequence
  oligonucleotide 6-mer sequences
tatagg

SEQ ID NO: 352 - DNA, 6 bp, Artificial Sequence
  oligonucleotide 6-mer sequences
catagg

SEQ ID NO: 353 - DNA, 6 bp, Artificial Sequence
  oligonucleotide 6-mer sequences
atgagg

SEQ ID NO: 354 - DNA, 6 bp, Artificial Sequence
  oligonucleotide 6-mer sequences
aagagg

SEQ ID NO: 355 - DNA, 6 bp, Artificial Sequence
  oligonucleotide 6-mer sequences
ttaagg

SEQ ID NO: 356 - DNA, 6 bp, Artificial Sequence
  oligonucleotide 6-mer sequences
gtaagg

SEQ ID NO: 357 - DNA, 6 bp, Artificial Sequence
  oligonucleotide 6-mer sequences
ataagg

SEQ ID NO: 358 - DNA, 6 bp, Artificial Sequence
  oligonucleotide 6-mer sequences
tgaagg

SEQ ID NO: 359 - DNA, 6 bp, Artificial Sequence
  oligonucleotide 6-mer sequences
acaagg

SEQ ID NO: 360 - DNA, 6 bp, Artificial Sequence
  oligonucleotide 6-mer sequences
gaaagg

SEQ ID NO: 361 - DNA, 6 bp, Artificial Sequence
  oligonucleotide 6-mer sequences
atttcg

SEQ ID NO: 362 - DNA, 6 bp, Artificial Sequence
  oligonucleotide 6-mer sequences
tgttcg

SEQ ID NO: 363 - DNA, 6 bp, Artificial Sequence
  oligonucleotide 6-mer sequences
aattcg

SEQ ID NO: 364 - DNA, 6 bp, Artificial Sequence
  oligonucleotide 6-mer sequences
ctgtcg

SEQ ID NO: 365 - DNA, 6 bp, Artificial Sequence
  oligonucleotide 6-mer sequences
tagtcg

SEQ ID NO: 366 - DNA, 6 bp, Artificial Sequence
  oligonucleotide 6-mer sequences
gagtcg

SEQ ID NO: 367 - DNA, 6 bp, Artificial Sequence
  oligonucleotide 6-mer sequences
tgctcg

SEQ ID NO: 368 - DNA, 6 bp, Artificial Sequence
  oligonucleotide 6-mer sequences
atatcg

SEQ ID NO: 369 - DNA, 6 bp, Artificial Sequence
  oligonucleotide 6-mer sequences
tcatcg

SEQ ID NO: 370 - DNA, 6 bp, Artificial Sequence
  oligonucleotide 6-mer sequences
taatcg

SEQ ID NO: 371 - DNA, 6 bp, Artificial Sequence
  oligonucleotide 6-mer sequences
gttgcg

SEQ ID NO: 372 - DNA, 6 bp, Artificial Sequence
  oligonucleotide 6-mer sequences
tatgcg

SEQ ID NO: 373 - DNA, 6 bp, Artificial Sequence
  oligonucleotide 6-mer sequences
gatgcg

SEQ ID NO: 374 - DNA, 6 bp, Artificial Sequence
  oligonucleotide 6-mer sequences
ctggcg

SEQ ID NO: 375 - DNA, 6 bp, Artificial Sequence
  oligonucleotide 6-mer sequences
aaggcg

SEQ ID NO: 376 - DNA, 6 bp, Artificial Sequence
  oligonucleotide 6-mer sequences
tacgcg

SEQ ID NO: 377 - DNA, 6 bp, Artificial Sequence
  oligonucleotide 6-mer sequences
aacgcg

SEQ ID NO: 378 - DNA, 6 bp, Artificial Sequence
  oligonucleotide 6-mer sequences
atagcg

SEQ ID NO: 379 - DNA, 6 bp, Artificial Sequence
  oligonucleotide 6-mer sequences
taagcg

SEQ ID NO: 380 - DNA, 6 bp, Artificial Sequence
  oligonucleotide 6-mer sequences
catccg

SEQ ID NO: 381 - DNA, 6 bp, Artificial Sequence
  oligonucleotide 6-mer sequences
aatccg

SEQ ID NO: 382 - DNA, 6 bp, Artificial Sequence
  oligonucleotide 6-mer sequences
atgccg

SEQ ID NO: 383 - DNA, 6 bp, Artificial Sequence
  oligonucleotide 6-mer sequences
aggccg

SEQ ID NO: 384 - DNA, 6 bp, Artificial Sequence
  oligonucleotide 6-mer sequences
ataccg

SEQ ID NO: 385 - DNA, 6 bp, Artificial Sequence
  oligonucleotide 6-mer sequences
agaccg

SEQ ID NO: 386 - DNA, 6 bp, Artificial Sequence
  oligonucleotide 6-mer sequences
taaccg

SEQ ID NO: 387 - DNA, 6 bp, Artificial Sequence
  oligonucleotide 6-mer sequences
aaaccg

SEQ ID NO: 388 - DNA, 6 bp, Artificial Sequence
  oligonucleotide 6-mer sequences
tttacg

SEQ ID NO: 389 - DNA, 6 bp, Artificial Sequence
  oligonucleotide 6-mer sequences
attacg

SEQ ID NO: 390 - DNA, 6 bp, Artificial Sequence
  oligonucleotide 6-mer sequences
tgtacg

SEQ ID NO: 391 - DNA, 6 bp, Artificial Sequence
  oligonucleotide 6-mer sequences
agtacg

SEQ ID NO: 392 - DNA, 6 bp, Artificial Sequence
  oligonucleotide 6-mer sequences
gatacg

SEQ ID NO: 393 - DNA, 6 bp, Artificial Sequence
  oligonucleotide 6-mer sequences
catacg

SEQ ID NO: 394 - DNA, 6 bp, Artificial Sequence
  oligonucleotide 6-mer sequences
ctgacg

SEQ ID NO: 395 - DNA, 6 bp, Artificial Sequence
  oligonucleotide 6-mer sequences
tcgacg

SEQ ID NO: 396 - DNA, 6 bp, Artificial Sequence
  oligonucleotide 6-mer sequences
tagacg

SEQ ID NO: 397 - DNA, 6 bp, Artificial Sequence
  oligonucleotide 6-mer sequences
gtcacg

SEQ ID NO: 398 - DNA, 6 bp, Artificial Sequence
  oligonucleotide 6-mer sequences
tacacg

SEQ ID NO: 399 - DNA, 6 bp, Artificial Sequence
  oligonucleotide 6-mer sequences
ctaacg

SEQ ID NO: 400 - DNA, 6 bp, Artificial Sequence
  oligonucleotide 6-mer sequences
acaacg

SEQ ID NO: 401 - DNA, 6 bp, Artificial Sequence
  oligonucleotide 6-mer sequences
gaaacg

SEQ ID NO: 402 - DNA, 6 bp, Artificial Sequence
  oligonucleotide 6-mer sequences
caaacg

SEQ ID NO: 403 - DNA, 6 bp, Artificial Sequence
  oligonucleotide 6-mer sequences
ttttag

SEQ ID NO: 404 - DNA, 6 bp, Artificial Sequence
  oligonucleotide 6-mer sequences
ctttag

SEQ ID NO: 405 - DNA, 6 bp, Artificial Sequence
  oligonucleotide 6-mer sequences
cgttag

SEQ ID NO: 406 - DNA, 6 bp, Artificial Sequence
  oligonucleotide 6-mer sequences
ttgtag

SEQ ID NO: 407 - DNA, 6 bp, Artificial Sequence
  oligonucleotide 6-mer sequences
ctgtag

SEQ ID NO: 408 - DNA, 6 bp, Artificial Sequence
  oligonucleotide 6-mer sequences
atgtag

SEQ ID NO: 409 - DNA, 6 bp, Artificial Sequence
  oligonucleotide 6-mer sequences
tcgtag

SEQ ID NO: 410 - DNA, 6 bp, Artificial Sequence
  oligonucleotide 6-mer sequences
ccgtag

SEQ ID NO: 411 - DNA, 6 bp, Artificial Sequence
  oligonucleotide 6-mer sequences
acgtag

SEQ ID NO: 412 - DNA, 6 bp, Artificial Sequence
  oligonucleotide 6-mer sequences
ttctag

SEQ ID NO: 413 - DNA, 6 bp, Artificial Sequence
  oligonucleotide 6-mer sequences
gtctag

SEQ ID NO: 414 - DNA, 6 bp, Artificial Sequence
  oligonucleotide 6-mer sequences
cgctag

SEQ ID NO: 415 - DNA, 6 bp, Artificial Sequence
  oligonucleotide 6-mer sequences
agctag

SEQ ID NO: 416 - DNA, 6 bp, Artificial Sequence
  oligonucleotide 6-mer sequences
gcctag

SEQ ID NO: 417 - DNA, 6 bp, Artificial Sequence
  oligonucleotide 6-mer sequences
ttatag

SEQ ID NO: 418 - DNA, 6 bp, Artificial Sequence
  oligonucleotide 6-mer sequences
gtatag

SEQ ID NO: 419 - DNA, 6 bp, Artificial Sequence
  oligonucleotide 6-mer sequences
ctatag

SEQ ID NO: 420 - DNA, 6 bp, Artificial Sequence
  oligonucleotide 6-mer sequences
atatag

SEQ ID NO: 421 - DNA, 6 bp, Artificial Sequence
  oligonucleotide 6-mer sequences
cgatag

SEQ ID NO: 422 - DNA, 6 bp, Artificial Sequence
  oligonucleotide 6-mer sequences
ccatag

SEQ ID NO: 423 - DNA, 6 bp, Artificial Sequence
  oligonucleotide 6-mer sequences
acatag

SEQ ID NO: 424 - DNA, 6 bp, Artificial Sequence
  oligonucleotide 6-mer sequences
attgag

SEQ ID NO: 425 - DNA, 6 bp, Artificial Sequence
  oligonucleotide 6-mer sequences
tgtgag

SEQ ID NO: 426 - DNA, 6 bp, Artificial Sequence
  oligonucleotide 6-mer sequences
tatgag

SEQ ID NO: 427 - DNA, 6 bp, Artificial Sequence
  oligonucleotide 6-mer sequences
catgag

SEQ ID NO: 428 - DNA, 6 bp, Artificial Sequence
  oligonucleotide 6-mer sequences
ttcgag

SEQ ID NO: 429 - DNA, 6 bp, Artificial Sequence
  oligonucleotide 6-mer sequences
ctcgag

SEQ ID NO: 430 - DNA, 6 bp, Artificial Sequence
  oligonucleotide 6-mer sequences
aacgag

SEQ ID NO: 431 - DNA, 6 bp, Artificial Sequence
  oligonucleotide 6-mer sequences
gtagag

SEQ ID NO: 432 - DNA, 6 bp, Artificial Sequence
  oligonucleotide 6-mer sequences
atagag

SEQ ID NO: 433 - DNA, 6 bp, Artificial Sequence
  oligonucleotide 6-mer sequences
tgagag

SEQ ID NO: 434 - DNA, 6 bp, Artificial Sequence
  oligonucleotide 6-mer sequences
acagag

SEQ ID NO: 435 - DNA, 6 bp, Artificial Sequence
  oligonucleotide 6-mer sequences
cttcag

SEQ ID NO: 436 - DNA, 6 bp, Artificial Sequence
  oligonucleotide 6-mer sequences
aatcag

SEQ ID NO: 437 - DNA, 6 bp, Artificial Sequence
  oligonucleotide 6-mer sequences
gcgcag

SEQ ID NO: 438 - DNA, 6 bp, Artificial Sequence
  oligonucleotide 6-mer sequences
acgcag

SEQ ID NO: 439 - DNA, 6 bp, Artificial Sequence
  oligonucleotide 6-mer sequences
taccag

SEQ ID NO: 440 - DNA, 6 bp, Artificial Sequence
  oligonucleotide 6-mer sequences
gtacag

SEQ ID NO: 441 - DNA, 6 bp, Artificial Sequence
  oligonucleotide 6-mer sequences
ctacag

SEQ ID NO: 442 - DNA, 6 bp, Artificial Sequence
  oligonucleotide 6-mer sequences
atacag

SEQ ID NO: 443 - DNA, 6 bp, Artificial Sequence
  oligonucleotide 6-mer sequences
cgacag

SEQ ID NO: 444 - DNA, 6 bp, Artificial Sequence
  oligonucleotide 6-mer sequences
gcacag

SEQ ID NO: 445 - DNA, 6 bp, Artificial Sequence
  oligonucleotide 6-mer sequences
gaacag

SEQ ID NO: 446 - DNA, 6 bp, Artificial Sequence
  oligonucleotide 6-mer sequences
gttaag

SEQ ID NO: 447 - DNA, 6 bp, Artificial Sequence
  oligonucleotide 6-mer sequences
tgtaag

SEQ ID NO: 448 - DNA, 6 bp, Artificial Sequence
  oligonucleotide 6-mer sequences
ggtaag

SEQ ID NO: 449 - DNA, 6 bp, Artificial Sequence
  oligonucleotide 6-mer sequences
cgtaag

SEQ ID NO: 450 - DNA, 6 bp, Artificial Sequence
  oligonucleotide 6-mer sequences
agtaag

SEQ ID NO: 451 - DNA, 6 bp, Artificial Sequence
  oligonucleotide 6-mer sequences
cctaag

SEQ ID NO: 452 - DNA, 6 bp, Artificial Sequence
  oligonucleotide 6-mer sequences
tataag

SEQ ID NO: 453 - DNA, 6 bp, Artificial Sequence
  oligonucleotide 6-mer sequences
gataag

SEQ ID NO: 454 - DNA, 6 bp, Artificial Sequence
  oligonucleotide 6-mer sequences
cataag

SEQ ID NO: 455 - DNA, 6 bp, Artificial Sequence
  oligonucleotide 6-mer sequences
aataag

SEQ ID NO: 456 - DNA, 6 bp, Artificial Sequence
  oligonucleotide 6-mer sequences
gtgaag

SEQ ID NO: 457 - DNA, 6 bp, Artificial Sequence
  oligonucleotide 6-mer sequences
atgaag

SEQ ID NO: 458 - DNA, 6 bp, Artificial Sequence
  oligonucleotide 6-mer sequences
tggaag

SEQ ID NO: 459 - DNA, 6 bp, Artificial Sequence
  oligonucleotide 6-mer sequences
tagaag

SEQ ID NO: 460 - DNA, 6 bp, Artificial Sequence
  oligonucleotide 6-mer sequences
gagaag

SEQ ID NO: 461 - DNA, 6 bp, Artificial Sequence
  oligonucleotide 6-mer sequences
tacaag

SEQ ID NO: 462 - DNA, 6 bp, Artificial Sequence
  oligonucleotide 6-mer sequences
aacaag

SEQ ID NO: 463 - DNA, 6 bp, Artificial Sequence
  oligonucleotide 6-mer sequences
gtaaag

SEQ ID NO: 464 - DNA, 6 bp, Artificial Sequence
  oligonucleotide 6-mer sequences
taaaag

SEQ ID NO: 465 - DNA, 6 bp, Artificial Sequence
  oligonucleotide 6-mer sequences
gttttc

SEQ ID NO: 466 - DNA, 6 bp, Artificial Sequence
  oligonucleotide 6-mer sequences
gatttc

SEQ ID NO: 467 - DNA, 6 bp, Artificial Sequence
  oligonucleotide 6-mer sequences
gagttc

SEQ ID NO: 468 - DNA, 6 bp, Artificial Sequence
  oligonucleotide 6-mer sequences
atattc

SEQ ID NO: 469 - DNA, 6 bp, Artificial Sequence
  oligonucleotide 6-mer sequences
cgattc

SEQ ID NO: 470 - DNA, 6 bp, Artificial Sequence
  oligonucleotide 6-mer sequences
agattc

SEQ ID NO: 471 - DNA, 6 bp, Artificial Sequence
  oligonucleotide 6-mer sequences
taattc

SEQ ID NO: 472 - DNA, 6 bp, Artificial Sequence
  oligonucleotide 6-mer sequences
tttgtc

SEQ ID NO: 473 - DNA, 6 bp, Artificial Sequence
  oligonucleotide 6-mer sequences
attgtc

SEQ ID NO: 474 - DNA, 6 bp, Artificial Sequence
  oligonucleotide 6-mer sequences
agtgtc

SEQ ID NO: 475 - DNA, 6 bp, Artificial Sequence
  oligonucleotide 6-mer sequences
gatgtc

SEQ ID NO: 476 - DNA, 6 bp, Artificial Sequence
  oligonucleotide 6-mer sequences
taggtc

SEQ ID NO: 477 - DNA, 6 bp, Artificial Sequence
  oligonucleotide 6-mer sequences
tacgtc

SEQ ID NO: 478 - DNA, 6 bp, Artificial Sequence
  oligonucleotide 6-mer sequences
aacgtc

SEQ ID NO: 479 - DNA, 6 bp, Artificial Sequence
  oligonucleotide 6-mer sequences
gtagtc

SEQ ID NO: 480 - DNA, 6 bp, Artificial Sequence
  oligonucleotide 6-mer sequences
ctagtc

SEQ ID NO: 481 - DNA, 6 bp, Artificial Sequence
  oligonucleotide 6-mer sequences
cgagtc

SEQ ID NO: 482 - DNA, 6 bp, Artificial Sequence
  oligonucleotide 6-mer sequences
caagtc

SEQ ID NO: 483 - DNA, 6 bp, Artificial Sequence
  oligonucleotide 6-mer sequences
aaagtc

SEQ ID NO: 484 - DNA, 6 bp, Artificial Sequence
  oligonucleotide 6-mer sequences
attctc

SEQ ID NO: 485 - DNA, 6 bp, Artificial Sequence
  oligonucleotide 6-mer sequences
cgtctc

SEQ ID NO: 486 - DNA, 6 bp, Artificial Sequence
  oligonucleotide 6-mer sequences
gctctc

SEQ ID NO: 487 - DNA, 6 bp, Artificial Sequence
  oligonucleotide 6-mer sequences
tatctc

SEQ ID NO: 488 - DNA, 6 bp, Artificial Sequence
  oligonucleotide 6-mer sequences
tgcctc

SEQ ID NO: 489 - DNA, 6 bp, Artificial Sequence
  oligonucleotide 6-mer sequences
agcctc

SEQ ID NO: 490 - DNA, 6 bp, Artificial Sequence
  oligonucleotide 6-mer sequences
gtactc

SEQ ID NO: 491 - DNA, 6 bp, Artificial Sequence
  oligonucleotide 6-mer sequences
tgactc

SEQ ID NO: 492 - DNA, 6 bp, Artificial Sequence
  oligonucleotide 6-mer sequences
taactc

SEQ ID NO: 493 - DNA, 6 bp, Artificial Sequence
  oligonucleotide 6-mer sequences
caactc

SEQ ID NO: 494 - DNA, 6 bp, Artificial Sequence
  oligonucleotide 6-mer sequences
tttatc

SEQ ID NO: 495 - DNA, 6 bp, Artificial Sequence
  oligonucleotide 6-mer sequences
attatc

SEQ ID NO: 496 - DNA, 6 bp, Artificial Sequence
  oligonucleotide 6-mer sequences
tgtatc

SEQ ID NO: 497 - DNA, 6 bp, Artificial Sequence
  oligonucleotide 6-mer sequences
agtatc

SEQ ID NO: 498 - DNA, 6 bp, Artificial Sequence
  oligonucleotide 6-mer sequences
tctatc

SEQ ID NO: 499 - DNA, 6 bp, Artificial Sequence
  oligonucleotide 6-mer sequences
gatatc

SEQ ID NO: 500 - DNA, 6 bp, Artificial Sequence
  oligonucleotide 6-mer sequences
catatc

SEQ ID NO: 501 - DNA, 6 bp, Artificial Sequence
  oligonucleotide 6-mer sequences
gtgatc

SEQ ID NO: 502 - DNA, 6 bp, Artificial Sequence
  oligonucleotide 6-mer sequences
gtcatc

SEQ ID NO: 503 - DNA, 6 bp, Artificial Sequence
  oligonucleotide 6-mer sequences
ctcatc

SEQ ID NO: 504 - DNA, 6 bp, Artificial Sequence
  oligonucleotide 6-mer sequences
tccatc

SEQ ID NO: 505 - DNA, 6 bp, Artificial Sequence
  oligonucleotide 6-mer sequences
tacatc

SEQ ID NO: 506 - DNA, 6 bp, Artificial Sequence
  oligonucleotide 6-mer sequences
cgaatc

SEQ ID NO: 507 - DNA, 6 bp, Artificial Sequence
  oligonucleotide 6-mer sequences
taaatc

SEQ ID NO: 508 - DNA, 6 bp, Artificial Sequence
  oligonucleotide 6-mer sequences
gtttgc

SEQ ID NO: 509 - DNA, 6 bp, Artificial Sequence
  oligonucleotide 6-mer sequences
tgttgc

SEQ ID NO: 510 - DNA, 6 bp, Artificial Sequence
  oligonucleotide 6-mer sequences
agttgc

SEQ ID NO: 511 - DNA, 6 bp, Artificial Sequence
  oligonucleotide 6-mer sequences
gtgtgc

SEQ ID NO: 512 - DNA, 6 bp, Artificial Sequence
  oligonucleotide 6-mer sequences
ctgtgc

SEQ ID NO: 513 - DNA, 6 bp, Artificial Sequence
  oligonucleotide 6-mer sequences
atgtgc

SEQ ID NO: 514 - DNA, 6 bp, Artificial Sequence
  oligonucleotide 6-mer sequences
tagtgc

SEQ ID NO: 515 - DNA, 6 bp, Artificial Sequence
  oligonucleotide 6-mer sequences
tactgc

SEQ ID NO: 516 - DNA, 6 bp, Artificial Sequence
  oligonucleotide 6-mer sequences
ttatgc

SEQ ID NO: 517 - DNA, 6 bp, Artificial Sequence
  oligonucleotide 6-mer sequences
gtatgc

SEQ ID NO: 518 - DNA, 6 bp, Artificial Sequence
  oligonucleotide 6-mer sequences
ctatgc

SEQ ID NO: 519 - DNA, 6 bp, Artificial Sequence
  oligonucleotide 6-mer sequences
tgatgc

SEQ ID NO: 520 - DNA, 6 bp, Artificial Sequence
  oligonucleotide 6-mer sequences
ggatgc

SEQ ID NO: 521 - DNA, 6 bp, Artificial Sequence
  oligonucleotide 6-mer sequences
taatgc

SEQ ID NO: 522 - DNA, 6 bp, Artificial Sequence
  oligonucleotide 6-mer sequences
caatgc

SEQ ID NO: 523 - DNA, 6 bp, Artificial Sequence
  oligonucleotide 6-mer sequences
gttggc

SEQ ID NO: 524 - DNA, 6 bp, Artificial Sequence
  oligonucleotide 6-mer sequences
aatggc

SEQ ID NO: 525 - DNA, 6 bp, Artificial Sequence
  oligonucleotide 6-mer sequences
taaggc

SEQ ID NO: 526 - DNA, 6 bp, Artificial Sequence
  oligonucleotide 6-mer sequences
gaaggc

SEQ ID NO: 527 - DNA, 6 bp, Artificial Sequence
  oligonucleotide 6-mer sequences
tgtcgc

SEQ ID NO: 528 - DNA, 6 bp, Artificial Sequence
  oligonucleotide 6-mer sequences
agtcgc

SEQ ID NO: 529 - DNA, 6 bp, Artificial Sequence
  oligonucleotide 6-mer sequences
tatcgc

SEQ ID NO: 530 - DNA, 6 bp, Artificial Sequence
  oligonucleotide 6-mer sequences
aagcgc

SEQ ID NO: 531 - DNA, 6 bp, Artificial Sequence
  oligonucleotide 6-mer sequences
ttacgc

SEQ ID NO: 532 - DNA, 6 bp, Artificial Sequence
  oligonucleotide 6-mer sequences
ctacgc

SEQ ID NO: 533 - DNA, 6 bp, Artificial Sequence
  oligonucleotide 6-mer sequences
tgacgc

SEQ ID NO: 534 - DNA, 6 bp, Artificial Sequence
  oligonucleotide 6-mer sequences
gctagc

SEQ ID NO: 535 - DNA, 6 bp, Artificial Sequence
  oligonucleotide 6-mer sequences
actagc

SEQ ID NO: 536 - DNA, 6 bp, Artificial Sequence
  oligonucleotide 6-mer sequences
tatagc

SEQ ID NO: 537 - DNA, 6 bp, Artificial Sequence
  oligonucleotide 6-mer sequences
gatagc

SEQ ID NO: 538 - DNA, 6 bp, Artificial Sequence
  oligonucleotide 6-mer sequences
catagc

SEQ ID NO: 539 - DNA, 6 bp, Artificial Sequence
  oligonucleotide 6-mer sequences
aatagc

SEQ ID NO: 540 - DNA, 6 bp, Artificial Sequence
  oligonucleotide 6-mer sequences
tcgagc

SEQ ID NO: 541 - DNA, 6 bp, Artificial Sequence
  oligonucleotide 6-mer sequences
atcagc

SEQ ID NO: 542 - DNA, 6 bp, Artificial Sequence
  oligonucleotide 6-mer sequences
tacagc

SEQ ID NO: 543 - DNA, 6 bp, Artificial Sequence
  oligonucleotide 6-mer sequences
cacagc

SEQ ID NO: 544 - DNA, 6 bp, Artificial Sequence
  oligonucleotide 6-mer sequences
gtaagc

SEQ ID NO: 545 - DNA, 6 bp, Artificial Sequence
  oligonucleotide 6-mer sequences
ctaagc

SEQ ID NO: 546 - DNA, 6 bp, Artificial Sequence
  oligonucleotide 6-mer sequences
ataagc

SEQ ID NO: 547 - DNA, 6 bp, Artificial Sequence
  oligonucleotide 6-mer sequences
tgaagc

SEQ ID NO: 548 - DNA, 6 bp, Artificial Sequence
  oligonucleotide 6-mer sequences
gcttcc

SEQ ID NO: 549 - DNA, 6 bp, Artificial Sequence
  oligonucleotide 6-mer sequences
aggtcc

SEQ ID NO: 550 - DNA, 6 bp, Artificial Sequence
  oligonucleotide 6-mer sequences
acgtcc

SEQ ID NO: 551 - DNA, 6 bp, Artificial Sequence
  oligonucleotide 6-mer sequences
tagtcc

SEQ ID NO: 552 - DNA, 6 bp, Artificial Sequence
  oligonucleotide 6-mer sequences
aagtcc

SEQ ID NO: 553 - DNA, 6 bp, Artificial Sequence
  oligonucleotide 6-mer sequences
gtctcc

SEQ ID NO: 554 - DNA, 6 bp, Artificial Sequence
  oligonucleotide 6-mer sequences
agctcc

SEQ ID NO: 555 - DNA, 6 bp, Artificial Sequence
  oligonucleotide 6-mer sequences
aaatcc

SEQ ID NO: 556 - DNA, 6 bp, Artificial Sequence
  oligonucleotide 6-mer sequences
tgtgcc

SEQ ID NO: 557 - DNA, 6 bp, Artificial Sequence
  oligonucleotide 6-mer sequences
tatgcc

SEQ ID NO: 558 - DNA, 6 bp, Artificial Sequence
  oligonucleotide 6-mer sequences
gatgcc

SEQ ID NO: 559 - DNA, 6 bp, Artificial Sequence
  oligonucleotide 6-mer sequences
taggcc

SEQ ID NO: 560 - DNA, 6 bp, Artificial Sequence
  oligonucleotide 6-mer sequences
ctagcc

SEQ ID NO: 561 - DNA, 6 bp, Artificial Sequence
  oligonucleotide 6-mer sequences
atagcc

SEQ ID NO: 562 - DNA, 6 bp, Artificial Sequence
  oligonucleotide 6-mer sequences
acagcc

SEQ ID NO: 563 - DNA, 6 bp, Artificial Sequence
  oligonucleotide 6-mer sequences
agtccc

SEQ ID NO: 564 - DNA, 6 bp, Artificial Sequence
  oligonucleotide 6-mer sequences
tagccc

SEQ ID NO: 565 - DNA, 6 bp, Artificial Sequence
  oligonucleotide 6-mer sequences
gtaccc

SEQ ID NO: 566 - DNA, 6 bp, Artificial Sequence
  oligonucleotide 6-mer sequences
ctaccc

SEQ ID NO: 567 - DNA, 6 bp, Artificial Sequence
  oligonucleotide 6-mer sequences
ggtacc

SEQ ID NO: 568 - DNA, 6 bp, Artificial Sequence
  oligonucleotide 6-mer sequences
cgtacc

SEQ ID NO: 569 - DNA, 6 bp, Artificial Sequence
  oligonucleotide 6-mer sequences
agtacc

SEQ ID NO: 570 - DNA, 6 bp, Artificial Sequence
  oligonucleotide 6-mer sequences
tctacc

SEQ ID NO: 571 - DNA, 6 bp, Artificial Sequence
  oligonucleotide 6-mer sequences
cctacc

SEQ ID NO: 572 - DNA, 6 bp, Artificial Sequence
  oligonucleotide 6-mer sequences
gatacc

SEQ ID NO: 573 - DNA, 6 bp, Artificial Sequence
  oligonucleotide 6-mer sequences
aatacc

SEQ ID NO: 574 - DNA, 6 bp, Artificial Sequence
  oligonucleotide 6-mer sequences
gtgacc

SEQ ID NO: 575 - DNA, 6 bp, Artificial Sequence
  oligonucleotide 6-mer sequences
tggacc

SEQ ID NO: 576 - DNA, 6 bp, Artificial Sequence
  oligonucleotide 6-mer sequences
atcacc

SEQ ID NO: 577 - DNA, 6 bp, Artificial Sequence
  oligonucleotide 6-mer sequences
gtaacc

SEQ ID NO: 578 - DNA, 6 bp, Artificial Sequence
  oligonucleotide 6-mer sequences
tattac

SEQ ID NO: 579 - DNA, 6 bp, Artificial Sequence
  oligonucleotide 6-mer sequences
cattac

SEQ ID NO: 580 - DNA, 6 bp, Artificial Sequence
  oligonucleotide 6-mer sequences
ttgtac

SEQ ID NO: 581 - DNA, 6 bp, Artificial Sequence
  oligonucleotide 6-mer sequences
atgtac

SEQ ID NO: 582 - DNA, 6 bp, Artificial Sequence
  oligonucleotide 6-mer sequences
aggtac

SEQ ID NO: 583 - DNA, 6 bp, Artificial Sequence
  oligonucleotide 6-mer sequences
tagtac

SEQ ID NO: 584 - DNA, 6 bp, Artificial Sequence
  oligonucleotide 6-mer sequences
gagtac

SEQ ID NO: 585 - DNA, 6 bp, Artificial Sequence
  oligonucleotide 6-mer sequences
cagtac

SEQ ID NO: 586 - DNA, 6 bp, Artificial Sequence
  oligonucleotide 6-mer sequences
aagtac

SEQ ID NO: 587 - DNA, 6 bp, Artificial Sequence
  oligonucleotide 6-mer sequences
ctctac

SEQ ID NO: 588 - DNA, 6 bp, Artificial Sequence
  oligonucleotide 6-mer sequences
atctac

SEQ ID NO: 589 - DNA, 6 bp, Artificial Sequence
  oligonucleotide 6-mer sequences
gcctac

SEQ ID NO: 590 - DNA, 6 bp, Artificial Sequence
  oligonucleotide 6-mer sequences
ccctac

SEQ ID NO: 591 - DNA, 6 bp, Artificial Sequence
  oligonucleotide 6-mer sequences
ttatac

SEQ ID NO: 592 - DNA, 6 bp, Artificial Sequence
  oligonucleotide 6-mer sequences
gtatac

SEQ ID NO: 593 - DNA, 6 bp, Artificial Sequence
  oligonucleotide 6-mer sequences
ggatac

SEQ ID NO: 594 - DNA, 6 bp, Artificial Sequence
  oligonucleotide 6-mer sequences
cgatac

SEQ ID NO: 595 - DNA, 6 bp, Artificial Sequence
  oligonucleotide 6-mer sequences
agatac

SEQ ID NO: 596 - DNA, 6 bp, Artificial Sequence
  oligonucleotide 6-mer sequences
tcatac

SEQ ID NO: 597 - DNA, 6 bp, Artificial Sequence
  oligonucleotide 6-mer sequences
gcatac

SEQ ID NO: 598 - DNA, 6 bp, Artificial Sequence
  oligonucleotide 6-mer sequences
taatac

SEQ ID NO: 599 - DNA, 6 bp, Artificial Sequence
  oligonucleotide 6-mer sequences
gaatac

SEQ ID NO: 600 - DNA, 6 bp, Artificial Sequence
  oligonucleotide 6-mer sequences
aaatac

SEQ ID NO: 601 - DNA, 6 bp, Artificial Sequence
  oligonucleotide 6-mer sequences
tgtgac

SEQ ID NO: 602 - DNA, 6 bp, Artificial Sequence
  oligonucleotide 6-mer sequences
ggtgac

SEQ ID NO: 603 - DNA, 6 bp, Artificial Sequence
  oligonucleotide 6-mer sequences
agtgac

SEQ ID NO: 604 - DNA, 6 bp, Artificial Sequence
  oligonucleotide 6-mer sequences
cctgac

SEQ ID NO: 605 - DNA, 6 bp, Artificial Sequence
  oligonucleotide 6-mer sequences
catgac

SEQ ID NO: 606 - DNA, 6 bp, Artificial Sequence
  oligonucleotide 6-mer sequences
atggac

SEQ ID NO: 607 - DNA, 6 bp, Artificial Sequence
  oligonucleotide 6-mer sequences
tgggac

SEQ ID NO: 608 - DNA, 6 bp, Artificial Sequence
  oligonucleotide 6-mer sequences
gtcgac

SEQ ID NO: 609 - DNA, 6 bp, Artificial Sequence
  oligonucleotide 6-mer sequences
atcgac

SEQ ID NO: 610 - DNA, 6 bp, Artificial Sequence
  oligonucleotide 6-mer sequences
tgcgac

SEQ ID NO: 611 - DNA, 6 bp, Artificial Sequence
  oligonucleotide 6-mer sequences
aacgac

SEQ ID NO: 612 - DNA, 6 bp, Artificial Sequence
  oligonucleotide 6-mer sequences
ctagac

SEQ ID NO: 613 - DNA, 6 bp, Artificial Sequence
  oligonucleotide 6-mer sequences
atagac

SEQ ID NO: 614 - DNA, 6 bp, Artificial Sequence
  oligonucleotide 6-mer sequences
agagac

SEQ ID NO: 615 - DNA, 6 bp, Artificial Sequence
  oligonucleotide 6-mer sequences
taagac

SEQ ID NO: 616 - DNA, 6 bp, Artificial Sequence
  oligonucleotide 6-mer sequences
actcac

SEQ ID NO: 617 - DNA, 6 bp, Artificial Sequence
  oligonucleotide 6-mer sequences
tatcac

SEQ ID NO: 618 - DNA, 6 bp, Artificial Sequence
  oligonucleotide 6-mer sequences
gatcac

SEQ ID NO: 619 - DNA, 6 bp, Artificial Sequence
  oligonucleotide 6-mer sequences
tcgcac

SEQ ID NO: 620 - DNA, 6 bp, Artificial Sequence
  oligonucleotide 6-mer sequences
tgccac

SEQ ID NO: 621 - DNA, 6 bp, Artificial Sequence
  oligonucleotide 6-mer sequences
aaccac

SEQ ID NO: 622 - DNA, 6 bp, Artificial Sequence
  oligonucleotide 6-mer sequences
gtacac

SEQ ID NO: 623 - DNA, 6 bp, Artificial Sequence
  oligonucleotide 6-mer sequences
agacac

SEQ ID NO: 624 - DNA, 6 bp, Artificial Sequence
  oligonucleotide 6-mer sequences
aaacac

SEQ ID NO: 625 - DNA, 6 bp, Artificial Sequence
  oligonucleotide 6-mer sequences
gttaac

SEQ ID NO: 626 - DNA, 6 bp, Artificial Sequence
  oligonucleotide 6-mer sequences
tctaac

SEQ ID NO: 627 - DNA, 6 bp, Artificial Sequence
  oligonucleotide 6-mer sequences
gctaac

SEQ ID NO: 628 - DNA, 6 bp, Artificial Sequence
  oligonucleotide 6-mer sequences
tataac

SEQ ID NO: 629 - DNA, 6 bp, Artificial Sequence
  oligonucleotide 6-mer sequences
ccgaac

SEQ ID NO: 630 - DNA, 6 bp, Artificial Sequence
  oligonucleotide 6-mer sequences
cagaac

SEQ ID NO: 631 - DNA, 6 bp, Artificial Sequence
  oligonucleotide 6-mer sequences
cgcaac

SEQ ID NO: 632 - DNA, 6 bp, Artificial Sequence
  oligonucleotide 6-mer sequences
cccaac

SEQ ID NO: 633 - DNA, 6 bp, Artificial Sequence
  oligonucleotide 6-mer sequences
tacaac

SEQ ID NO: 634 - DNA, 6 bp, Artificial Sequence
  oligonucleotide 6-mer sequences
cacaac

SEQ ID NO: 635 - DNA, 6 bp, Artificial Sequence
  oligonucleotide 6-mer sequences
ataaac

SEQ ID NO: 636 - DNA, 6 bp, Artificial Sequence
  oligonucleotide 6-mer sequences
tgaaac

SEQ ID NO: 637 - DNA, 6 bp, Artificial Sequence
  oligonucleotide 6-mer sequences
agaaac

SEQ ID NO: 638 - DNA, 6 bp, Artificial Sequence
  oligonucleotide 6-mer sequences
gcaaac

SEQ ID NO: 639 - DNA, 6 bp, Artificial Sequence
  oligonucleotide 6-mer sequences
ccaaac

SEQ ID NO: 640 - DNA, 6 bp, Artificial Sequence
  oligonucleotide 6-mer sequences
atttta

SEQ ID NO: 641 - DNA, 6 bp, Artificial Sequence
  oligonucleotide 6-mer sequences
tgttta

SEQ ID NO: 642 - DNA, 6 bp, Artificial Sequence
  oligonucleotide 6-mer sequences
aattta

SEQ ID NO: 643 - DNA, 6 bp, Artificial Sequence
  oligonucleotide 6-mer sequences
atgtta

SEQ ID NO: 644 - DNA, 6 bp, Artificial Sequence
  oligonucleotide 6-mer sequences
cggtta

SEQ ID NO: 645 - DNA, 6 bp, Artificial Sequence
  oligonucleotide 6-mer sequences
acgtta

SEQ ID NO: 646 - DNA, 6 bp, Artificial Sequence
  oligonucleotide 6-mer sequences
gagtta

SEQ ID NO: 647 - DNA, 6 bp, Artificial Sequence
  oligonucleotide 6-mer sequences
gtctta

SEQ ID NO: 648 - DNA, 6 bp, Artificial Sequence
  oligonucleotide 6-mer sequences
atctta

SEQ ID NO: 649 - DNA, 6 bp, Artificial Sequence
  oligonucleotide 6-mer sequences
cgctta

SEQ ID NO: 650 - DNA, 6 bp, Artificial Sequence
  oligonucleotide 6-mer sequences
aactta

SEQ ID NO: 651 - DNA, 6 bp, Artificial Sequence
  oligonucleotide 6-mer sequences
atatta

SEQ ID NO: 652 - DNA, 6 bp, Artificial Sequence
  oligonucleotide 6-mer sequences
agatta

SEQ ID NO: 653 - DNA, 6 bp, Artificial Sequence
  oligonucleotide 6-mer sequences
tcatta

SEQ ID NO: 654 - DNA, 6 bp, Artificial Sequence
  oligonucleotide 6-mer sequences
acatta

SEQ ID NO: 655 - DNA, 6 bp, Artificial Sequence
  oligonucleotide 6-mer sequences
gttgta

SEQ ID NO: 656 - DNA, 6 bp, Artificial Sequence
  oligonucleotide 6-mer sequences
cttgta

SEQ ID NO: 657 - DNA, 6 bp, Artificial Sequence
  oligonucleotide 6-mer sequences
attgta

SEQ ID NO: 658 - DNA, 6 bp, Artificial Sequence
  oligonucleotide 6-mer sequences
cgtgta

SEQ ID NO: 659 - DNA, 6 bp, Artificial Sequence
  oligonucleotide 6-mer sequences
tatgta

SEQ ID NO: 660 - DNA, 6 bp, Artificial Sequence
  oligonucleotide 6-mer sequences
gatgta

SEQ ID NO: 661 - DNA, 6 bp, Artificial Sequence
  oligonucleotide 6-mer sequences
aatgta

SEQ ID NO: 662 - DNA, 6 bp, Artificial Sequence
  oligonucleotide 6-mer sequences
ttggta

SEQ ID NO: 663 - DNA, 6 bp, Artificial Sequence
  oligonucleotide 6-mer sequences
ctggta

SEQ ID NO: 664 - DNA, 6 bp, Artificial Sequence
  oligonucleotide 6-mer sequences
gaggta

SEQ ID NO: 665 - DNA, 6 bp, Artificial Sequence
  oligonucleotide 6-mer sequences
aaggta

SEQ ID NO: 666 - DNA, 6 bp, Artificial Sequence
  oligonucleotide 6-mer sequences
gtcgta

SEQ ID NO: 667 - DNA, 6 bp, Artificial Sequence
  oligonucleotide 6-mer sequences
atcgta

SEQ ID NO: 668 - DNA, 6 bp, Artificial Sequence
  oligonucleotide 6-mer sequences
agcgta

SEQ ID NO: 669 - DNA, 6 bp, Artificial Sequence
  oligonucleotide 6-mer sequences
cccgta

SEQ ID NO: 670 - DNA, 6 bp, Artificial Sequence
  oligonucleotide 6-mer sequences
accgta

SEQ ID NO: 671 - DNA, 6 bp, Artificial Sequence
  oligonucleotide 6-mer sequences
gacgta

SEQ ID NO: 672 - DNA, 6 bp, Artificial Sequence
  oligonucleotide 6-mer sequences
cacgta

SEQ ID NO: 673 - DNA, 6 bp, Artificial Sequence
  oligonucleotide 6-mer sequences
aacgta

SEQ ID NO: 674 - DNA, 6 bp, Artificial Sequence
  oligonucleotide 6-mer sequences
ctagta

SEQ ID NO: 675 - DNA, 6 bp, Artificial Sequence
  oligonucleotide 6-mer sequences
tgagta

SEQ ID NO: 676 - DNA, 6 bp, Artificial Sequence
  oligonucleotide 6-mer sequences
ggagta

SEQ ID NO: 677 - DNA, 6 bp, Artificial Sequence
  oligonucleotide 6-mer sequences
cgagta

SEQ ID NO: 678 - DNA, 6 bp, Artificial Sequence
  oligonucleotide 6-mer sequences
acagta

SEQ ID NO: 679 - DNA, 6 bp, Artificial Sequence
  oligonucleotide 6-mer sequences
taagta

SEQ ID NO: 680 - DNA, 6 bp, Artificial Sequence
  oligonucleotide 6-mer sequences
tttcta

SEQ ID NO: 681 - DNA, 6 bp, Artificial Sequence
  oligonucleotide 6-mer sequences
cttcta

SEQ ID NO: 682 - DNA, 6 bp, Artificial Sequence
  oligonucleotide 6-mer sequences
agtcta

SEQ ID NO: 683 - DNA, 6 bp, Artificial Sequence
  oligonucleotide 6-mer sequences
tctcta

SEQ ID NO: 684 - DNA, 6 bp, Artificial Sequence
  oligonucleotide 6-mer sequences
gatcta

SEQ ID NO: 685 - DNA, 6 bp, Artificial Sequence
  oligonucleotide 6-mer sequences
gtgcta

SEQ ID NO: 686 - DNA, 6 bp, Artificial Sequence
  oligonucleotide 6-mer sequences
aggcta

SEQ ID NO: 687 - DNA, 6 bp, Artificial Sequence
  oligonucleotide 6-mer sequences
gcgcta

SEQ ID NO: 688 - DNA, 6 bp, Artificial Sequence
  oligonucleotide 6-mer sequences
tagcta

SEQ ID NO: 689 - DNA, 6 bp, Artificial Sequence
  oligonucleotide 6-mer sequences
aagcta

SEQ ID NO: 690 - DNA, 6 bp, Artificial Sequence
  oligonucleotide 6-mer sequences
atccta

SEQ ID NO: 691 - DNA, 6 bp, Artificial Sequence
  oligonucleotide 6-mer sequences
tgccta

SEQ ID NO: 692 - DNA, 6 bp, Artificial Sequence
  oligonucleotide 6-mer sequences
ggccta

SEQ ID NO: 693 - DNA, 6 bp, Artificial Sequence
  oligonucleotide 6-mer sequences
cgccta

SEQ ID NO: 694 - DNA, 6 bp, Artificial Sequence
  oligonucleotide 6-mer sequences
acccta

SEQ ID NO: 695 - DNA, 6 bp, Artificial Sequence
  oligonucleotide 6-mer sequences
gaccta

SEQ ID NO: 696 - DNA, 6 bp, Artificial Sequence
  oligonucleotide 6-mer sequences
aaccta

SEQ ID NO: 697 - DNA, 6 bp, Artificial Sequence
  oligonucleotide 6-mer sequences
ttacta

SEQ ID NO: 698 - DNA, 6 bp, Artificial Sequence
  oligonucleotide 6-mer sequences
gtacta

SEQ ID NO: 699 - DNA, 6 bp, Artificial Sequence
  oligonucleotide 6-mer sequences
atacta

SEQ ID NO: 700 - DNA, 6 bp, Artificial Sequence
  oligonucleotide 6-mer sequences
gcacta

SEQ ID NO: 701 - DNA, 6 bp, Artificial Sequence
  oligonucleotide 6-mer sequences
acacta

SEQ ID NO: 702 - DNA, 6 bp, Artificial Sequence
  oligonucleotide 6-mer sequences
tttata

SEQ ID NO: 703 - DNA, 6 bp, Artificial Sequence
  oligonucleotide 6-mer sequences
gttata

SEQ ID NO: 704 - DNA, 6 bp, Artificial Sequence
  oligonucleotide 6-mer sequences
cttata

SEQ ID NO: 705 - DNA, 6 bp, Artificial Sequence
  oligonucleotide 6-mer sequences
attata

SEQ ID NO: 706 - DNA, 6 bp, Artificial Sequence
  oligonucleotide 6-mer sequences
tgtata

SEQ ID NO: 707 - DNA, 6 bp, Artificial Sequence
  oligonucleotide 6-mer sequences
ggtata

SEQ ID NO: 708 - DNA, 6 bp, Artificial Sequence
  oligonucleotide 6-mer sequences
cgtata

SEQ ID NO: 709 - DNA, 6 bp, Artificial Sequence
  oligonucleotide 6-mer sequences
agtata

SEQ ID NO: 710 - DNA, 6 bp, Artificial Sequence
  oligonucleotide 6-mer sequences
gctata

SEQ ID NO: 711 - DNA, 6 bp, Artificial Sequence
  oligonucleotide 6-mer sequences
actata

SEQ ID NO: 712 - DNA, 6 bp, Artificial Sequence
  oligonucleotide 6-mer sequences
tatata

SEQ ID NO: 713 - DNA, 6 bp, Artificial Sequence
  oligonucleotide 6-mer sequences
gatata

SEQ ID NO: 714 - DNA, 6 bp, Artificial Sequence
  oligonucleotide 6-mer sequences
catata

SEQ ID NO: 715 - DNA, 6 bp, Artificial Sequence
  oligonucleotide 6-mer sequences
gggata

SEQ ID NO: 716 - DNA, 6 bp, Artificial Sequence
  oligonucleotide 6-mer sequences
aggata

SEQ ID NO: 717 - DNA, 6 bp, Artificial Sequence
  oligonucleotide 6-mer sequences
tcgata

SEQ ID NO: 718 - DNA, 6 bp, Artificial Sequence
  oligonucleotide 6-mer sequences
gcgata

SEQ ID NO: 719 - DNA, 6 bp, Artificial Sequence
  oligonucleotide 6-mer sequences
ccgata

SEQ ID NO: 720 - DNA, 6 bp, Artificial Sequence
  oligonucleotide 6-mer sequences
acgata

SEQ ID NO: 721 - DNA, 6 bp, Artificial Sequence
  oligonucleotide 6-mer sequences
gagata

SEQ ID NO: 722 - DNA, 6 bp, Artificial Sequence
  oligonucleotide 6-mer sequences
cagata

SEQ ID NO: 723 - DNA, 6 bp, Artificial Sequence
  oligonucleotide 6-mer sequences
aagata

SEQ ID NO: 724 - DNA, 6 bp, Artificial Sequence
  oligonucleotide 6-mer sequences
ctcata

SEQ ID NO: 725 - DNA, 6 bp, Artificial Sequence
  oligonucleotide 6-mer sequences
atcata

SEQ ID NO: 726 - DNA, 6 bp, Artificial Sequence
  oligonucleotide 6-mer sequences
tgcata

SEQ ID NO: 727 - DNA, 6 bp, Artificial Sequence
  oligonucleotide 6-mer sequences
cgcata

SEQ ID NO: 728 - DNA, 6 bp, Artificial Sequence
  oligonucleotide 6-mer sequences
tccata

SEQ ID NO: 729 - DNA, 6 bp, Artificial Sequence
  oligonucleotide 6-mer sequences
tacata

SEQ ID NO: 730 - DNA, 6 bp, Artificial Sequence
  oligonucleotide 6-mer sequences
gacata

SEQ ID NO: 731 - DNA, 6 bp, Artificial Sequence
  oligonucleotide 6-mer sequences
aacata

SEQ ID NO: 732 - DNA, 6 bp, Artificial Sequence
  oligonucleotide 6-mer sequences
gtaata

SEQ ID NO: 733 - DNA, 6 bp, Artificial Sequence
  oligonucleotide 6-mer sequences
cgaata

SEQ ID NO: 734 - DNA, 6 bp, Artificial Sequence
  oligonucleotide 6-mer sequences
ccaata

SEQ ID NO: 735 - DNA, 6 bp, Artificial Sequence
  oligonucleotide 6-mer sequences
acaata

SEQ ID NO: 736 - DNA, 6 bp, Artificial Sequence
  oligonucleotide 6-mer sequences
taaata

SEQ ID NO: 737 - DNA, 6 bp, Artificial Sequence
  oligonucleotide 6-mer sequences
caaata

SEQ ID NO: 738 - DNA, 6 bp, Artificial Sequence
  oligonucleotide 6-mer sequences
cgttga

SEQ ID NO: 739 - DNA, 6 bp, Artificial Sequence
  oligonucleotide 6-mer sequences
acttga

SEQ ID NO: 740 - DNA, 6 bp, Artificial Sequence
  oligonucleotide 6-mer sequences
gattga

SEQ ID NO: 741 - DNA, 6 bp, Artificial Sequence
  oligonucleotide 6-mer sequences
aattga

SEQ ID NO: 742 - DNA, 6 bp, Artificial Sequence
  oligonucleotide 6-mer sequences
gtgtga

SEQ ID NO: 743 - DNA, 6 bp, Artificial Sequence
  oligonucleotide 6-mer sequences
atgtga

SEQ ID NO: 744 - DNA, 6 bp, Artificial Sequence
  oligonucleotide 6-mer sequences
tggtga

SEQ ID NO: 745 - DNA, 6 bp, Artificial Sequence
  oligonucleotide 6-mer sequences
cggtga

SEQ ID NO: 746 - DNA, 6 bp, Artificial Sequence
  oligonucleotide 6-mer sequences
ccgtga

SEQ ID NO: 747 - DNA, 6 bp, Artificial Sequence
  oligonucleotide 6-mer sequences
acgtga

SEQ ID NO: 748 - DNA, 6 bp, Artificial Sequence
  oligonucleotide 6-mer sequences
gagtga

SEQ ID NO: 749 - DNA, 6 bp, Artificial Sequence
  oligonucleotide 6-mer sequences
tgctga

SEQ ID NO: 750 - DNA, 6 bp, Artificial Sequence
  oligonucleotide 6-mer sequences
acctga

SEQ ID NO: 751 - DNA, 6 bp, Artificial Sequence
  oligonucleotide 6-mer sequences
cactga

SEQ ID NO: 752 - DNA, 6 bp, Artificial Sequence
  oligonucleotide 6-mer sequences
gtatga

SEQ ID NO: 753 - DNA, 6 bp, Artificial Sequence
  oligonucleotide 6-mer sequences
atatga

SEQ ID NO: 754 - DNA, 6 bp, Artificial Sequence
  oligonucleotide 6-mer sequences
ggatga

SEQ ID NO: 755 - DNA, 6 bp, Artificial Sequence
  oligonucleotide 6-mer sequences
cgatga

SEQ ID NO: 756 - DNA, 6 bp, Artificial Sequence
  oligonucleotide 6-mer sequences
tcatga

SEQ ID NO: 757 - DNA, 6 bp, Artificial Sequence
  oligonucleotide 6-mer sequences
gcatga

SEQ ID NO: 758 - DNA, 6 bp, Artificial Sequence
  oligonucleotide 6-mer sequences
acatga

SEQ ID NO: 759 - DNA, 6 bp, Artificial Sequence
  oligonucleotide 6-mer sequences
gaatga

SEQ ID NO: 760 - DNA, 6 bp, Artificial Sequence
  oligonucleotide 6-mer sequences
tgtgga

SEQ ID NO: 761 - DNA, 6 bp, Artificial Sequence
  oligonucleotide 6-mer sequences
cctgga

SEQ ID NO: 762 - DNA, 6 bp, Artificial Sequence
  oligonucleotide 6-mer sequences
tatgga

SEQ ID NO: 763 - DNA, 6 bp, Artificial Sequence
  oligonucleotide 6-mer sequences
ctggga

SEQ ID NO: 764 - DNA, 6 bp, Artificial Sequence
  oligonucleotide 6-mer sequences
ctcgga

SEQ ID NO: 765 - DNA, 6 bp, Artificial Sequence
  oligonucleotide 6-mer sequences
tgagga

SEQ ID NO: 766 - DNA, 6 bp, Artificial Sequence
  oligonucleotide 6-mer sequences
taagga

SEQ ID NO: 767 - DNA, 6 bp, Artificial Sequence
  oligonucleotide 6-mer sequences
attcga

SEQ ID NO: 768 - DNA, 6 bp, Artificial Sequence
  oligonucleotide 6-mer sequences
cgtcga

SEQ ID NO: 769 - DNA, 6 bp, Artificial Sequence
  oligonucleotide 6-mer sequences
agtcga

SEQ ID NO: 770 - DNA, 6 bp, Artificial Sequence
  oligonucleotide 6-mer sequences
gctcga

SEQ ID NO: 771 - DNA, 6 bp, Artificial Sequence
  oligonucleotide 6-mer sequences
actcga

SEQ ID NO: 772 - DNA, 6 bp, Artificial Sequence
  oligonucleotide 6-mer sequences
tatcga

SEQ ID NO: 773 - DNA, 6 bp, Artificial Sequence
  oligonucleotide 6-mer sequences
gatcga

SEQ ID NO: 774 - DNA, 6 bp, Artificial Sequence
  oligonucleotide 6-mer sequences
ttgcga

SEQ ID NO: 775 - DNA, 6 bp, Artificial Sequence
  oligonucleotide 6-mer sequences
atgcga

SEQ ID NO: 776 - DNA, 6 bp, Artificial Sequence
  oligonucleotide 6-mer sequences
acgcga

SEQ ID NO: 777 - DNA, 6 bp, Artificial Sequence
  oligonucleotide 6-mer sequences
gtccga

SEQ ID NO: 778 - DNA, 6 bp, Artificial Sequence
  oligonucleotide 6-mer sequences
taccga

SEQ ID NO: 779 - DNA, 6 bp, Artificial Sequence
  oligonucleotide 6-mer sequences
cgacga

SEQ ID NO: 780 - DNA, 6 bp, Artificial Sequence
  oligonucleotide 6-mer sequences
agacga

SEQ ID NO: 781 - DNA, 6 bp, Artificial Sequence
  oligonucleotide 6-mer sequences
gcacga

SEQ ID NO: 782 - DNA, 6 bp, Artificial Sequence
  oligonucleotide 6-mer sequences
acacga

SEQ ID NO: 783 - DNA, 6 bp, Artificial Sequence
  oligonucleotide 6-mer sequences
taacga

SEQ ID NO: 784 - DNA, 6 bp, Artificial Sequence
  oligonucleotide 6-mer sequences
gaacga

SEQ ID NO: 785 - DNA, 6 bp, Artificial Sequence
  oligonucleotide 6-mer sequences
caacga

SEQ ID NO: 786 - DNA, 6 bp, Artificial Sequence
  oligonucleotide 6-mer sequences
tttaga

SEQ ID NO: 787 - DNA, 6 bp, Artificial Sequence
  oligonucleotide 6-mer sequences
tgtaga

SEQ ID NO: 788 - DNA, 6 bp, Artificial Sequence
  oligonucleotide 6-mer sequences
cgtaga

SEQ ID NO: 789 - DNA, 6 bp, Artificial Sequence
  oligonucleotide 6-mer sequences
agtaga

SEQ ID NO: 790 - DNA, 6 bp, Artificial Sequence
  oligonucleotide 6-mer sequences
gctaga

SEQ ID NO: 791 - DNA, 6 bp, Artificial Sequence
  oligonucleotide 6-mer sequences
cctaga

SEQ ID NO: 792 - DNA, 6 bp, Artificial Sequence
  oligonucleotide 6-mer sequences
tataga

SEQ ID NO: 793 - DNA, 6 bp, Artificial Sequence
  oligonucleotide 6-mer sequences
gataga

SEQ ID NO: 794 - DNA, 6 bp, Artificial Sequence
  oligonucleotide 6-mer sequences
cataga

SEQ ID NO: 795 - DNA, 6 bp, Artificial Sequence
  oligonucleotide 6-mer sequences
gtgaga

SEQ ID NO: 796 - DNA, 6 bp, Artificial Sequence
  oligonucleotide 6-mer sequences
atgaga

SEQ ID NO: 797 - DNA, 6 bp, Artificial Sequence
  oligonucleotide 6-mer sequences
acgaga

SEQ ID NO: 798 - DNA, 6 bp, Artificial Sequence
  oligonucleotide 6-mer sequences
tagaga

SEQ ID NO: 799 - DNA, 6 bp, Artificial Sequence
  oligonucleotide 6-mer sequences
cagaga

SEQ ID NO: 800 - DNA, 6 bp, Artificial Sequence
  oligonucleotide 6-mer sequences
cgcaga

SEQ ID NO: 801 - DNA, 6 bp, Artificial Sequence
  oligonucleotide 6-mer sequences
tacaga

SEQ ID NO: 802 - DNA, 6 bp, Artificial Sequence
  oligonucleotide 6-mer sequences
aacaga

SEQ ID NO: 803 - DNA, 6 bp, Artificial Sequence
  oligonucleotide 6-mer sequences
gtaaga

SEQ ID NO: 804 - DNA, 6 bp, Artificial Sequence
  oligonucleotide 6-mer sequences
ataaga

SEQ ID NO: 805 - DNA, 6 bp, Artificial Sequence
  oligonucleotide 6-mer sequences
tgaaga

SEQ ID NO: 806 - DNA, 6 bp, Artificial Sequence
  oligonucleotide 6-mer sequences
cgaaga

SEQ ID NO: 807 - DNA, 6 bp, Artificial Sequence
  oligonucleotide 6-mer sequences
acaaga

SEQ ID NO: 808 - DNA, 6 bp, Artificial Sequence
  oligonucleotide 6-mer sequences
taaaga

SEQ ID NO: 809 - DNA, 6 bp, Artificial Sequence
  oligonucleotide 6-mer sequences
gattca

SEQ ID NO: 810 - DNA, 6 bp, Artificial Sequence
  oligonucleotide 6-mer sequences
ccctca

SEQ ID NO: 811 - DNA, 6 bp, Artificial Sequence
  oligonucleotide 6-mer sequences
tactca

SEQ ID NO: 812 - DNA, 6 bp, Artificial Sequence
  oligonucleotide 6-mer sequences
aactca

SEQ ID NO: 813 - DNA, 6 bp, Artificial Sequence
  oligonucleotide 6-mer sequences
gtatca

SEQ ID NO: 814 - DNA, 6 bp, Artificial Sequence
  oligonucleotide 6-mer sequences
tgatca

SEQ ID NO: 815 - DNA, 6 bp, Artificial Sequence
  oligonucleotide 6-mer sequences
caatca

SEQ ID NO: 816 - DNA, 6 bp, Artificial Sequence
  oligonucleotide 6-mer sequences
gttgca

SEQ ID NO: 817 - DNA, 6 bp, Artificial Sequence
  oligonucleotide 6-mer sequences
tgtgca

SEQ ID NO: 818 - DNA, 6 bp, Artificial Sequence
  oligonucleotide 6-mer sequences
tatgca

SEQ ID NO: 819 - DNA, 6 bp, Artificial Sequence
  oligonucleotide 6-mer sequences
ccggca

SEQ ID NO: 820 - DNA, 6 bp, Artificial Sequence
  oligonucleotide 6-mer sequences
acggca

SEQ ID NO: 821 - DNA, 6 bp, Artificial Sequence
  oligonucleotide 6-mer sequences
gtcgca

SEQ ID NO: 822 - DNA, 6 bp, Artificial Sequence
  oligonucleotide 6-mer sequences
tgcgca

SEQ ID NO: 823 - DNA, 6 bp, Artificial Sequence
  oligonucleotide 6-mer sequences
agcgca

SEQ ID NO: 824 - DNA, 6 bp, Artificial Sequence
  oligonucleotide 6-mer sequences
tacgca

SEQ ID NO: 825 - DNA, 6 bp, Artificial Sequence
  oligonucleotide 6-mer sequences
gtagca

SEQ ID NO: 826 - DNA, 6 bp, Artificial Sequence
  oligonucleotide 6-mer sequences
atagca

SEQ ID NO: 827 - DNA, 6 bp, Artificial Sequence
  oligonucleotide 6-mer sequences
ggagca

SEQ ID NO: 828 - DNA, 6 bp, Artificial Sequence
  oligonucleotide 6-mer sequences
taagca

SEQ ID NO: 829 - DNA, 6 bp, Artificial Sequence
  oligonucleotide 6-mer sequences
aaagca

SEQ ID NO: 830 - DNA, 6 bp, Artificial Sequence
  oligonucleotide 6-mer sequences
gtccca

SEQ ID NO: 831 - DNA, 6 bp, Artificial Sequence
  oligonucleotide 6-mer sequences
agccca

SEQ ID NO: 832 - DNA, 6 bp, Artificial Sequence
  oligonucleotide 6-mer sequences
gtacca

SEQ ID NO: 833 - DNA, 6 bp, Artificial Sequence
  oligonucleotide 6-mer sequences
atacca

SEQ ID NO: 834 - DNA, 6 bp, Artificial Sequence
  oligonucleotide 6-mer sequences
tgacca

SEQ ID NO: 835 - DNA, 6 bp, Artificial Sequence
  oligonucleotide 6-mer sequences
cttaca

SEQ ID NO: 836 - DNA, 6 bp, Artificial Sequence
  oligonucleotide 6-mer sequences
ggtaca

SEQ ID NO: 837 - DNA, 6 bp, Artificial Sequence
  oligonucleotide 6-mer sequences
cgtaca

SEQ ID NO: 838 - DNA, 6 bp, Artificial Sequence
  oligonucleotide 6-mer sequences
agtaca

SEQ ID NO: 839 - DNA, 6 bp, Artificial Sequence
  oligonucleotide 6-mer sequences
gctaca

SEQ ID NO: 840 - DNA, 6 bp, Artificial Sequence
  oligonucleotide 6-mer sequences
actaca

SEQ ID NO: 841 - DNA, 6 bp, Artificial Sequence
  oligonucleotide 6-mer sequences
tataca

SEQ ID NO: 842 - DNA, 6 bp, Artificial Sequence
  oligonucleotide 6-mer sequences
gataca

SEQ ID NO: 843 - DNA, 6 bp, Artificial Sequence
  oligonucleotide 6-mer sequences
aataca

SEQ ID NO: 844 - DNA, 6 bp, Artificial Sequence
  oligonucleotide 6-mer sequences
gtgaca

SEQ ID NO: 845 - DNA, 6 bp, Artificial Sequence
  oligonucleotide 6-mer sequences
atgaca

SEQ ID NO: 846 - DNA, 6 bp, Artificial Sequence
  oligonucleotide 6-mer sequences
tcgaca

SEQ ID NO: 847 - DNA, 6 bp, Artificial Sequence
  oligonucleotide 6-mer sequences
gcgaca

SEQ ID NO: 848 - DNA, 6 bp, Artificial Sequence
  oligonucleotide 6-mer sequences
acgaca

SEQ ID NO: 849 - DNA, 6 bp, Artificial Sequence
  oligonucleotide 6-mer sequences
aagaca

SEQ ID NO: 850 - DNA, 6 bp, Artificial Sequence
  oligonucleotide 6-mer sequences
tgcaca

SEQ ID NO: 851 - DNA, 6 bp, Artificial Sequence
  oligonucleotide 6-mer sequences
gacaca

SEQ ID NO: 852 - DNA, 6 bp, Artificial Sequence
  oligonucleotide 6-mer sequences
ttaaca

SEQ ID NO: 853 - DNA, 6 bp, Artificial Sequence
  oligonucleotide 6-mer sequences
cgaaca

SEQ ID NO: 854 - DNA, 6 bp, Artificial Sequence
  oligonucleotide 6-mer sequences
agaaca

SEQ ID NO: 855 - DNA, 6 bp, Artificial Sequence
  oligonucleotide 6-mer sequences
caaaca

SEQ ID NO: 856 - DNA, 6 bp, Artificial Sequence
  oligonucleotide 6-mer sequences
gtttaa

SEQ ID NO: 857 - DNA, 6 bp, Artificial Sequence
  oligonucleotide 6-mer sequences
tgttaa

SEQ ID NO: 858 - DNA, 6 bp, Artificial Sequence
  oligonucleotide 6-mer sequences
ggttaa

SEQ ID NO: 859 - DNA, 6 bp, Artificial Sequence
  oligonucleotide 6-mer sequences
agttaa

SEQ ID NO: 860 - DNA, 6 bp, Artificial Sequence
  oligonucleotide 6-mer sequences
tattaa

SEQ ID NO: 861 - DNA, 6 bp, Artificial Sequence
  oligonucleotide 6-mer sequences
cattaa

SEQ ID NO: 862 - DNA, 6 bp, Artificial Sequence
  oligonucleotide 6-mer sequences
ttgtaa

SEQ ID NO: 863 - DNA, 6 bp, Artificial Sequence
  oligonucleotide 6-mer sequences
gtgtaa

SEQ ID NO: 864 - DNA, 6 bp, Artificial Sequence
  oligonucleotide 6-mer sequences
atgtaa

SEQ ID NO: 865 - DNA, 6 bp, Artificial Sequence
  oligonucleotide 6-mer sequences
cggtaa

SEQ ID NO: 866 - DNA, 6 bp, Artificial Sequence
  oligonucleotide 6-mer sequences
aggtaa

SEQ ID NO: 867 - DNA, 6 bp, Artificial Sequence
  oligonucleotide 6-mer sequences
tcgtaa

SEQ ID NO: 868 - DNA, 6 bp, Artificial Sequence
  oligonucleotide 6-mer sequences
ccgtaa

SEQ ID NO: 869 - DNA, 6 bp, Artificial Sequence
  oligonucleotide 6-mer sequences
acgtaa

SEQ ID NO: 870 - DNA, 6 bp, Artificial Sequence
  oligonucleotide 6-mer sequences
tagtaa

SEQ ID NO: 871 - DNA, 6 bp, Artificial Sequence
  oligonucleotide 6-mer sequences
gagtaa

SEQ ID NO: 872 - DNA, 6 bp, Artificial Sequence
  oligonucleotide 6-mer sequences
tgctaa

SEQ ID NO: 873 - DNA, 6 bp, Artificial Sequence
  oligonucleotide 6-mer sequences
ggctaa

SEQ ID NO: 874 - DNA, 6 bp, Artificial Sequence
  oligonucleotide 6-mer sequences
cgctaa

SEQ ID NO: 875 - DNA, 6 bp, Artificial Sequence
  oligonucleotide 6-mer sequences
gcctaa

SEQ ID NO: 876 - DNA, 6 bp, Artificial Sequence
  oligonucleotide 6-mer sequences
ccctaa

SEQ ID NO: 877 - DNA, 6 bp, Artificial Sequence
  oligonucleotide 6-mer sequences
acctaa

SEQ ID NO: 878 - DNA, 6 bp, Artificial Sequence
  oligonucleotide 6-mer sequences
tactaa

SEQ ID NO: 879 - DNA, 6 bp, Artificial Sequence
  oligonucleotide 6-mer sequences
ttataa

SEQ ID NO: 880 - DNA, 6 bp, Artificial Sequence
  oligonucleotide 6-mer sequences
gtataa

SEQ ID NO: 881 - DNA, 6 bp, Artificial Sequence
  oligonucleotide 6-mer sequences
ctataa

SEQ ID NO: 882 - DNA, 6 bp, Artificial Sequence
  oligonucleotide 6-mer sequences
atataa

SEQ ID NO: 883 - DNA, 6 bp, Artificial Sequence
  oligonucleotide 6-mer sequences
cgataa

SEQ ID NO: 884 - DNA, 6 bp, Artificial Sequence
  oligonucleotide 6-mer sequences
agataa

SEQ ID NO: 885 - DNA, 6 bp, Artificial Sequence
  oligonucleotide 6-mer sequences
gcataa

SEQ ID NO: 886 - DNA, 6 bp, Artificial Sequence
  oligonucleotide 6-mer sequences
acataa

SEQ ID NO: 887 - DNA, 6 bp, Artificial Sequence
  oligonucleotide 6-mer sequences
taataa

SEQ ID NO: 888 - DNA, 6 bp, Artificial Sequence
  oligonucleotide 6-mer sequences
caataa

SEQ ID NO: 889 - DNA, 6 bp, Artificial Sequence
  oligonucleotide 6-mer sequences
cgtgaa

SEQ ID NO: 890 - DNA, 6 bp, Artificial Sequence
  oligonucleotide 6-mer sequences
gatgaa

SEQ ID NO: 891 - DNA, 6 bp, Artificial Sequence
  oligonucleotide 6-mer sequences
catgaa

SEQ ID NO: 892 - DNA, 6 bp, Artificial Sequence
  oligonucleotide 6-mer sequences
ttggaa

SEQ ID NO: 893 - DNA, 6 bp, Artificial Sequence
  oligonucleotide 6-mer sequences
tgcgaa

SEQ ID NO: 894 - DNA, 6 bp, Artificial Sequence
  oligonucleotide 6-mer sequences
agcgaa

SEQ ID NO: 895 - DNA, 6 bp, Artificial Sequence
  oligonucleotide 6-mer sequences
accgaa

SEQ ID NO: 896 - DNA, 6 bp, Artificial Sequence
  oligonucleotide 6-mer sequences
aacgaa

SEQ ID NO: 897 - DNA, 6 bp, Artificial Sequence
  oligonucleotide 6-mer sequences
ttagaa

SEQ ID NO: 898 - DNA, 6 bp, Artificial Sequence
  oligonucleotide 6-mer sequences
gtagaa

SEQ ID NO: 899 - DNA, 6 bp, Artificial Sequence
  oligonucleotide 6-mer sequences
ggagaa

SEQ ID NO: 900 - DNA, 6 bp, Artificial Sequence
  oligonucleotide 6-mer sequences
ggtcaa

SEQ ID NO: 901 - DNA, 6 bp, Artificial Sequence
  oligonucleotide 6-mer sequences
cctcaa

SEQ ID NO: 902 - DNA, 6 bp, Artificial Sequence
  oligonucleotide 6-mer sequences
catcaa

SEQ ID NO: 903 - DNA, 6 bp, Artificial Sequence
  oligonucleotide 6-mer sequences
ctgcaa

SEQ ID NO: 904 - DNA, 6 bp, Artificial Sequence
  oligonucleotide 6-mer sequences
atgcaa

SEQ ID NO: 905 - DNA, 6 bp, Artificial Sequence
  oligonucleotide 6-mer sequences
cggcaa

SEQ ID NO: 906 - DNA, 6 bp, Artificial Sequence
  oligonucleotide 6-mer sequences
tcgcaa

SEQ ID NO: 907 - DNA, 6 bp, Artificial Sequence
  oligonucleotide 6-mer sequences
ccgcaa

SEQ ID NO: 908 - DNA, 6 bp, Artificial Sequence
  oligonucleotide 6-mer sequences
tagcaa

SEQ ID NO: 909 - DNA, 6 bp, Artificial Sequence
  oligonucleotide 6-mer sequences
gagcaa

SEQ ID NO: 910 - DNA, 6 bp, Artificial Sequence
  oligonucleotide 6-mer sequences
ttacaa

SEQ ID NO: 911 - DNA, 6 bp, Artificial Sequence
  oligonucleotide 6-mer sequences
ctacaa

SEQ ID NO: 912 - DNA, 6 bp, Artificial Sequence
  oligonucleotide 6-mer sequences
atacaa

SEQ ID NO: 913 - DNA, 6 bp, Artificial Sequence
  oligonucleotide 6-mer sequences
tgacaa

SEQ ID NO: 914 - DNA, 6 bp, Artificial Sequence
  oligonucleotide 6-mer sequences
ggacaa

SEQ ID NO: 915 - DNA, 6 bp, Artificial Sequence
  oligonucleotide 6-mer sequences
cgacaa

SEQ ID NO: 916 - DNA, 6 bp, Artificial Sequence
  oligonucleotide 6-mer sequences
gcacaa

SEQ ID NO: 917 - DNA, 6 bp, Artificial Sequence
  oligonucleotide 6-mer sequences
acacaa

SEQ ID NO: 918 - DNA, 6 bp, Artificial Sequence
  oligonucleotide 6-mer sequences
taacaa

SEQ ID NO: 919 - DNA, 6 bp, Artificial Sequence
  oligonucleotide 6-mer sequences
aaacaa

SEQ ID NO: 920 - DNA, 6 bp, Artificial Sequence
  oligonucleotide 6-mer sequences
tgtaaa

SEQ ID NO: 921 - DNA, 6 bp, Artificial Sequence
  oligonucleotide 6-mer sequences
cgtaaa

SEQ ID NO: 922 - DNA, 6 bp, Artificial Sequence
  oligonucleotide 6-mer sequences
gctaaa

SEQ ID NO: 923 - DNA, 6 bp, Artificial Sequence
  oligonucleotide 6-mer sequences
cctaaa

SEQ ID NO: 924 - DNA, 6 bp, Artificial Sequence
  oligonucleotide 6-mer sequences
tataaa

SEQ ID NO: 925 - DNA, 6 bp, Artificial Sequence
  oligonucleotide 6-mer sequences
cataaa

SEQ ID NO: 926 - DNA, 6 bp, Artificial Sequence
  oligonucleotide 6-mer sequences
gcgaaa

SEQ ID NO: 927 - DNA, 6 bp, Artificial Sequence
  oligonucleotide 6-mer sequences
tgcaaa

SEQ ID NO: 928 - DNA, 6 bp, Artificial Sequence
  oligonucleotide 6-mer sequences
cgcaaa

SEQ ID NO: 929 - DNA, 6 bp, Artificial Sequence
  oligonucleotide 6-mer sequences
tccaaa

SEQ ID NO: 930 - DNA, 6 bp, Artificial Sequence
  oligonucleotide 6-mer sequences
ctaaaa

SEQ ID NO: 931 - DNA, 6 bp, Artificial Sequence
  oligonucleotide 6-mer sequences
ggaaaa

SEQ ID NO: 932 - DNA, 6 bp, Artificial Sequence
  oligonucleotide 6-mer sequences
gcaaaa

SEQ ID NO: 933 - DNA, 6 bp, Artificial Sequence
  oligonucleotide 6-mer sequences
taaaaa

SEQ ID NO: 934 - DNA, 26 bp, Artificial Sequence
  T7 bacteriophage promoter
aattaatacgactcactatagggaga

SEQ ID NO: 935 - DNA, 23 bp, Artificial Sequence
  misc_feature 22: n = A,T,C or G
atttaggtgacactatagaagng

SEQ ID NO: 936 - DNA, 23 bp, Artificial Sequence
  T3 bacteriophage promoter
aattaaccctcactaaagggaga

SEQ ID NO: 937 - DNA, 15 bp, Artificial Sequence
  Oligonucleotide
gcacgcgcacaagct

SEQ ID NO: 938 - DNA, 16 bp, Artificial Sequence
  Oligonucleotide
gggtcaccagcaggca

SEQ ID NO: 939 - DNA, 20 bp, Artificial Sequence
  Oligonucleotide
acttcaagctcctaagccac

SEQ ID NO: 940 - DNA, 18 bp, Artificial Sequence
  Synthetic primer
acggtggccatggaagtc

SEQ ID NO: 941 - DNA, 21 bp, Artificial Sequence
  Synthetic primer
tcggcaggtgagttgttacac

SEQ ID NO: 942 - DNA, 17 bp, Artificial Sequence
  Synthetic primer
actccttagcggattcc

SEQ ID NO: 943 - DNA, 15 bp, Artificial Sequence
  Synthetic primer
gcacgcgcacaagct

SEQ ID NO: 944 - DNA, 16 bp, Artificial Sequence
  Synthetic primer
gggtcaccagcaggca

SEQ ID NO: 945 - DNA, 20 bp, Artificial Sequence
  Synthetic primer
acttcaagctcctaagccac

SEQ ID NO: 946 - DNA, 25 bp, Artificial Sequence
  Synthetic primer
cgcaattaatacgactcactatagg

SEQ ID NO: 947 - DNA, 31 bp, Artificial Sequence
  Synthetic primer
tgcattgagagggtgtaatttgnnnnnnnnn

SEQ ID NO: 948 - DNA, 22 bp, Artificial Sequence
  Synthetic primer
tgcattgagagggtgtaatttg

SEQ ID NO: 949 - DNA, 31 bp, Artificial Sequence
  Synthetic primer
gtatcttgggcctgttgacttcnnnnnnnnn

SEQ ID NO: 950 - DNA, 22 bp, Artificial Sequence
  Synthetic primer
gtatcttgggcctgttgacttc

SEQ ID NO: 951 - DNA, 31 bp, Artificial Sequence
  Synthetic primer
attgttccttcagctgttccatnnnnnnnnn

SEQ ID NO: 952 - DNA, 22 bp, Artificial Sequence
  Synthetic primer
attgttccttcagctgttccat

SEQ ID NO: 953 - DNA, 31 bp, Artificial Sequence
  Synthetic primer
cctccacactcctaaggtcatcnnnnnnnnn

SEQ ID NO: 954 - DNA, 22 bp, Artificial Sequence
  Synthetic primer
cctccacactcctaaggtcatc

SEQ ID NO: 955 - DNA, 22 bp, Artificial Sequence
  Synthetic primer
gcgcaattaatacgactcacta

SEQ ID NO: 956 - DNA, 20 bp, Artificial Sequence
  Synthetic primer
ccgaactacccacttgcatt

SEQ ID NO: 957 - DNA, 29 bp, Artificial Sequence
  Synthetic primer
ccactccatttgttcgtgtgnnnnnnnnn

SEQ ID NO: 958 - DNA, 20 bp, Artificial Sequence
  Synthetic primer
ccgaactacccacttgcatt

SEQ ID NO: 959 - DNA, 20 bp, Artificial Sequence
  Synthetic primer
ccactccatttgttcgtgtg